SEQUENCE LISTING (34 sequences)

SEQ ID NO: 1 - protein, 184 aa, synthetic construct
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 2 - protein, 207 aa, synthetic construct
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 3 - protein, 104 aa, synthetic construct
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 4 - protein, 7 aa, synthetic construct
SGSYFWG

SEQ ID NO: 5 - protein, 16 aa, synthetic construct
SIYYSGITYYNPSLKS

SEQ ID NO: 6 - protein, 11 aa, synthetic construct
HDGAVAGLFDY

SEQ ID NO: 7 - protein, 11 aa, synthetic construct
GGNNIGSKSVH

SEQ ID NO: 8 - protein, 7 aa, synthetic construct
DDSDRPS

SEQ ID NO: 9 - protein, 11 aa, synthetic construct
QVWDSSSDHVV

SEQ ID NO: 10 - protein, 121 aa, synthetic construct
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSS

SEQ ID NO: 11 - protein, 108 aa, synthetic construct
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVL

SEQ ID NO: 12 - protein, 448 aa, synthetic construct
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 214 aa, synthetic construct
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 14 - protein, 5 aa, synthetic construct
TYAMN

SEQ ID NO: 15 - protein, 19 aa, synthetic construct
RIRSKYNNYATYYAASVKG

SEQ ID NO: 16 - protein, 14 aa, synthetic construct
HGNFGNSYVSWFAY

SEQ ID NO: 17 - protein, 14 aa, synthetic construct
RSSTGAVTTSNYAN

SEQ ID NO: 18 - protein, 7 aa, synthetic construct
GTNKRAP

SEQ ID NO: 19 - protein, 9 aa, synthetic construct
ALWYSNLWV

SEQ ID NO: 20 - protein, 125 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSS

SEQ ID NO: 21 - protein, 112 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQP

SEQ ID NO: 22 - protein, 452 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 23 - protein, 215 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 24 - protein, 5 aa, synthetic construct
GYTMN

SEQ ID NO: 25 - protein, 17 aa, synthetic construct
LINPYNSDTNYAQKLQG

SEQ ID NO: 26 - protein, 9 aa, synthetic construct
VALRVALDY

SEQ ID NO: 27 - protein, 11 aa, synthetic construct
KASQNVATHVG

SEQ ID NO: 28 - protein, 7 aa, synthetic construct
SASYRYS

SEQ ID NO: 29 - protein, 9 aa, synthetic construct
QQYNRYPYT

SEQ ID NO: 30 - protein, 118 aa, synthetic construct
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSS

SEQ ID NO: 31 - protein, 107 aa, synthetic construct
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIK

SEQ ID NO: 32 - protein, 445 aa, synthetic construct
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 214 aa, synthetic construct
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2024/0415960 A1
(43) Pub. Date: Dec. 19, 2024

(54) METHODS FOR TREATING MULTIPLE MYELOMA

(71) Applicant: Janssen Biotech, Inc., Horsham, PA (US)

(72) Inventors: Rachel KOBOS, Bridgewater, NJ (US); Arnob BANERJEE, Baltimore, MD (US); Jenna GOLDBERG, Livingston, NJ (US); Christoph HEUCK, Princeton, NJ (US)

(21) Appl. No.: 18/639,233

(22) Filed: Apr. 18, 2024

Related U.S. Application Data
(60) Provisional application No. 63/621,871, filed on Jan. 17, 2024.
(60) Provisional application No. 63/588,480, filed on Oct. 6, 2023.
(60) Provisional application No. 63/505,594, filed on Jun. 1, 2023.
(60) Provisional application No. 63/504,150, filed on May 24, 2023.
(60) Provisional application No. 63/497,119, filed on Apr. 19, 2023.

(51) Int. Cl. A61K 39/395; A61K 39/00; A61P 35/00; C07K 16/28

(52) U.S. Cl. CPC A61K 39/39558; A61K 39/39541; A61P 35/00; C07K 16/2809; C07K 16/2866; C07K 16/2878; A61K 2039/507; A61K 2039/545; C07K 2317/24; C07K 2317/31

(57) ABSTRACT

Embodiments of the present invention relate to methods of treating multiple myeloma in a subject in need thereof, comprising administering therapeutically effective amounts of a BCMA×CD3 bispecific antibody and a GPRC5D×CD3 bispecific antibody to the subject.

### CROSS REFERENCE TO RELATED APPLICATIONS

This application claims the benefit of U.S. Provisional Application Ser. No. 63/497,119 filed on Apr. 19, 2023; 63/504,150 filed on May 24, 2023; 63/505,594, filed on Jun. 1, 2023; 63/588,480 filed on Oct. 6, 2023; and 63/621,871 filed on Jan. 17, 2024. The entire content of the aforementioned applications are incorporated herein by reference in their entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “258199061301(JBI6800USNP1)_Sequence_Listing.xml”, was created on Apr. 18, 2024 and is 33,898 bytes in size.

### FIELD OF THE INVENTION

Methods of treating multiple myeloma are disclosed.

### BACKGROUND OF THE INVENTION

Multiple myeloma (MM) is a cancer of the plasma cells. Mechanistically, multiple myeloma is characterized by production of monoclonal proteins (M-proteins) comprised of pathological immunoglobulins or fragments of such, which have lost their function. The proliferation of multiple myeloma cells leads to subsequent displacement from the normal bone marrow niche, w % bile overproduction of M-proteins causes characteristic osteolytic lesions, increased susceptibility to infections, hypercalcemia, renal insufficiency or failure, and neurological complications.

Extramedullary disease (EMD) is an aggressive form of multiple myeloma, characterized by the ability of malignant plasma cells to thrive and grow independent of the bone marrow microenvironment, resulting in organ infiltration and soft tissue plasmacytoma not contiguous with bone. In patients with newly diagnosed multiple myeloma, the reported incidence ranges from 0.5% to 4.8%, while in relapsed/refractory multiple myeloma the reported incidence is 3.4% to 14%.

Treatment options for multiple myeloma have improved over time and vary depending on the aggressiveness of the disease, underlying prognostic factors, physical condition of the patient, and existing comorbidities. Therapeutic options include proteasome inhibitors (PIs), immunomodulatory drugs (IMiDs), alkylating agents, monoclonal antibodies (mAbs), antibody drug conjugate, histone deacetylase inhibitor, nuclear protein export inhibitor, chimeric antigen receptor (CAR) T cell therapy and stem cell transplantation.

Despite these therapeutic achievements, the disease recurs and is associated with additional risk factors (e.g., comorbidities or increasing age), thus warranting the need for novel therapeutic approaches, such as new dosage and treatment regimens. In particular in the elderly population, for which stem cell transplantation is often not a viable option, and in patients with refractory disease who exhausted several therapies, multiple myeloma remains an incurable malignancy and an unmet medical need with significant morbidity and mortality. In particular, there remains a need for therapeutic regimens that achieve rapid, deep and durable clinical responses, while providing manageable safety profiles.

### SUMMARY OF THE INVENTION

Embodiments of the present invention provide combination regimens for the treatment of multiple myeloma comprising a BCMA×CD3 bispecific antibody and a GPRC5D×CD3 bispecific antibody.

An embodiment of the present invention provides a method of treating multiple myeloma in a subject in need thereof, comprising administering to the subject a combination dosing regimen comprising a therapeutically effective amount of a BCMA×CD3 bispecific antibody and a therapeutically effective amount of a GPRC5D×CD3 bispecific antibody. In certain embodiments, the subject has relapsed or refractory multiple myeloma and received at least three prior lines of therapy, including a proteasome inhibitor, an immunomodulatory agent and an anti-CD38 monoclonal antibody.

In certain embodiments, the BCMA×CD3 bispecific antibody comprises a BCMA binding domain comprising the HCDR1 of SEQ ID NO: 4, the HCDR2 of SEQ ID NO: 5, the HCDR3 of SEQ ID NO: 6, the LCDR1 of SEQ ID NO: 7, the LCDR2 of SEQ ID NO: 8 and the LCDR3 of SEQ ID NO: 9, and a CD3 binding domain comprising the HCDR1 of SEQ ID NO: 14, the HCDR2 of SEQ ID NO: 15, the HCDR3 of SEQ ID NO: 16, the LCDR1 of SEQ ID NO: 17, the LCDR2 of SEQ ID NO: 18 and the LCDR3 of SEQ ID NO: 19.

In certain embodiments, the BCMA binding domain comprises a heavy chain variable region (VH) having the amino acid sequence of SEQ ID NO: 10 and a light chain variable region (VL) having the amino acid sequence of SEQ ID NO: 11, and the CD3 binding domain comprises a heavy chain variable region (VH) having the amino acid sequence of SEQ ID NO: 20 and a light chain variable region (VL) having the amino acid sequence of SEQ ID NO: 21.

In certain embodiments, the BCMA×CD3 bispecific antibody is an IgG1, an IgG2, an IgG3 or an IgG4 isotype.

In certain embodiments, the BCMA×CD3 bispecific antibody is an IgG4 isotype.

In certain embodiments, the BCMA×CD3 bispecific antibody comprises one or more substitutions in its Fc region.

In certain embodiments, the BCMA×CD3 bispecific antibody is an IgG4 isotype and comprises S228P, F234A and L235A substitutions in its Fc region.

In certain embodiments, the BCMA×CD3 bispecific antibody is an IgG4 isotype and comprises S228P, F234A, L235A F405L and R409K substitutions in its Fc region.

In certain embodiments, the Fc region of the BCMA-specific IgG4 antibody from which the BCMA-binding arm is derived comprises S228P. L234A and L235A substitutions in its Fc region.

In certain embodiments, the Fc region of the CD3-specific IgG4 antibody from which the CD3-binding arm is derived comprises S228P, L234A, L235A, F405L, and R409K substitutions in its Fc region. In certain embodiments, the BCMA×CD3 bispecific antibody comprises a first heavy chain (HC1) having the amino acid sequence of SEQ ID NO: 12, a first light chain (LC1) having the amino acid sequence of SEQ ID NO: 13, a second heavy chain (HC2) having the amino acid sequence of SEQ ID NO: 22 and a second light chain (LC2) having the amino acid sequence of SEQ ID NO: 23.

In certain embodiments, the BCMA×CD3 bispecific antibody comprises a first heavy chain (HC1) having at least 90% identity to the amino acid sequence of SEQ ID NO: 12, a first light chain (LC1) having at least 90% identity to the amino acid sequence of SEQ ID NO: 13, a second heavy chain (HC2) having at least 90% identity to the amino acid sequence of SEQ ID NO: 22 and a second light chain (LC2) having at least 90% identity to the amino acid sequence of SEQ ID NO: 23.

In certain embodiments, the BCMA×CD3 bispecific antibody comprises a first heavy chain (HC1) having at least 95% identity to the amino acid sequence of SEQ ID NO: 12, a first light chain (LC1) having at least 95% identity to the amino acid sequence of SEQ ID NO: 13, a second heavy chain (HC2) having at least 95% identity to the amino acid sequence of SEQ ID NO: 22 and a second light chain (LC2) having at least 95% identity to the amino acid sequence of SEQ ID NO: 23.

In certain embodiments, the BCMA×CD3 bispecific antibody comprises a first heavy chain (HC1) having at least 98% identity to the amino acid sequence of SEQ ID NO: 12, a first light chain (LC1) having at least 98% identity to the amino acid sequence of SEQ ID NO: 13, a second heavy chain (HC2) having at least 98% identity to the amino acid sequence of SEQ ID NO: 22 and a second light chain (LC2) having at least 98% identity to the amino acid sequence of SEQ ID NO: 23.

In certain embodiments, the BCMA×CD3 bispecific antibody is teclistamab.

In certain embodiments, the GPRC5D×CD3 bispecific antibody comprises a GPRC5D binding domain comprising the HCDR1 of SEQ ID NO: 24, the HCDR2 of SEQ ID NO: 25, the HCDR3 of SEQ ID NO: 26, the LCDR1 of SEQ ID NO: 27, the LCDR2 of SEQ ID NO: 28 and the LCDR3 of SEQ ID NO: 29, and a CD3 binding domain comprising the HCDR1 of SEQ ID NO: 14, the HCDR2 of SEQ ID NO: 15, the HCDR3 of SEQ ID NO: 16, the LCDR1 of SEQ ID NO: 17, the LCDR2 of SEQ ID NO: 18 and the LCDR3 of SEQ ID NO: 19.

In certain embodiments, the GPRC5D binding domain comprises a heavy chain variable region (VH) having the amino acid sequence of SEQ ID NO: 30 and a light chain variable region (VL) having the amino acid sequence of SEQ ID NO: 31, and the CD3 binding domain comprises a heavy chain variable region (VH) having the amino acid sequence of SEQ ID NO: 20 and a light chain variable region (VL) having the amino acid sequence of SEQ ID NO: 21.

In certain embodiments, the GPRC5D×CD3 bispecific antibody is an IgG1, an IgG2, an IgG3 or an IgG4 isotype.

In certain embodiments, the GPRC5D×CD3 bispecific antibody is an IgG4 isotype.

In certain embodiments, the GPRC5D×CD3 bispecific antibody comprises one or more substitutions in its Fc region.

In certain embodiments, the GPRC5D×CD3 bispecific antibody is an IgG4 isotype and comprises S228P. F234A and L235A substitutions in its Fc region.

In certain embodiments, the GPRC5D×CD3 bispecific antibody is an IgG4 isotype and comprises S228P, F234A, L235A F405L and R409K substitutions in its Fc region.

In certain embodiments, the Fc region of the GPRC5D-specific IgG4 antibody from which the GPRC5D-binding arm is derived comprises S228P, L234A and L235A substitutions in its Fc region.

In certain embodiments, the Fc region of the CD3-specific IgG4 antibody from which the CD3-binding arm is derived comprises S228P, L234A, L235A, F405L, and R409K substitutions in its Fc region.

In certain embodiments, the GPRC5D×CD3 bispecific antibody comprises a first heavy chain (HC1) having the amino acid sequence of SEQ ID NO: 32, a first light chain (LC1) having the amino acid sequence of SEQ ID NO: 33, a second heavy chain (HC2) having the amino acid sequence of SEQ ID NO: 22 and a second light chain (LC2) having the amino acid sequence of SEQ ID NO: 23.

In certain embodiments, the GPRC5D×CD3 bispecific antibody comprises a first heavy chain (HC1) having at least 90% identity to the amino acid sequence of SEQ ID NO: 32, a first light chain (LC1) having at least 90% identity to the amino acid sequence of SEQ ID NO: 33, a second heavy chain (HC2) having at least 90% identity to the amino acid sequence of SEQ ID NO: 22 and a second light chain (LC2) having at least 90% identity to the amino acid sequence of SEQ ID NO: 23.

In certain embodiments, the GPRC5D×CD3 bispecific antibody comprises a first heavy chain (HC1) having at least 95% identity to the amino acid sequence of SEQ ID NO: 32, a first light chain (LC1) having at least 95% identity to the amino acid sequence of SEQ ID NO: 33, a second heavy chain (HC2) having at least 95% identity to the amino acid sequence of SEQ ID NO: 22 and a second light chain (LC2) having at least 95% identity to the amino acid sequence of SEQ ID NO: 23.

In certain embodiments, the GPRC5D×CD3 bispecific antibody comprises a first heavy chain (HC1) having at least 98% identity to the amino acid sequence of SEQ ID NO: 32, a first light chain (LC1) having at least 98% identity to the amino acid sequence of SEQ ID NO: 33, a second heavy chain (HC2) having at least 98% identity to the amino acid sequence of SEQ ID NO: 22 and a second light chain (LC2) having at least 98% identity to the amino acid sequence of SEQ ID NO: 23.

In certain embodiments, the GPRC5D×CD3 bispecific antibody is talquetamab.

In certain embodiments, the subject has relapsed or refractory multiple myeloma.

In certain embodiments, the subject has had been treated with from 1 to 11 prior lines of therapy, or from 1 to 10 prior lines of therapy.

In certain embodiments, the subject has previously received an autologous stem cell transplant (ASCT).

In certain embodiments, the subject has received at least three prior lines of therapy.

In certain embodiments, the subject has received at least four prior lines of therapy.

In certain embodiments, the subject has received at least five prior lines of therapy (penta-drug exposed).

In certain embodiments, the subject has received at least three prior lines of therapy, including a proteasome inhibitor, an immunomodulatory agent and an anti-CD38 monoclonal antibody.

In certain embodiments, the subject has received at least four prior lines of therapy, including a proteasome inhibitor, an immunomodulatory agent and an anti-CD38 monoclonal antibody.

In certain embodiments, the subject has extramedullary disease (EMD).

In certain embodiments, the method comprises subcutaneously administering to the subject one or more step-up doses of the BCMA×CD3 bispecific antibody prior to administering a treatment dose of the BCMA×CD3 bispecific antibody.

In certain embodiments, the method comprises subcutaneously administering to the subject a treatment dose of the BCMA×CD3 bispecific antibody weekly (QW).

In certain embodiments, the method comprises subcutaneously administering to the subject a treatment dose of the BCMA×CD3 bispecific antibody every two weeks (Q2W).

In certain embodiments, the method comprises subcutaneously administering the BCMA×CD3 bispecific antibody weekly (QW) at a treatment dose of from about 750 μg/kg to about 3000 μg/kg.

In certain embodiments, the method comprises subcutaneously administering the BCMA×CD3 bispecific antibody weekly (QW) at a treatment dose of about 750 μg/kg.

In certain embodiments, the method comprises subcutaneously administering the BCMA×CD3 bispecific antibody weekly (QW) at a treatment dose of about 1500 μg/kg.

In certain embodiments, the method comprises subcutaneously administering the BCMA×CD3 bispecific antibody weekly (QW) at a treatment dose of about 3000 μg/kg.

In certain embodiments, the method comprises subcutaneously administering the BCMA×CD3 bispecific antibody every two weeks (Q2W) at a treatment dose of from about 750 μg/kg to about 3000 μg/kg.

In certain embodiments, the method comprises subcutaneously administering the BCMA×CD3 bispecific antibody every two weeks (Q2W) at a treatment dose of 750 μg/kg.

In certain embodiments, the method comprises subcutaneously administering the BCMA×CD3 bispecific antibody every two weeks (Q2W) at a treatment dose of about 1500 μg/kg.

In certain embodiments, the method comprises subcutaneously administering the BCMA×CD3 bispecific antibody every two weeks (Q2W) at a treatment dose of about 3000 μg/kg.

In certain embodiments, the method comprises subcutaneously administering 2 or 3 step-up doses of the BCMA×CD3 bispecific antibody prior to subcutaneously administering the treatment dose.

In certain embodiments, the method comprises subcutaneously administering step-up doses of 60 μg/kg and 300 μg/kg of the BCMA×CD3 bispecific antibody prior to subcutaneously administering a treatment dose.

In certain embodiments, the method comprises subcutaneously administering step-up doses of 60 μg/kg and 300 μg/kg and 1500 μg/kg of the BCMA×CD3 bispecific antibody prior to subcutaneously administering a treatment dose.

In certain embodiments, the method comprises subcutaneously administering step-up doses of the BCMA×CD3 bispecific antibody 2-4 days apart from each other.

In certain embodiments, prior to subcutaneously administering a treatment dose of the BCMA×CD3 bispecific antibody, the method comprises: subcutaneously administering a first step-up dose of 60 μg/kg of the BCMA×CD3 bispecific antibody, then 2-4 days later subcutaneously administering a second step-up dose of 300 μg/kg of the BCMA×CD3 bispecific antibody, then 2-4 days later subcutaneously administering a third step-up dose of 1500 μg/kg of the BCMA×CD3 bispecific antibody.

In certain embodiments, the method comprises subcutaneously administering to the subject one or more step-up doses of the GPRC5D×CD3 bispecific antibody prior to administering a treatment dose of the GPRC5D×CD3 bispecific antibody.

In certain embodiments, the method comprises subcutaneously administering to the subject a treatment dose of the GPRC5D×CD3 bispecific antibody weekly (QW).

In certain embodiments, the method comprises subcutaneously administering to the subject a treatment dose of the GPRC5D×CD3 bispecific antibody every two weeks (Q2W).

In certain embodiments, the method comprises subcutaneously administering the GPRC5D×CD3 bispecific antibody weekly (QW) at a treatment dose of from about 200 μg/kg to about 800 μg/kg.

In certain embodiments, the method comprises subcutaneously administering the GPRC5D×CD3 bispecific antibody weekly (QW) at a treatment dose of about 200 μg/kg.

In certain embodiments, the method comprises subcutaneously administering the GPRC5D×CD3 bispecific antibody weekly (QW) at a treatment dose of about 400 μg/kg.

In certain embodiments, the method comprises subcutaneously administering the GPRC5D×CD3 bispecific antibody weekly (QW) at a treatment dose of about 800 μg/kg.

In certain embodiments, the method comprises subcutaneously administering the GPRC5D×CD3 bispecific antibody every two weeks (Q2W) at a treatment dose of from about 200 μg/kg to about 800 μg/kg.

In certain embodiments, the method comprises subcutaneously administering the GPRC5D×CD3 bispecific antibody every two weeks (Q2W) at a treatment dose of about 200 μg/kg.

In certain embodiments, the method comprises subcutaneously administering the GPRC5D×CD3 bispecific antibody every two weeks (Q2W) at a treatment dose of about 400 μg/kg.

In certain embodiments, the method comprises subcutaneously administering the GPRC5D×CD3 bispecific antibody every two weeks (Q2W) at a treatment dose of about 800 μg/kg.

In certain embodiments, the method comprises subcutaneously administering 2 or 3 step-up doses of the GPRC5D×CD3 bispecific antibody prior to subcutaneously administering the treatment dose.

In certain embodiments, the method comprises subcutaneously administering step-up doses of 10 μg/kg and 60 μg/kg of the GPRC5D×CD3 bispecific antibody prior to subcutaneously administering a treatment dose. In certain embodiments, the method comprises subcutaneously administering step-up doses of 10 μg/kg and 60 μg/kg and 400 μg/kg of the GPRC5D×CD3 bispecific antibody prior to subcutaneously administering a treatment dose.

In certain embodiments, the method comprises subcutaneously administering step-up doses of the GPRC5D×CD3 bispecific antibody 2-4 days apart from each other.

In certain embodiments, prior to subcutaneously administering a treatment dose of the GPRC5D×CD3 bispecific antibody, the method comprises: subcutaneously administering a first step-up dose of 10 μg/kg of the GPRC5D×CD3 bispecific antibody, then 2-4 days later subcutaneously administering a second step-up dose of 60 μg/kg of the GPRC5D×CD3 bispecific antibody, then 2-4 days later subcutaneously administering a third step-up dose of 400 μg/kg of the GPRC5D×CD3 bispecific antibody.

In certain embodiments, the method comprises subcutaneously administering step-up doses of 10 μg/kg and 60 μg/kg and 300 μg/kg of the GPRC5D×CD3 bispecific antibody prior to subcutaneously administering a treatment dose.

In certain embodiments, the method comprises subcutaneously administering step-up doses of the GPRC5D×CD3 bispecific antibody 2-4 days apart from each other.

In certain embodiments, prior to subcutaneously administering a treatment dose of the GPRC5D×CD3 bispecific antibody, the method comprises: subcutaneously administering a first step-up dose of 10 μg/kg of the GPRC5D×CD3 bispecific antibody, then 2-4 days later subcutaneously administering a second step-up dose of 60 μg/kg of the GPRC5D×CD3 bispecific antibody, then 2-4 days later subcutaneously administering a third step-up dose of 300 μg/kg of the GPRC5D×CD3 bispecific antibody.

In certain embodiments, the method comprises subcutaneously administering the BCMA×CD3 bispecific antibody every two weeks (Q2W) at a treatment dose of 3000 μg/kg and subcutaneously administering the GPRC5D×CD3 bispecific antibody every two weeks (Q2W) at a treatment dose of 800 μg/kg.

In certain embodiments, the method comprises subcutaneously administering the BCMA×CD3 bispecific antibody every two weeks (Q2W) at a treatment dose of 1500 μg/kg and subcutaneously administering the GPRC5D×CD3 bispecific antibody every two weeks (Q2W) at a treatment dose of 800 μg/kg.

In certain embodiments, the method comprises subcutaneously administering the BCMA×CD3 bispecific antibody weekly (QW) at a treatment dose of 1500 μg/kg and subcutaneously administering the GPRC5D×CD3 bispecific antibody weekly (QW) at a treatment dose of 800 μg/kg.

In certain embodiments, the method comprises subcutaneously administering the BCMA×CD3 bispecific antibody weekly (QW) at a treatment dose of 1500 μg/kg and subcutaneously administering the GPRC5D×CD3 bispecific antibody weekly (QW) at a treatment dose of 400 μg/kg.

In certain embodiments, the method comprises subcutaneously administering the BCMA×CD3 bispecific antibody weekly (QW) at a treatment dose of 1500 μg/kg and subcutaneously administering the GPRC5D×CD3 bispecific antibody weekly (QW) at a treatment dose of 200 μg/kg.

In certain embodiments, the method comprises subcutaneously administering the BCMA×CD3 bispecific antibody weekly (QW) at a treatment dose of 750 μg/kg and subcutaneously administering the GPRC5D×CD3 bispecific antibody weekly (QW) at a treatment dose of 200 μg/kg.

In certain embodiments, the method comprises subcutaneously administering one or more step-up doses of the BCMA×CD3 bispecific antibody prior to subcutaneously administering a treatment dose; and subcutaneously administering one or more step-up doses of the GPRC5D×CD3 bispecific antibody prior to subcutaneously administering a treatment dose.

In certain embodiments, the method comprises subcutaneously administering 2 or 3 step-up doses of the BCMA×CD3 bispecific antibody prior to subcutaneously administering a treatment dose; and subcutaneously administering 2 or 3 step-up doses of the GPRC5D×CD3 bispecific antibody prior to subcutaneously administering a treatment dose.

In certain embodiments, the method comprises subcutaneously administering step-up doses of 60 μg/kg and 3M) μg/kg of the BCMA×CD3 bispecific antibody prior to subcutaneously administering a treatment dose; and subcutaneously administering step-up doses of 10 μg/kg and 60 μg/kg of the GPRC5D×CD3 bispecific antibody prior to subcutaneously administering a treatment dose.

In certain embodiments, the method comprises subcutaneously administering step-up doses of 60 μg/kg and 300 μg/kg and 1500 μg/kg of the BCMA×CD3 bispecific antibody prior to subcutaneously administering a treatment dose; and subcutaneously administering step-up doses of 10 μg/kg and 60 μg/kg and 300 μg/kg of the GPRC5D×CD3 bispecific antibody prior to subcutaneously administering a treatment dose.

In certain embodiments, the method comprises subcutaneously administering step-up doses of 60 μg/kg and 300 μg/kg and 1500 μg/kg of the BCMA×CD3 bispecific antibody prior to subcutaneously administering a treatment dose; and subcutaneously administering step-up doses of 10 μg/kg and 60 μg/kg and 400 μg/kg of the GPRC5D×CD3 bispecific antibody prior to subcutaneously administering a treatment dose.

In certain embodiments, the step-up doses of the BCMA×CD3 bispecific antibody are administered 2-4 days apart from each other, and the step-up doses of the GPRC5D×CD3 bispecific antibody are administered 2-4 days apart from each other.

In certain embodiments, the method comprises:
subcutaneously administering a first step-up dose of 60 μg/kg of the BCMA×CD3 bispecific antibody, then 2-4 days later subcutaneously administering a second step-up dose of 300 μg/kg of the BCMA×CD3 bispecific antibody, then 2-4 days later subcutaneously administering a third step-up dose of 1500 μg/kg of the BCMA×CD3 bispecific antibody, then 2-4 days later subcutaneously administering a first treatment dose of 3000 μg/kg of the BCMA×CD3 bispecific antibody; and
subcutaneously administering a first step-up dose of 10 μg/kg of the GPRC5D×CD3 bispecific antibody, then 2-4 days later subcutaneously administering a second step-up dose of 60 μg/kg of the GPRC5D×CD3 bispecific antibody, then 2-4 days later subcutaneously administering a third step-up dose of 400 μg/kg of the GPRC5D×CD3 bispecific antibody, then 2-4 days later subcutaneously administering a first treatment dose of 800 μg/kg of the GPRC5D×CD3 bispecific antibody, and
thereafter, subcutaneously administering the BCMA×CD3 bispecific antibody every two weeks (Q2W) at a treatment dose of 3000 μg/kg and subcutaneously administering the GPRC5D×CD3 bispecific antibody every two weeks (Q2W) at a treatment dose of 800 μg/kg.

In certain embodiments, each treatment dose of the BCMA×CD3 bispecific antibody is administered on the same day as each treatment dose of the GPRC5D×CD3 bispecific antibody.

In certain embodiments, each step-up dose of the BCMA×CD3 bispecific antibody is administered on the same day as each step-up dose of the GPRC5D×CD3 bispecific antibody.

In certain embodiments, the first treatment dose of the BCMA×CD3 bispecific antibody and the first treatment dose of the GPRC5D×CD3 bispecific antibody are administered subcutaneously on Cycle 1 Day 1 of a 28-day cycle, wherein subsequent treatment doses of the GPRC5D×CD3 bispecific antibody are administered Q2W at a dose amount of 800 μg/kg subcutaneously, e.g., on Days 1 and 15 (±3 days) of each 28-day cycle, and subsequent treatment doses of the BCMA×CD3 bispecific antibody are administered Q2W at a dose amount of 3000 μg/kg subcutaneously, e.g., on Days 1 and 15 (±3 days) of a 28-day cycle.

In certain embodiments, the method comprises subcutaneously administering step-up doses of 60 μg/kg and 300 μg/kg and 1500 μg/kg of the BCMA×CD3 bispecific antibody prior to subcutaneously administering said first treatment dose; and
subcutaneously administering step-up doses of 10 μg/kg and 60 μg/kg and 400 μg/kg of the GPRC5D×CD3 bispecific antibody prior to subcutaneously administering said first treatment dose, wherein said first treatment doses are administered 2 to 4 days after administration of the last step-up dose of each bispecific antibody.

In certain embodiments, the method comprises:
subcutaneously administering step-up doses of 60 μg/kg and 300 μg/kg and 1500 μg/kg of the BCMA×CD3 bispecific antibody prior to subcutaneously administering a first treatment dose of the BCMA×CD3 bispecific antibody; and
subcutaneously administering step-up doses of 10 μg/kg and 60 μg/kg and 400 μg/kg of the GPRC5D×CD3 bispecific antibody prior to subcutaneously administering a first treatment dose of the GPRC5D×CD3 bispecific antibody,
wherein the first treatment dose of the BCMA×CD3 bispecific antibody and the first treatment dose of the GPRC5D×CD3 bispecific antibody are subcutaneously administered on Cycle 1 Day 1 of a 28-day cycle,
wherein subsequent treatment doses of the GPRC5D×CD3 bispecific antibody are administered Q2W at a dose amount of 800 μg/kg subcutaneously. e.g., on Days 1 and 15 (±3 days) of each 28-day cycle, and
subsequent treatment doses of the BCMA×CD3 bispecific antibody are administered Q2W at a dose amount of 3000 μg/kg subcutaneously, e.g., on Days 1 and 15 (±3 days) of a 28-day cycle.

In certain embodiments, the method comprises:
subcutaneously administering a first step-up dose of 60 μg/kg of the BCMA×CD3 bispecific antibody, then 2-4 days later subcutaneously administering a second step-up dose of 300 μg/kg of the BCMA×CD3 bispecific antibody, then 2-4 days later subcutaneously administering a third step-up dose of 1500 μg/kg of the BCMA×CD3 bispecific antibody, then 2-4 days later subcutaneously administering a first treatment dose of the BCMA×CD3 bispecific antibody; and
subcutaneously administering a first step-up dose of 10 μg/kg of the GPRC5D×CD3 bispecific antibody, then 2-4 days later subcutaneously administering a second step-up dose of 60 μg/kg of the GPRC5D×CD3 bispecific antibody, then 2-4 days later subcutaneously administering a third step-up dose of 400 μg/kg of the GPRC5D×CD3 bispecific antibody, then 2-4 days later subcutaneously administering a first treatment dose of the GPRC5D×CD3 bispecific antibody,
wherein the first treatment dose of the BCMA×CD3 bispecific antibody and the first treatment dose of the GPRC5D×CD3 bispecific antibody are subcutaneously administered on Cycle 1 Day 1 of a 28-day cycle,
wherein subsequent treatment doses of the GPRC5D×CD3 bispecific antibody are administered Q2W at a dose amount of 800 μg/kg subcutaneously, e.g., on Days 1 and 15 (±3 days) of each 28-day cycle, and
subsequent treatment doses of the BCMA×CD3 bispecific antibody are administered Q2W at a dose amount of 3000 μg/kg subcutaneously. e.g., on Days 1 and 15 (±3 days) of a 28-day cycle.

In certain embodiments, the method comprises:
subcutaneously administering step-up doses of 60 μg/kg and 300 μg/kg and 1500 μg/kg of the BCMA×CD3 bispecific antibody prior to subcutaneously administering a first treatment dose of the BCMA×CD3 bispecific antibody, and
subcutaneously administering step-up doses of 10 μg/kg and 60 μg/kg and 300 μg/kg of the GPRC5D×CD3 bispecific antibody prior to subcutaneously administering a first treatment dose of the GPRC5D×CD3 bispecific antibody,
wherein the first treatment dose of the BCMA×CD3 bispecific antibody and the first treatment dose of the GPRC5D×CD3 bispecific antibody are subcutaneously administered on Cycle 1 Day 1 of a 28-day cycle,
wherein subsequent treatment doses of the GPRC5D×CD3 bispecific antibody are administered Q2W at a dose amount of 800 μg/kg subcutaneously. e.g., on Days 1 and 15 (±3 days) of each 28-day cycle, and
subsequent treatment doses of the BCMA×CD3 bispecific antibody are administered Q2W at a dose amount of 3000 μg/kg subcutaneously, e.g., on Days 1 and 15 (±3 days) of a 28-day cycle.

In certain embodiments, the method comprises:
subcutaneously administering a first step-up dose of 60 μg/kg of the BCMA×CD3 bispecific antibody, then 2-4 days later subcutaneously administering a second step-up dose of 300 μg/kg of the BCMA×CD3 bispecific antibody, then 2-4 days later subcutaneously administering a third step-up dose of 1500 μg/kg of the BCMA×CD3 bispecific antibody, then 2-4 days later subcutaneously administering a first treatment dose of the BCMA×CD3 bispecific antibody; and
subcutaneously administering a first step-up dose of 10 μg/kg of the GPRC5D×CD3 bispecific antibody, then 2-4 days later subcutaneously administering a second step-up dose of 60 μg/kg of the GPRC5D×CD3 bispecific antibody, then 2-4 days later subcutaneously administering a third step-up dose of 300 μg/kg of the GPRC5D×CD3 bispecific antibody, then 2-4 days later subcutaneously administering a first treatment dose of the GPRC5D×CD3 bispecific antibody,
wherein the first treatment dose of the BCMA×CD3 bispecific antibody and the first treatment dose of the GPRC5D×CD3 bispecific antibody are subcutaneously administered on Cycle 1 Day 1 of a 28-day cycle,
wherein subsequent treatment doses of the GPRC5D×CD3 bispecific antibody are administered Q2W at a dose amount of 800 μg/kg subcutaneously, e.g., on Days 1 and 15 (±3 days) of each 28-day cycle, and
subsequent treatment doses of the BCMA×CD3 bispecific antibody are administered Q2W at a dose amount of 3000 μg/kg subcutaneously, e.g., on Days 1 and 15 (±3 days) of a 28-day cycle.

In certain embodiments, the method achieves a partial response, a very good partial response, a complete response or a stringent complete response in the subject, as determined by IMWG response criteria.

In certain embodiments, the method achieves an overall response rate of at least 50% in a population of subjects with relapsed or refractory multiple myeloma (RRMM), e.g., in subjects with EMD.

In certain embodiments, the method achieves an overall response rate of at least 55% in a population of subjects with relapsed or refractory multiple myeloma (RRMM), e.g., in subjects with EMD.

In certain embodiments, the method achieves an overall response rate of at least 60% in a population of subjects with relapsed or refractory multiple myeloma (RRMM), e.g., in subjects with EMD.

In certain embodiments, the method achieves an overall response rate of at least 65% in a population of subjects with relapsed or refractory multiple myeloma (RRMM), e.g., in subjects with EMD.

In certain embodiments, the method achieves an overall response rate of at least 70% in a population of subjects with relapsed or refractory multiple myeloma (RRMM), e.g., in subjects with EMD.

In certain embodiments, the method achieves an overall response rate of at least 80% in a population of subjects with RRMM, e.g., in subjects with EMD.

In certain embodiments, the method achieves an overall response rate of at least 85% in a population of subjects with RRMM, e.g., in subjects with EMD.

In certain embodiments, the method achieves an overall response rate of at least 90% in a population of subjects with RRMM.

In certain embodiments, the method achieves an overall response rate of at least 95% in a population of subjects with RRMM.

In certain embodiments, the method achieves a complete response or a stringent complete response rate of at least 20% in a population of subjects with RRMM, e.g., in subjects with EMD.

In certain embodiments, the method achieves a complete response or a stringent complete response rate of at least 25% in a population of subjects with RRMM, e.g., in subjects with EMD.

In certain embodiments, the method achieves a complete response or a stringent complete response rate of at least 30% in a population of subjects with RRMM, e.g., in subjects with EMD.

In certain embodiments, the method achieves a complete response or a stringent complete response rate of at least 35% in a population of subjects with RRMM, e.g., in subjects with EMD.

In certain embodiments, the method achieves a complete response or a stringent complete response rate of at least 40% in a population of subjects with RRMM. e.g., in subjects with EMD.

All methods described herein, however expressed, may be described as corresponding uses, in particular medical uses.

### BRIEF DESCRIPTION OF THE FIGURES

The following figures form part of the present specification and are included to further demonstrate certain aspects of the present invention. The invention may be better understood by reference to one or more of these figures in combination with the description of specific embodiments presented herein.

FIG. 1 provides a Phase 1b Clinical Study Design of the Phase 1b RedirecTT-1 trial (NCT04586426) with Teclistamab (Tec) and Talquetamab (Tal) Simultaneously Targeting BCMA and GPRC5D in Patients with Relapsed/Refractory Multiple Myeloma (RRMM).

FIG. 2 provides patient characteristics of the Phase 1b RedirecTT-1 trial (NCT04586426), as of Mar. 16, 2023.

FIG. 3 provides safety profile information of the Phase 1b RedirecTT-1 trial (NCT04586426), as of Mar. 16, 2023.

FIG. 4 provides safety profile information of the Phase 1b RedirecTT-1 trial (NCT04586426), as of Mar. 16, 2023.

FIG. 5 provides safety profile information of the Phase 1b RedirecTT-1 trial (NCT04586426), as of Mar. 16, 2023.

FIG. 6 provides safety profile information of the Phase 1b RedirecTT-1 trial (NCT04586426), as of Mar. 16, 2023.

FIG. 7 provides safety profile information of the Phase 1b RedirecTT-1 trial (NCT04586426), as of Mar. 16, 2023.

FIG. 8 provides efficacy results from the Phase 1b RedirecTT-1 trial (NCT04586426), as of Mar. 16, 2023.

FIG. 9 provides efficacy results for patients in the EMD subgroup from the Phase 1b RedirecTT-1 trial (NCT04586426), as of Mar. 16, 2023.

FIG. 10 provides a Clinical Study Design of Part 3 of the RedirecTT-1 trial (Phase 2).

FIG. 11 provides an illustration of the dosing regimen for Arm B (Tal+Tec) of the MonumenTAL-6 clinical trial (Phase 3).

### DETAILED DESCRIPTION OF THE INVENTION

The disclosed methods can be understood more readily by reference to the following detailed description. It is to be understood that the disclosed methods are not limited to the specific methods described and/or shown herein, and that the terminology used herein is for the purpose of describing particular embodiments by way of example only and is not intended to be limiting of the claimed methods. All patents, published patent applications and publications cited herein are incorporated by reference as if set fourth fully herein.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

“About” when used in reference to numerical ranges, cutoffs, or specific values means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of an assay, result or embodiment. “about” means within one standard deviation per the practice in the art, or a range of up to 5%, whichever is larger.

“Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g. IgM). Each heavy chain is comprised of a heavy chain variable region (VH) and a heavy chain constant region (comprised of domains CHL, hinge, CH2 and CH3). Each light chain is comprised of a light chain variable region (VL) and a light chain constant region (CL). The VH and the VL regions can be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins can be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species can be assigned to one of two clearly distinct types, namely kappa (u) and lambda (k), based on the amino acid sequences of their constant domains.

“Antigen binding fragment” or “antigen binding domain” refers to a portion of an immunoglobulin molecule that binds an antigen. Antigen binding fragments can be synthetic, enzymatically obtainable or genetically engineered polypeptides and include the VH, the VL, the VH and the VL, Fab, F(ab′)2, Fd and Fv fragments, domain antibodies (dAb) consisting of one VH domain or one VL domain, shark variable IgNAR domains, camelized VH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3. VH and VL domains can be linked together via a synthetic linker to form various types of single chain antibody designs where the VH/VL domains can pair intramolecularly, or intermolecularly in those cases when the VH and VL domains are expressed by separate single chain antibody constructs, to form a monovalent antigen binding site, such as single chain Fv (scFv) or diabody; described for example in Int. Patent Publ. Nos. WO1998/44001, WO1988/01649, WO1994/13804 and WO1992/01047.

“BCMA” refers to human B-cell maturation antigen, also known as CD269 or TNFRSF17 (UniProt Q02223). The extracellular domain of BCMA encompasses residues 1-54 of Q02223. Human BCMA comprises the amino acid sequence of SEQ ID NO: 1.

SEQ ID NO: 1
MLQMAGQCSQNEYFDSLLHACIPCQLRCSSNTPPLTCQRYCNASVTNSVK
GTNAILWTCLGLSLIISLAVFVLMFLLRKINSEPLKDEFKNTGSGLLGMA
NIDLEKSRTGDEIILPRGLEYTVEECTCEDCIKSKPKVDSDHCFPLPAME
EGATILVTTKTNDYCKSLPAALSATEIEKSISAR

“Bispecific” refers to an antibody that specifically binds two distinct antigens or two distinct epitopes within the same antigen. The bispecific antibody can have cross-reactivity to other related antigens, for example to the same antigen from other species (homologs), such as human or monkey, for example Macaca cynomolgus (cynomolgus, cyno) or Pan troglodytes, or can bind an epitope that is shared between two or more distinct antigens.

“BCMA×CD3 bispecific antibody” refers to a bispecific antibody that specifically binds BCMA and CD3.

“Cancer” refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. A “cancer” or “cancer tissue” can include a tumor.

“CD3” refers to a human antigen which is expressed on T cells as part of the multimolecular T cell receptor (TCR) complex and which consists of a homodimer or heterodimer formed from the association of two or four receptor chains: CD3 epsilon, CD3 delta, CD3 zeta and CD3 gamma. Human CD3 epsilon comprises the amino acid sequence of SEQ ID NO: 2. SEQ ID NO: 3 shows the extracellular domain of CD3 epsilon.

SEQ ID NO: 2
MQSGTHWRVLGLCLLSVGVWGQDGNEEMGGITQTPYKVSISGTTVILTCP
QYPGSEILWQHNDKNIGGDEDDKNIGSDEDHLSLKEFSELEQSGYYVCYP
RGSKPEDANFYLYLRARVCENCMEMDVMSVATIVIVDICITGGLLLLVYY
WSKNRKAKAKPVTRGAGAGGRQRGQNKERPPPVPNPDYEPIRKGQRDLYS
GLNQRRI
SEQ ID NO: 3
DGNEEMGGITQTPYKVSISGTTVILTCPQYPGSEILWQHNDKNIGGDEDD
KNIGSDEDHLSLKEFSELEQSGYYVCYPRGSKPEDANFYLYLRARVCENC
MEMD

“CH3 region” or “CH3 domain” refers to the CH3 region of an immunoglobulin. The CH3 region of human IgG1 antibody corresponds to amino acid residues 341-446. However, the CH3 region can also be any of the other antibody isotypes as described herein.

“Combination” means that two or more therapeutics are administered to a subject together in a mixture, concurrently as single agents or sequentially as single agents in any order.

“Combination dosing regimen” (also referred to herein as “combination regimen”) means that two or more therapeutics are administered to a subject on each therapeutic's respective dosing schedule over a time period (e.g., a time period may comprise one or more treatment cycles, such as one or more 28-day treatment cycles); for example, a combination dosing regimen may comprise administering to a subject (i) “therapeutic #1” on its weekly or bi-weekly or monthly dosing schedule starting on Day 1 of a treatment cycle and (ii) “therapeutic #2” on its weekly or bi-weekly or monthly dosing schedule starting on Day 1 of the same treatment cycle. It is contemplated that in certain embodiments “therapeutic #1” and “therapeutic #2” are administered on the same day throughout the combination dosing regimen because they have the same or similar dosing schedules (e.g., both have a bi-weekly dosing schedule and treatment starts on the same day).

“Complementarity determining regions” (CDR) are antibody regions that bind an antigen. CDRs can be defined using various delineations such as Kabat (Wu et al. J Exp Med 132; 211-50, 1970) (Kabat et al., Sequences of Proteins of Immunological Interest, 5th Ed. Public Health Service, National Institutes of Health, Bethesda, Md., 1991), Chothia (Chothia et al. J Mol Biol 196: 901-17, 1987), IMGT (Lefranc et al. Dev Comp Immunol 27: 55-77, 2003) and AbM (Martin and Thornton J Bmol Biol 263: 8(0)-15, 1996). The correspondence between the various delineations and variable region numbering are described (see e.g. Lefranc et al. Dev Comp Immunol 27: 55-77, 2003; Honegger and Pluckthun, J Mol Biol 309:657-70, 2001; International ImMunoGeneTics (IMGT) database; Web resources, http://www_imgt_org). Available programs such as abYsis by UCL Business PLC can be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification. Preferably, the term “CDR”, “HCDR1”, “HCDR2”, “HCDR3”, “LCDR1”, “LCDR2” and “LCDR3” as used herein includes CDRs defined by the method of Kabat.

“Comprising” is intended to include examples encompassed by the terms “consisting essentially of” and “consisting of”; similarly, the term “consisting essentially of” is intended to include examples encompassed by the term “consisting of.” Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

“Fe gamma receptor” (FcγR) refers to well-known FcγRI, FcγRIIa, FcγRIIb or FcγRIII. Activating FcγR includes FcγRI, FcγRIIa and FcγRIII.

“GPRC5D×CD3 bispecific antibody” refers to a bispecific antibody that specifically binds GPRC5D and CD3.

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs. or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” can contain consensus framework sequences derived from human framework sequence analyses, for example as described in Knappik et al., (2000) J Mol Biol 296:57-86, or synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

“Humanized antibody” refers to an antibody in which at least one CDR is derived from non-human species and at least one framework is derived from human immunoglobulin sequences. Humanized antibody can include substitutions in the frameworks so that the frameworks can not be exact copies of expressed human immunoglobulin or human immunoglobulin germline gene sequences.

“Identity” refers to a relationship between the sequences of two or more polypeptide molecules or two or more nucleic acid molecules, as determined by aligning and comparing the sequences. “Percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN (DNAStar. Inc.) software. Those skilled in the art can determine appropriate parameters for aligning sequences, including any algorithms needed to achieve maximal alignment over the full length of the sequences being compared.

“Isolated” refers to a homogenous population of molecules (such as synthetic polynucleotides or a protein such as an antibody) which have been substantially separated and/or purified away from other components of the system the molecules are produced in, such as a recombinant cell, as well as a protein that has been subjected to at least one purification or isolation step. “Isolated antibody” refers to an antibody that is substantially free of other cellular material and/or chemicals and encompasses antibodies that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deanudation, methionine oxidation or asparagine or glutamine deamidation. Monoclonal antibodies typically bind one antigenic epitope. A bispecific monoclonal antibody binds two distinct antigenic epitopes. Monoclonal antibodies can have heterogeneous glycosylation within the antibody population. Monoclonal antibody can be monospecific or multispecific such as bispecific, monovalent, bivalent or multivalent.

“Mutation” refers to an engineered or naturally occurring alteration in a polypeptide or polynucleotide sequence when compared to a reference sequence. The alteration can be a substitution, insertion or deletion of one or more amino acids or polynucleotides.

“Negative minimal residual disease status” or “negative MRD status” or “MRD negative” refers to the PerMillionCount (i.e., a point estimate of malignant myeloma cells per million nucleated cells) in a patients on-study bone marrow sample relative to their reference bone marrow sample (i.e., Teclistamab treatment naïve bone marrow sample). Based on this PerMillionCount, each sample is determined to be positive or negative. Samples are positive if the PerMillionCount is greater than or equal to the limit of sensitivity, otherwise they are negative. Negative minimal residual disease status can be determined at a sensitivity of 0.01% (104), 0.001% (10-s) or 0.0001% (10⁻⁶). Negative minimal residual disease status was determined using next generation sequencing (NGS).

“Pharmaceutical composition” refers to composition that comprises an active ingredient and a pharmaceutically acceptable carrier.

“Pharmaceutically acceptable carrier” or “excipient” refers to an ingredient in a pharmaceutical composition, other than the active ingredient, which is nontoxic to a subject.

“Recombinant” refers to DNA, antibodies and other proteins that are prepared, expressed, created or isolated by recombinant means when segments from different sources are joined to produce recombinant DNA, antibodies or proteins.

“Refractory” refers to a cancer that is not amendable to surgical intervention and is initially unresponsive to therapy.

“Relapsed” refers to a cancer that responded to treatment but then returns.

“Step-up dose” refers to a dose of an active agent that is administered to a subject prior to a treatment dose. A step-up dose is lower than the treatment dose. To prevent or lessen certain toxicities, such as cytokine release syndrome (CRS), a “priming” dose strategy may include one or more lower step-up dose(s) followed by higher treatment doses. A “step-up phase” refers to an initial phase of a therapeutically effective regimen in which at least one step-up dose of a therapeutic is administered to the subject. A step-up phase may also include one or more treatment doses, i.e., a step-up phase may include one or more step-up doses followed by one or more treatment doses; for example, a step-up phase may include two step-up doses followed by two treatment doses, or three step-up doses followed by one treatment dose. In particular embodiments, the step-up phase is 28 days, i.e., the step-up phase is a 28-day cycle of a therapeutically effective regimen. According to certain embodiments, a step-up phase occurs in treatment Cycle 1 of a therapeutic regimen comprising sequential treatment cycles.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. Except when noted, the terms “patient” or “subject” are used interchangeably.

“T cell redirecting therapeutic” refers to a molecule containing two or more binding regions, wherein one of the binding regions specifically binds a cell surface antigen on a target cell or tissue and wherein a second binding region of the molecule specifically binds a T cell antigen. Examples of cell surface antigen include a tumor associated antigen, such as BCMA. Examples of T cell antigen include, e.g., CD3. This dual/multi-target binding ability recruits T cells to the target cell or tissue leading to the eradication of the target cell or tissue.

“Therapeutically effective amount” refers to an amount effective, at doses and for periods of time necessary, to achieve a desired therapeutic result. A therapeutically effective amount can vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient.

“Treat” or “treatment” refers to both therapeutic treatment and prophylactic or preventative measures, wherein the object is to prevent or slow down (lessen) an undesired physiological change or disorder. Beneficial or desired clinical results include alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if a subject was not receiving treatment. Those in need of treatment include those already with the condition or disorder as well as those prone to have the condition or disorder or those in which the condition or disorder is to be prevented.

“Treatment dose”—refers to a dose of the active agent that is administered to a subject to treat a disease. A treatment dose may be administered at a regular dosing interval on a repetitive basis (e.g. weekly, biweekly, monthly). A treatment dose may be preceded by one or more step-up doses.

A patient that is “triple-class exposed” refers to a patient diagnosed with multiple myeloma (MM) that has previously been treated with (at a minimum) a proteasome inhibitor, an immunomodulatory agent and an anti-CD38 monoclonal antibody.

“Tumor cell” or a “cancer cell” refers to a cancerous, pre-cancerous or transformed cell, either in vivo, ex vivo, or in tissue culture, that has spontaneous or induced phenotypic changes. These changes do not necessarily involve the uptake of new genetic material. Although transformation can arise from infection with a transforming virus and incorporation of new genomic nucleic acid, uptake of exogenous nucleic acid or it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo.

The numbering of amino acid residues in the antibody constant region throughout the specification is according to the EU index as described in Kabat et al., Sequences of Proteins of Immunological Interest, 5th Ed. Public Health Service, National Institutes of Health. Bethesda, MD. (1991), unless otherwise explicitly stated. Antibody constant chain numbering can be found for example at ImMunoGeneTics website, at IMGT Web resources at IMGT Scientific charts.

Conventional one and three-letter amino acid codes are used herein as shown in Table 1.

TABLE 1

| Amino acid | Three-letter code | One-letter code |
|---|---|---|
| Alanine | Ala | A |
| Arginine | Arg | R |
| Asparagine | Asn | N |
| Aspartate | Asp | D |
| Cysteine | Cys | C |
| Glutamate | Gln | E |
| Glutamine | Glu | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

### BCMA×CD3 and GPRC5D×CD3 Bispecific Antibodies and Uses Thereof

In patients with relapsed or refractory disease who have exhausted several therapies, multiple myeloma remains an incurable malignancy and an unmet medical need with significant morbidity and mortality. There remains a need for new dosing regimens that provide improved safety profiles and enhanced efficacy.

The present inventors have developed novel combination regimens which target two distinct antigens and overcome challenges associated with combining two therapeutics. Described herein are combination regimens comprising BCMA- and GPRC5D-targeted bispecific antibodies. Also described herein are clinical results from the first ever BCMA- and GPRC5D-targeted combination study, in which patients were treated with combination regimens of teclistamab (a BCMA×CD3 bispecific antibody) and talquetamab (a GPRC5D×CD3 bispecific antibody).

For novel combination regimens involving two therapeutics, there is a known risk of overlapping toxicity profiles. For example, cytokine release syndrome (CRS) commonly occurs following administration of immunotherapies. Patients with RRMM are already at increased risk of infection, and bispecific antibodies targeting B-cell maturation antigen (BCMA) may contribute to increased infection risk, due to on-target, off-tumor toxicity. In the phase 1/2 MajesTEC-1 clinical study of teclistamab (NCT03145181 and NCT04557098), patients (N=165) received subcutaneous teclistamab 1.5 mg/kg weekly following a step-up dosing schedule (0.06 mg/kg and 0.3 mg/kg, each separated by 2-4 days). At a median follow-up of 21.6 months (range 0.26-32.69), infections were reported in 129 patients (78.2%). Overall, grade 3/4 infections occurred in 86 patients (52.1%), most commonly pneumonia (20.6%), COVID-19 (18.8%), sepsis (6.1%), and urinary tract infection (6.1%). Grade 3/4 neutropenia occurred in 65.5% of patients.

Given that BCMA×CD3 and GPRC5D×CD3 bispecific antibodies act by stimulating the endogenous immune system, there is the potential for toxicity on other tissues or organs by activating immune cells through a potential inflammatory mechanism. Despite these challenges, the inventors have developed combination regimens with high efficacy that do not exhibit additive toxicity. Without being bound by theory, targeting two distinct antigens found on multiple myeloma cells may overcome common mechanisms of resistance to monotherapy treatment by reducing the risk of loss of activity due to single target antigen loss and enhancing antigen-antibody interaction; therefore, dual targeting may also mitigate target molecule-related escape, potentially reducing the risk of relapse.

For patients whose myeloma relapses after exposure to daratumumab and lenalidomide in the first or subsequent lines of therapy, treatment options are limited. In a retrospective review of patients who were refractory to anti-CD38 therapy and who received subsequent therapy after progression on PI, IMiD, and anti-CD38 therapy, the observed efficacy of the next treatment after was dismal, regardless of the salvage therapy chosen. Therefore, there remains a significant and critical unmet need for new therapeutic options directed at alternative mechanisms of action that can better control the disease, provide deeper, more sustained responses, and better long-term outcomes including maintenance of Health-related Quality of Life (HRQoL).

According to certain embodiments, combination regimens of the present invention comprising BCMA×CD3 and GPRC5D×CD3 bispecific antibodies (e.g., regimens comprising Teclistamab and Talquetamab) improve median progression free survival (PFS) in a population of subjects with relapsed or refractory multiple myeloma that have received between 1-4 prior lines of therapy including an anti-CD38 antibody (e.g., daratumumab) and lenalidomide, wherein the improvement in median PFS is relative to median PFS of a reference population of subjects with relapsed or refractory multiple myeloma that have received between 1-4 prior lines of therapy including an anti-CD38 antibody (e.g., daratumumab) and lenalidomide, said reference population having been administered either (i) elotuzumab, pomalidomide, and dexamethasone (EPd) or (ii) pomalidomide, bortezomib, and dexamethasone (PVd). PFS refers to the duration from the date of treatment start to either progressive disease or death, whichever comes first.

According to certain embodiments, combination regimens of the present invention comprising BCMA×CD3 and GPRC5D×CD3 bispecific antibodies (e.g., regimens comprising Teclistamab and Talquetamab) improve overall response rate (ORR) in a population of subjects with relapsed or refractory multiple myeloma that have received between 1-4 prior lines of therapy including an anti-CD38 antibody (e.g., daratumumab) and lenalidomide, wherein the improvement in ORR is relative to ORR of a reference population of subjects with relapsed or refractory multiple myeloma that have received between 1-4 prior lines of therapy including an anti-CD38 antibody (e.g., daratumumab) and lenalidomide, said reference population having been administered either (i) elotuzumab, pomalidomide, and dexamethasone (EPd) or (ii) pomalidomide, bortezomib, and dexamethasone (PVd). ORR refers to the percentage of subjects with best overall response of partial response (PR) or better according to international myeloma working group (IMWG) response criteria.

According to certain embodiments, combination regimens of the present invention comprising BCMA×CD3 and GPRC5D×CD3 bispecific antibodies (e.g., regimens comprising Teclistamab and Talquetamab) improve Complete Response (CR) or Better Rate in a population of subjects with relapsed or refractory multiple myeloma that have received between 1-4 prior lines of therapy including an anti-CD38 antibody (e.g., daratumumab) and lenalidomide, wherein the improvement in CR or Better Rate is relative to CR or Better Rate of a reference population of subjects with relapsed or refractory multiple myeloma that have received between 1-4 prior lines of therapy including an anti-CD38 antibody (e.g., daratumumab) and lenalidomide, said reference population having been administered either (i) elotuzumab, pomalidomide, and dexamethasone (EPd) or (ii) pomalidomide, bortezomib, and dexamethasone (PVd). CR or better rate refers to the percentage of subjects with best overall response of CR or better according to IMWG response criteria.

According to certain embodiments, combination regimens of the present invention comprising BCMA×CD3 and GPRC5D×CD3 bispecific antibodies (e.g., regimens comprising Teclistamab and Talquetamab) improve Very Good Partial Response (VGPR) or Better Rate in a population of subjects with relapsed or refractory multiple myeloma that have received between 1-4 prior lines of therapy including an anti-CD38 antibody (e.g., daratumumab) and lenalidomide, wherein the improvement in VGPR or Better Rate is relative to VGPR or Better Rate of a reference population of subjects with relapsed or refractory multiple myeloma that have received between 1-4 prior lines of therapy including an anti-CD38 antibody (e.g., daratumumab) and lenalidomide, said reference population having been administered either (i) elotuzumab, pomalidomide, and dexamethasone (EPd) or (ii) pomalidomide, bortezomib, and dexamethasone (PVd). VGPR or better rate refers to the percentage of subjects with best overall response of VGPR or better according to IMWG response criteria.

According to certain embodiments, combination regimens of the present invention comprising BCMA×CD3 and GPRC5D×CD3 bispecific antibodies (e.g., regimens comprising Teclistamab and Talquetamab) improve Minimal Residual Disease (MRD)-negative CR Rate in a population of subjects with relapsed or refractory multiple myeloma that have received between 1-4 prior lines of therapy including an anti-CD38 antibody (e.g., daratumumab) and lenalidomide, wherein the improvement in MRD-negative CR Rate is relative to MRD-negative CR Rate of a reference population of subjects with relapsed or refractory multiple myeloma that have received between 1-4 prior lines of therapy including an anti-CD38 antibody (e.g., daratumumab) and lenalidomide, said reference population having been administered either (i) elotuzumab, pomalidomide, and dexamethasone (EPd) or (ii) pomalidomide, bortezomib, and dexamethasone (PVd). MRD-negative CR refers to the percentage of subjects who achieve both CR or better and MRD negativity at a threshold of 10\hat{ }−5 at any timepoint after the date of randomization and before disease progression or start of subsequent antimyeloma therapy (SST).

According to certain embodiments, combination regimens of the present invention comprising BCMA×CD3 and GPRC5D×CD3 bispecific antibodies (e.g., regimens comprising Teclistamab and Talquetamab) improve overall survival (OS) in a population of subjects with relapsed or refractory multiple myeloma that have received between 1-4 prior lines of therapy including an anti-CD38 antibody (e.g., daratumumab) and lenalidomide, wherein the improvement in OS is relative to OS of a reference population of subjects with relapsed or refractory multiple myeloma that have received between 1-4 prior lines of therapy including an anti-CD38 antibody (e.g., daratumumab) and lenalidomide, said reference population having been administered either (i) elotuzumab, pomalidomide, and dexamethasone (EPd) or (ii) pomalidomide, bortezomib, and dexamethasone (PVd). OS refers to time from start of treatment to the date of the subject's death.

### Antibodies of the Present Invention

Any suitable BCMA×CD3 bispecific antibody known to those skilled in the art in view of the present disclosure can be used in the invention. Any suitable GPRC5D×CD3 bispecific antibody known to those skilled in the art in view of the present disclosure can be used in the invention.

Various bispecific antibody formats include formats described herein and recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together, ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule, or bispecific antibodies generated by arm exchange. Exemplary bispecific formats include dual targeting molecules include Dual Targeting (DT)-Ig (GSK/Domantis), Two-in-one Antibody (Genentech) and mAb2 (F-Star), Dual Variable Domain (DVD)-Ig (Abbott). DuoBody (Genmab), Ts2Ab (MedImmune/AZ) and BsAb (Zymogenetics), HERCULES (Biogen Idec) and TvAb (Roche), ScFv/Fc Fusions (Academic Institution), SCORPION (Emergent BioSolutions/Trubion, Zymogenetics/BMS) and Dual Affinity Retargeting Technology (Fc-DART) (MacroGenics), F(ab)2 (Medarex/AMGEN), Dual-Action or Bis-Fab (Genentech), Dock-and-Lock (DNL) (ImmunoMedics), Bivalent Bispecific (Biotecnol) and Fab-Fv (UCB-Celltech), Bispecific T Cell Engager (BITE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies. Various formats of bispecific antibodies have been described, for example in Chames and Baty (2009) Curr Opin Drug Disc Dev 12: 276 and in Nunez-Prado et al., (2015) Drug Discovery Today 20(5):588-594.

In some embodiments, the BCMA×CD3 bispecific antibody comprises any one of the BCMA binding domains described in WO2017/031104, the entire content of which is incorporated herein by reference. In some embodiments, the BCMA×CD3 bispecific antibody comprises any one of the CD3 binding domains described in WO2017/031104.

In some embodiments, the BCMA×CD3 bispecific antibody comprises any one of the BCMA×CD3 bispecific antibodies described in WO2017/031104.

In some embodiments, the GPRC5D×CD3 bispecific antibody comprises any one of the GPRC5D binding domains described in U.S. Pat. No. 10,562,968, the content of which is incorporated herein by reference in its entirety. In some embodiments, the GPRC5D×CD3 bispecific antibody comprises any one of the CD3 binding domains described in U.S. Pat. No. 10,562,968. In some embodiments, the GPRC5D×CD3 bispecific antibody comprises any one of the GPRC5D×CD3 bispecific antibodies described in U.S. Pat. No. 10,562,968.

In some embodiments, the BCMA×CD3 bispecific antibody is chimeric, humanized or human.

In some embodiments, the GPRC5D×CD3 bispecific antibody is chimeric, humanized or human.

In some embodiments, the bispecific antibody is an IgG1, an IgG2, an IgG3 or an IgG4 isotype. In preferred embodiments, the bispecific antibody is an IgG4 isotype. An exemplary wild-type IgG4 comprises an amino acid sequence of SEQ ID NO: 34.

SEQ ID NO: 34:
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRW
QEGNVFSCSVMHEALHNHYTQKSLSLSLGK

The bispecific antibody can be of any allotype. It is expected that allotype has no influence on properties of the bispecific antibodies, such as binding or Fc-mediated effector functions. Immunogenicity of therapeutic antibodies is associated with increased risk of infusion reactions and decreased duration of therapeutic response (Baert et al., (2003) N Engl J Med 348:602-08). The extent to which therapeutic antibodies induce an immune response in the host can be determined in part by the allotype of the antibody (Stickler et al., (2011) Genes and Immunity 12:213-21). Antibody allotype is related to amino acid sequence variations at specific locations in the constant region sequences of the antibody. Table 2 shows select IgG1, IgG2 and IgG4 allotypes.

TABLE 2

| | Amino acid residue at position of diversity | | | | | | | |
|---|---|---|---|---|---|---|---|---|
| | (residue numbering: EU Index) | | | | | | | |
| | IgG2 | | IgG4 | | IgG1 | | | |
| Allotype | 189 | 282 | 309 | 422 | 214 | 356 | 358 | 431 |
| G2m(n) | T | M | | | | | | |
| G2m(n-) | P | V | | | | | | |
| G2m(n)/(n- | T | V | | | | | | |
| nG4m(a) | | | L | R | | | | |
| G1m(17) | | | | | K | E | M | A |
| G1m(17, 1) | | | | | K | D | L | A |

In some embodiments, the bispecific antibody comprises one or more Fc substitutions that reduces binding of the bispecific antibody to a Fcγ receptor (FcγR) and/or reduces Fc effector functions such as C1q binding, complement dependent cytotoxicity (CDC), antibody-dependent cell-mediated cytotoxicity (ADCC) or phagocytosis (ADCP). The specific substitutions can be made in comparison to the wild-type IgG4 of SEQ ID NO: 34.

Fc positions that can be substituted to reduce binding of the Fc to the activating FcγR and subsequently to reduce effector function are substitutions L234A/L235A on IgG1, V234A/G237A/P238S/H268A/V309L/A330S/P331S on IgG2, F234A/L235A on IgG4, S228P/F234A/L235A on IgG4, N297A on all Ig isotypes. V234A/G237A on IgG2, K214T/E233P/L234V/L235A/G236-deleted/A327G/P331A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4, and S228P/F234A/L235A/G236-deleted/G237A/P238S on IgG4, wherein residue numbering is according to the EU index.

Fc substitutions that can be used to reduce CDC are a K322A substitution.

Well-known S228P substitution can further be made in IgG4 antibodies to enhance IgG4 stability.

In some embodiments, the bispecific antibody comprises one or more asymmetric substitutions in a first CH3 domain or in a second CH3 domain, or in both the first CH3 domain and the second CH3 domain.

In some embodiments, the one or more asymmetric substitutions is selected from the group consisting of F405L/K409R, wild-type/F405L_R409K, T366Y/F405A, T366W/F405W, F405W/Y407A, T394W/Y407T, T394S/Y407A, T366W/T394S, F405W/T394S and T366W/T366S_L368A_Y407V, L351Y_F405A_Y407V/T394W, T3661_K392M_T394W/F405A_Y407V, T366L_K392M_T394W/F405A_Y407V, L351Y_Y407A/T366A_K409F, L351Y_Y407A/T366V_K409F, Y407A/T366A_K409F and T350V_L351Y_F405A_Y407V/T350V_T366L_K392L_T394W.

In some embodiments, the BCMA×CD3 bispecific antibody is an IgG4 isotype and comprises phenylalanine at position 405 and arginine at position 409 in a first heavy chain (HC1) and leucine at position 405 and lysine at position 40) in a second heavy chain (HC2), wherein residue numbering is according to the EU Index.

In some embodiments, the GPRC5D×CD3 bispecific antibody is an IgG4 isotype and comprises phenylalanine at position 405 and arginine at position 409 in a first heavy chain (HC1) and leucine at position 405 and lysine at position 409 in a second heavy chain (HC2), wherein residue numbering is according to the EU Index.

In some embodiments, the BCMA×CD3 bispecific antibody further comprises proline at position 228, alanine at position 234 and alanine at position 235 in both the HC1 and the HC2.

In some embodiments, the GPRC5D×CD3 bispecific antibody further comprises proline at position 228, alanine at position 234 and alanine at position 235 in both the HC1 and the HC2.

Tables 3 and 4 provide sequences of an exemplary embodiment of a BCMA×CD3 bispecific antibody, according to the Kabat numbering system.

TABLE 3
Sequences of BCMA binding arm

| | | SEQ ID |
|---|---|---|
| Region | Sequence | NO: |
| HCDR1 | SGSYFWG | 4 |
| HCDR2 | SIYYSGITYYNPSLKS | 5 |
| HCDR3 | HDGAVAGLFDY | 6 |
| LCDR1 | GGNNIGSKSVH | 7 |
| LCDR2 | DDSDRPS | 8 |
| LCDR3 | QVWDSSSDHVV | 9 |
| VH | QLQLQESGPGLVKPSETLSLTCTVSGGSISSGSY | 10 |
| | FWGWIRQPPGKGLEWIGSIYYSGITYYNPSLKS | |
| | RVTISVDTSKNQFSLKLSSVTAADTAVYYCAR | |
| | HDGAVAGLFDYWGQGTLVTVSS | |
| VL | SYVLTQPPSVSVAPGQTARITCGGNNIGSKSVH | 11 |
| | WYQQPPGQAPVVVVYDDSDRPSGIPERFSGSN | |
| | SGNTATLTISRVEAGDEAVYYCQVWDSSSDHV | |
| | VFGGGTKLTVL | |
| HC | QLQLQESGPGLVKPSETLSLTCTVSGGSISSGSY | 12 |
| | FWGWIRQPPGKGLEWIGSIYYSGITYYNPSLKS | |
| | RVTISVDTSKNQFSLKLSSVTAADTAVYYCAR | |
| | HDGAVAGLFDYWGQGTLVTVSSASTKGPSVFP | |
| | LAPCSRSTSESTAALGCLVKDYFPEPVTVSWNS | |
| | GALTSGVHTFPAVLQSSGLYSLSSVVTVPSSSL | |
| | GTKTYTCNVDHKPSNTKVDKRVESKYGPPCPP | |
| | CPAPEAAGGPSVFLFPPKPKDTLMISRTPEVTCV | |
| | VVDVSQEDPEVQFNWYVDGVEVHNAKTKPRE | |
| | EQFNSTYRVVSVLTVLHQDWLNGKEYKCKVS | |
| | NKGLPSSIEKTISKAKGQPREPQVYTLPPSQEEM | |
| | TKNQVSLTCLVKGFYPSDIAVEWESNGQPENN | |
| | YKTTPPVLDSDGSFFLYSRLTVDKSRWQEGNV | |
| | FSCSVMHEALHNHYTQKSLSLSLGK | |
| LC | SYVLTQPPSVSVAPGQTARITCGGNNIGSKSVH | 13 |
| | WYQQPPGQAPVVVVYDDSDRPSGIPERFSGSN | |
| | SGNTATLTISRVEAGDEAVYYCQVWDSSSDHV | |
| | VFGGGTKLTVLGQPKAAPSVTLFPPSSEELQAN | |
| | KATLVCLISDFYPGAVTVAWKGDSSPVKAGVE | |
| | TTTPSKQSNNKYAASSYLSLTPEQWKSHRSYSC | |
| | QVTHEGSTVEKTVAPTECS | |

TABLE 4
Sequences of a CD3 binding arm

| | | SEQ |
|---|---|---|
| Region | Sequence | ID NO: |
| HCDR1 | TYAMN | 14 |
| HCDR2 | RIRSKYNNYATYYAASVKG | 15 |
| HCDR3 | HGNFGNSYVSWFAY | 16 |
| LCDR1 | RSSTGAVTTSNYAN | 17 |
| LCDR2 | GTNKRAP | 18 |
| LCDR3 | ALWYSNLWV | 19 |
| VH | EVQLVESGGGLVQPGGSLRLSCAASGFTENT | 20 |
| | YAMNWVRQAPGKGLEWVARIRSKYNNYAT | |
| | YYAASVKGRFTISRDDSKNSLYLQMNSLKTE | |
| | DTAVYYCARHGNFGNSYVSWFAYWGQGTL | |
| | VTVSS | |
| VL | QTVVTQEPSLTVSPGGTVTLTCRSSTGAVTT | 21 |
| | SNYANWVQQKPGQAPRGLIGGTNKRAPGTP | |
| | ARFSGSLLGGKAALTLSGVQPEDEAEYYCAL | |
| | WYSNLWVFGGGTKLTVLGQP | |
| HC | EVQLVESGGGLVQPGGSLRLSCAASGFTFNT | 22 |
| | YAMNWVRQAPGKGLEWVARIRSKYNNYAT | |
| | YYAASVKGRFTISRDDSKNSLYLQMNSLKTE | |
| | DTAVYYCARHGNFGNSYVSWFAYWGQGTL | |
| | VTVSSASTKGPSVFPLAPCSRSTSESTAALGC | |
| | LVKDYFPEPVTVSWNSGALTSGVHTFPAVL | |
| | QSSGLYSLSSVVTVPSSSLGTKTYTCNVDHK | |
| | PSNTKVDKRVESKYGPPCPPCPAPEAAGGPS | |
| | VFLFPPKPKDTLMISRTPEVTCVVVDVSQED | |
| | PEVQFNWYVDGVEVHNAKTKPREEQFNSTY | |
| | RVVSVLTVLHQDWLNGKEYKCKVSNKGLPS | |
| | SIEKTISKAKGQPREPQVYTLPPSQEEMTKNQ | |
| | VSLTCLVKGFYPSDIAVEWESNGQPENNYKT | |
| | TPPVLDSDGSFLLYSKLTVDKSRWQEGNVFS | |
| | CSVMHEALHNHYTQKSLSLSLGK | |
| LC | QTVVTQEPSLTVSPGGTVTLTCRSSTGAVTT | 23 |
| | SNYANWVQQKPGQAPRGLIGGTNKRAPGTP | |
| | ARFSGSLLGGKAALTLSGVQPEDEAEYYCAL | |
| | WYSNLWVFGGGTKLTVLGQPKAAPSVTLFP | |
| | PSSEELQANKATLVCLISDFYPGAVTVAWKA | |
| | DSSPVKAGVETTTPSKQSNNKYAASSYLSLT | |
| | PEQWKSHRSYSCQVTHEGSTVEKTVAPTECS | |

In some embodiments, the BCMA×CD3 bispecific antibody is CC-93269, BI 836909, JNJ-64007957 (teclistamab), or PF-06863135. In preferred embodiments, the BCMA×CD3 bispecific antibody is teclistamab (also referred to herein as Tec), which has the sequences described in Tables 3 and 4.

Teclistamab (also known as TECVAYLI®) is the first BCMA-directed bispecific antibody approved for the treatment of patients with relapsed or refractory multiple myeloma (RRMM). Teclistamab is a bispecific B-cell maturation antigen (BCMA)-directed CD3 T-cell engager indicated as a monotherapy for the treatment of adult patients with relapsed or refractory multiple myeloma who have received at least three or four prior lines of therapy, including a proteasome inhibitor, an immunomodulatory agent and an anti-CD38 monoclonal antibody. See, e.g., Usmani S Z, et al. Lancet 2021; 398:665-674, and Moreau P, et al. New Engl J Med 2022: 387: 495-505, which is incorporated by reference herein.

Teclistamab and its methods of use are described, for example, in WO 2017/031104, WO 2019/220369 and WO 2021/228783, which are incorporated by reference herein. According to particular embodiments, the BCMA×CD3 bispecific antibody has an amino acid sequence with at least 80%, at least 85%, at least 90%, at least 95%, at least 98%, or at least 99% sequence identity to the amino acid sequence of teclistamab.

Tables 5 and 6 provide sequences of an exemplary embodiment of a GPRC5D×CD3 bispecific antibody, according to the Kabat numbering system.

TABLE 5
Sequences of GPRC5D binding arm

| | | SEQ |
|---|---|---|
| | | ID |
| Region | Sequence | NO: |
| HCDR1 | GYTMN | 24 |
| HCDR2 | LINPYNSDTNYAQKLQG | 25 |
| HCDR3 | VALRVALDY | 26 |
| LCDR1 | KASQNVATHVG | 27 |
| LCDR2 | SASYRYS | 28 |
| LCDR3 | QQYNRYPYT | 29 |
| VH | QVQLVQSGAEVKKPGASVKVSCKASGYSF | 30 |
| | TGYTMNWVRQAPGQGLEWMGLINPYNSD | |
| | TNYAQKLQGRVTMTTDTSTSTAYMELRSL | |
| | RSDDTAVYYCARVALRVALDYWGQGTLV | |
| | TVSS | |
| VL | DIQMTQSPSSLSASVGDRVTITCKASQNVA | 31 |
| | THVGWYQQKPGKAPKRLIYSASYRYSGVP | |
| | SRFSGSGSGTEFTLTISNLQPEDFATYYCQQ | |
| | YNRYPYTFGQGTKLEIK | |
| HC | QVQLVQSGAEVKKPGASVKVSCKASGYSF | 32 |
| | TGYTMNWVRQAPGQGLEWMGLINPYNSD | |
| | TNYAQKLQGRVTMTTDTSTSTAYMELRSL | |
| | RSDDTAVYYCARVALRVALDYWGQGTLV | |
| | TVSSASTKGPSVFPLAPCSRSTSESTAALGC | |
| | LVKDYFPEPVTVSWNSGALTSGVHTFPAV | |
| | LQSSGLYSLSSVVTVPSSSLGTKTYTCNVD | |
| | HKPSNTKVDKRVESKYGPPCPPCPAPEAAG | |
| | GPSVFLFPPKPKDTLMISRTPEVTCVVVDV | |
| | SQEDPEVQFNWYVDGVEVHNAKTKPREE | |
| | QFNSTYRVVSVLTVLHQDWLNGKEYKCK | |
| | VSNKGLPSSIEKTISKAKGQPREPQVYTLPP | |
| | SQEEMTKNQVSLTCLVKGFYPSDIAVEWE | |
| | SNGQPENNYKTTPPVLDSDGSFFLYSRLTV | |
| | DKSRWQEGNVFSCSVMHEALHNHYTQKS | |
| | LSLSLGK | |
| LC | DIQMTQSPSSLSASVGDRVTITCKASQNVA | 33 |
| | THVGWYQQKPGKAPKRLIYSASYRYSGVP | |
| | SRFSGSGSGTEFTLTISNLQPEDFATYYCQQ | |
| | YNRYPYTFGQGTKLEIKRTVAAPSVFIFPPS | |
| | DEQLKSGTASVVCLLNNFYPREAKVQWK | |
| | VDNALQSGNSQESVTEQDSKDSTYSLSSTL | |
| | TLSKADYEKHKVYACEVTHQGLSSPVTKS | |
| | FNRGEC | |

TABLE 6
Sequences of CD3 binding arm

| | | SEQ |
|---|---|---|
| Region | Sequence | ID NO: |
| HCDR1 | TYAMN | 14 |
| HCDR2 | RIRSKYNNYATYYAASVKG | 15 |
| HCDR3 | HGNFGNSYVSWFAY | 16 |
| LCDR1 | RSSTGAVTTSNYAN | 17 |
| LCDR2 | GTNKRAP | 18 |
| LCDR3 | ALWYSNLWV | 19 |
| VH | EVQLVESGGGLVQPGGSLRLSCAASGFTENT | 20 |
| | YAMNWVRQAPGKGLEWVARIRSKYNNYAT | |
| | YYAASVKGRFTISRDDSKNSLYLQMNSLKTE | |
| | DTAVYYCARHGNFGNSYVSWFAYWGQGTL | |
| | VTVSS | |
| VL | QTVVTQEPSLTVSPGGTVTLTCRSSTGAVTT | 21 |
| | SNYANWVQQKPGQAPRGLIGGTNKRAPGTP | |
| | ARFSGSLLGGKAALTLSGVQPEDEAEYYCAL | |
| | WYSNLWVFGGGTKLTVLGQP | |
| HC | EVQLVESGGGLVQPGGSLRLSCAASGFTENT | 22 |
| | YAMNWVRQAPGKGLEWVARIRSKYNNYAT | |
| | YYAASVKGRFTISRDDSKNSLYLQMNSLKTE | |
| | DTAVYYCARHGNFGNSYVSWFAYWGQGTLV | |
| | TVSSASTKGPSVFPLAPCSRSTSESTAALGC | |
| | LVKDYFPEPVTVSWNSGALTSGVHTFPAVL | |
| | QSSGLYSLSSVVTVPSSSLGTKTYTCNVDHK | |
| | PSNTKVDKRVESKYGPPCPPCPAPEAAGGPS | |
| | VFLFPPKPKDTLMISRTPEVTCVVVDVSQED | |
| | PEVQFNWYVDGVEVHNAKTKPREEQFNSTY | |
| | RVVSVLTVLHQDWLNGKEYKCKVSNKGLPSS | |
| | IEKTISKAKGQPREPQVYTLPPSQEEMTKNQ | |
| | VSLTCLVKGFYPSDIAVEWESNGQPENNYKT | |
| | TPPVLDSDGSFLLYSKLTVDKSRWQEGNVFS | |
| | CSVMHEALHNHYTQKSLSLSLGK | |
| LC | QTVVTQEPSLTVSPGGTVTLTCRSSTGAVTT | 23 |
| | SNYANWVQQKPGQAPRGLIGGTNKRAPGTP | |
| | ARFSGSLLGGKAALTLSGVQPEDEAEYYCAL | |
| | WYSNLWVFGGGTKLTVLGQPKAAPSVTLFP | |
| | PSSEELQANKATLVCLISDFYPGAVTVAWKA | |
| | DSSPVKAGVETTTPSKQSNNKYAASSYLSLT | |
| | PEQWKSHRSYSCQVTHEGSTVEKTVAPTECS | |

In some embodiments, the GPRC5D×CD3 bispecific antibody is JNJ-64407564 or Talquetamab (also referred to herein as Tal), which has the sequences described in Tables 5 and 6.

Talquetamab is a GPRC5D-directed bispecific antibody in development for treatment of patients with relapsed or refractory multiple myeloma. See. e.g., Chari A, et al. Blood 2022: 140 (suppl 1): 384-387, which is incorporated by reference herein. Talquetamab is a bispecific GPRC5D-directed CD3 T cell engager in development as a monotherapy for the treatment of adult patients with relapsed or refractory multiple myeloma who have received at least three or four prior therapies, including a proteasome inhibitor, an immunomodulatory agent and an anti-CD38 monoclonal antibody.

Talquetamab and its methods of use are described, for example, in WO 2018/017786 and WO 2022/058445, which are incorporated by reference herein. According to particular embodiments, the GPRC5D×CD3 bispecific antibody has an amino acid sequence with at least 80%, at least 85%, at least 90%, at least 95%, at least 98%, or at least 99% sequence identity to the amino acid sequence of talquetamab.

Additional embodiments of BCMA×CD3 and GPRC5D×CD3 bispecific antibodies that may be used in combination regimens of the present invention are described below.

In certain embodiments, the BCMA×CD3 bispecific antibody comprises a BCMA binding domain comprising the HCDR1 of SEQ ID NO: 4, the HCDR2 of SEQ ID NO: 5, the HCDR3 of SEQ ID NO: 6, the LCDR1 of SEQ ID NO: 7, the LCDR2 of SEQ ID NO: 8 and the LCDR3 of SEQ ID NO: 9, and a CD3 binding domain comprising the HCDR1 of SEQ ID NO: 14, the HCDR2 of SEQ ID NO: 15, the HCDR3 of SEQ ID NO: 16, the LCDR1 of SEQ ID NO: 17, the LCDR2 of SEQ ID NO: 18 and the LCDR3 of SEQ ID NO: 19.

In certain embodiments, the BCMA binding domain comprises a heavy chain variable region (VH) having the amino acid sequence of SEQ ID NO: 10 and a light chain variable region (VL) having the amino acid sequence of SEQ ID NO: 11, and the CD3 binding domain comprises a heavy chain variable region (VH) having the amino acid sequence of SEQ ID NO: 20 and a light chain variable region (VL) having the amino acid sequence of SEQ ID NO: 21.

In certain embodiments, the BCMA×CD3 bispecific antibody is an IgG1, an IgG2, an IgG3 or an IgG4 isotype.

In certain embodiments, the BCMA×CD3 bispecific antibody is an IgG4 isotype.

In certain embodiments, the BCMA×CD3 bispecific antibody comprises one or more substitutions in its Fc region.

In certain embodiments, the BCMA×CD3 bispecific antibody is an IgG4 isotype and comprises S228P, F234A and L235A substitutions in its Fc region.

In certain embodiments, the BCMA×CD3 bispecific antibody is an IgG4 isotype and comprises S228P, F234A, L235A F405L and R409K substitutions in its Fc region.

In certain embodiments, the Fc region of the BCMA-specific IgG4 antibody from which the BCMA-binding arm is derived comprises S228P, L234A and L235A substitutions in its Fc region.

In certain embodiments, the Fc region of the CD3-specific IgG4 antibody from which the CD3-binding arm is derived comprises S228P, L234A, L235A, F405L, and R409K substitutions in its Fc region. In certain embodiments, the BCMA×CD3 bispecific antibody comprises a first heavy chain (HC1) having the amino acid sequence of SEQ ID NO: 12, a first light chain (LC1) having the amino acid sequence of SEQ ID NO: 13, a second heavy chain (HC2) having the amino acid sequence of SEQ ID NO: 22 and a second light chain (LC2) having the amino acid sequence of SEQ ID NO: 23.

In certain embodiments, the BCMA×CD3 bispecific antibody comprises a first heavy chain (HC1) having at least 90% identity to the amino acid sequence of SEQ ID NO: 12, a first light chain (LC1) having at least 90% identity to the amino acid sequence of SEQ ID NO: 13, a second heavy chain (HC2) having at least 90% identity to the amino acid sequence of SEQ ID NO: 22 and a second light chain (LC2) having at least 90% identity to the amino acid sequence of SEQ ID NO: 23.

In certain embodiments, the BCMA×CD3 bispecific antibody comprises a first heavy chain (HC1) having at least 95% identity to the amino acid sequence of SEQ ID NO: 12, a first light chain (LC1) having at least 95% identity to the amino acid sequence of SEQ ID NO: 13, a second heavy chain (HC2) having at least 95% identity to the amino acid sequence of SEQ ID NO: 22 and a second light chain (LC2) having at least 95% identity to the amino acid sequence of SEQ ID NO: 23.

In certain embodiments, the BCMA×CD3 bispecific antibody comprises a first heavy chain (HC1) having at least 98% identity to the amino acid sequence of SEQ ID NO: 12, a first light chain (LC1) having at least 98% identity to the amino acid sequence of SEQ ID NO: 13, a second heavy chain (HC2) having at least 98% identity to the amino acid sequence of SEQ ID NO: 22 and a second light chain (LC2) having at least 98% identity to the amino acid sequence of SEQ ID NO: 23.

In certain embodiments, the BCMA×CD3 bispecific antibody is teclistamab.

In certain embodiments, the GPRC5D×CD3 bispecific antibody comprises a GPRC5D binding domain comprising the HCDR1 of SEQ ID NO: 24, the HCDR2 of SEQ ID NO: 25, the HCDR3 of SEQ ID NO: 26, the LCDR1 of SEQ ID NO: 27, the LCDR2 of SEQ ID NO: 28 and the LCDR3 of SEQ ID NO: 29, and a CD3 binding domain comprising the HCDR1 of SEQ ID NO: 14, the HCDR2 of SEQ ID NO: 15, the HCDR3 of SEQ ID NO: 16, the LCDR1 of SEQ ID NO: 17, the LCDR2 of SEQ ID NO: 18 and the LCDR3 of SEQ ID NO: 19.

In certain embodiments, the GPRC5D binding domain comprises a heavy chain variable region (VH) having the amino acid sequence of SEQ ID NO: 30 and a light chain variable region (VL) having the amino acid sequence of SEQ ID NO: 31, and the CD3 binding domain comprises a heavy chain variable region (VH) having the amino acid sequence of SEQ ID NO: 20 and a light chain variable region (VL) having the amino acid sequence of SEQ ID NO: 21.

In certain embodiments, the GPRC5D×CD3 bispecific antibody is an IgG1, an IgG2, an IgG3 or an IgG4 isotype.

In certain embodiments, the GPRC5D×CD3 bispecific antibody is an IgG4 isotype.

In certain embodiments, the GPRC5D×CD3 bispecific antibody comprises one or more substitutions in its Fc region.

In certain embodiments, the GPRC5D×CD3 bispecific antibody is an IgG4 isotype and comprises S228P. F234A and L235A substitutions in its Fc region.

In certain embodiments, the GPRC5D×CD3 bispecific antibody is an IgG4 isotype and comprises S228P, F234A, L235A F405L and R409K substitutions in its Fc region.

In certain embodiments, the Fc region of the GPRC5D-specific IgG4 antibody from which the GPRC5D-binding arm is derived comprises S228P. L234A and L235A substitutions in its Fc region.

In certain embodiments, the Fc region of the CD3-specific IgG4 antibody from which the CD3-binding arm is derived comprises S228P, L234A, L235A, F405L, and R409K substitutions in its Fc region.

In certain embodiments, the GPRC5D×CD3 bispecific antibody comprises a first heavy chain (HC1) having the amino acid sequence of SEQ ID NO: 32, a first light chain (LC1) having the amino acid sequence of SEQ ID NO: 33, a second heavy chain (HC2) having the amino acid sequence of SEQ ID NO: 22 and a second light chain (LC2) having the amino acid sequence of SEQ ID NO: 23.

In certain embodiments, the GPRC5D×CD3 bispecific antibody comprises a first heavy chain (HC1) having at least 90% identity to the amino acid sequence of SEQ ID NO: 32, a first light chain (LC1) having at least 90% identity to the amino acid sequence of SEQ ID NO: 33, a second heavy chain (HC2) having at least 90% identity to the amino acid sequence of SEQ ID NO: 22 and a second light chain (LC2) having at least 90% identity to the amino acid sequence of SEQ ID NO: 23.

In certain embodiments, the GPRC5D×CD3 bispecific antibody comprises a first heavy chain (HC1) having at least 95% identity to the amino acid sequence of SEQ ID NO: 32, a first light chain (LC1) having at least 95% identity to the amino acid sequence of SEQ ID NO: 33, a second heavy chain (HC2) having at least 95% identity to the amino acid sequence of SEQ ID NO: 22 and a second light chain (LC2) having at least 95% identity to the amino acid sequence of SEQ ID NO: 23.

In certain embodiments, the GPRC5D×CD3 bispecific antibody comprises a first heavy chain (HC1) having at least 98% identity to the amino acid sequence of SEQ ID NO: 32, a first light chain (LC1) having at least 98% identity to the amino acid sequence of SEQ ID NO: 33, a second heavy chain (HC2) having at least 98% identity to the amino acid sequence of SEQ ID NO: 22 and a second light chain (LC2) having at least 98% identity to the amino acid sequence of SEQ ID NO: 23.

In certain embodiments, the GPRC5D×CD3 bispecific antibody is talquetamab.

Patient Populations with Multiple Myeloma

The BCMA×CD3 and GPRC5D×CD3 bispecific antibodies disclosed herein are for use in treating multiple myeloma in a subject, for example a human subject, wherein the subject is relapsed or refractory to treatment with one or more prior anti-cancer treatments. Relapsed disease means a cancer has come back. Refractory disease means a cancer has not improved with treatment or has stopped responding to treatment.

In certain embodiments, the subject has relapsed or refractory multiple myeloma and received 1 to 4 prior lines of therapy including an anti-CD38 antibody and lenalidomide.

The inventors have further discovered that combination regimens described herein demonstrate high efficacy in patients with extramedullary disease (EMD). In certain embodiments, a patient has one or more focus of EMD meeting the following criterion: any extramedullary plasmacytoma not contiguous with a bone lesion ≥2 cm (at its greatest dimension) diameter on PET-CT and not previously radiated.

In some embodiments, the subject is relapsed or refractory to treatment with a therapeutic used to treat multiple myeloma or other hematological malignancies.

In certain embodiments, the subject has had been treated with from 1 to 11 prior lines of therapy, or from 1 to 10 prior lines of therapy.

In certain embodiments, the subject has previously received an autologous stem cell transplant (ASCT).

In certain embodiments, the subject has received at least three prior lines of therapy.

In certain embodiments, the subject has received at least four prior lines of therapy.

In certain embodiments, the subject has received at least five prior lines of therapy (penta-drug exposed).

In certain embodiments, the subject has received at least three prior lines of therapy, including a proteasome inhibitor, an immunomodulatory agent and an anti-CD38 monoclonal antibody.

In certain embodiments, the subject has received at least four prior lines of therapy, including a proteasome inhibitor, an immunomodulatory agent and an anti-CD38 monoclonal antibody.

In particular embodiments, the patients are relapsed or refractory or intolerant to the last line of therapy (LOT), were exposed to a proteasome inhibitor, immunomodulatory drug, and anti-CD38 therapy; and had measurable disease.

In some embodiments, the subject has received three anti-cancer therapies prior to administration of the BCMA×CD3 bispecific antibody and GPRC5D×CD3 bispecific antibody.

In one embodiment, the three prior anti-cancer therapies are a proteasome inhibitor (PI), an immunomodulatory drug (IMiD), and an anti-CD38 antibody. In certain such embodiments, the proteasome inhibitor is bortezomib, carfilzomib or ixazomib, the immunomodulatory drug (IMiD) is lenalidomide, pomalidomide or thalidomide and the anti-CD38 antibody is daratumumab or isatuximab.

In one embodiment, the proteasome inhibitor is bortezomib, the immunomodulatory drug (IMiD) is lenalidomide and the anti-CD38 antibody is daratumumab. In one embodiment, the proteasome inhibitor is bortezomib, the immunomodulatory drug (IMiD) is lenalidomide and the anti-CD38 antibody is isatuximab. In one embodiment, the proteasome inhibitor is bortezomib, the immunomodulatory drug (IMiD) is pomalidomide and the anti-CD38 antibody is daratumumab. In one embodiment, the proteasome inhibitor is bortezomib, the immunomodulatory drug (IMiD) is pomalidomide and the anti-CD38 antibody is isatuximab. In one embodiment, the proteasome inhibitor is bortezomib, the immunomodulatory drug (IMiD) is thalidomide and the anti-CD38 antibody is daratumumab. In one embodiment, the proteasome inhibitor is bortezomib, the immunomodulatory drug (IMiD) is thalidomide and the anti-CD38 antibody is isatuximab.

In one embodiment, the proteasome inhibitor is carfilzomib, the immunomodulatory drug (IMiD) is lenalidomide and the anti-CD38 antibody is daratumumab. In one embodiment, the proteasome inhibitor is carfilzomib, the immunomodulatory drug (IMiD) is lenalidomide and the anti-CD38 antibody is isatuximab. In one embodiment, the proteasome inhibitor is carfilzomib, the immunomodulatory drug (IMiD) is pomalidomide and the anti-CD38 antibody is daratumumab. In one embodiment, the proteasome inhibitor is carfilzomib, the immunomodulatory drug (IMiD) is pomalidomide and the anti-CD38 antibody is isatuximab. In one embodiment, the proteasome inhibitor is carfilzomib, the immunomodulatory drug (IMiD) is thalidomide and the anti-CD38 antibody is daratumumab. In one embodiment, the proteasome inhibitor is carfilzomib, the immunomodulatory drug (IMiD) is thalidomide and the anti-CD38 antibody is isatuximab.

In one embodiment, the proteasome inhibitor is ixazomib, the immunomodulatory drug (IMiD) is lenalidomide and the anti-CD38 antibody is daratumumab. In one embodiment, the proteasome inhibitor is ixazomib, the immunomodulatory drug (IMiD) is lenalidomide and the anti-CD38 antibody is isatuximab. In one embodiment, the proteasome inhibitor is ixazomib, the immunomodulatory drug (IMiD) is pomalidomide and the anti-CD38 antibody is daratumumab. In one embodiment, the proteasome inhibitor is ixazomib, the immunomodulatory drug (IMiD) is pomalidomide and the anti-CD38 antibody is isatuximab. In one embodiment, the proteasome inhibitor is ixazomib, the immunomodulatory drug (IMiD) is thalidomide and the anti-CD38 antibody is daratumumab. In one embodiment, the proteasome inhibitor is ixazomib, the immunomodulatory drug (IMiD) is thalidomide and the anti-CD38 antibody is isatuximab.

In some embodiments, the subject is refractory or relapsed to treatment with one or more treatments or therapies, such as THALOMID (thalidomide), REVLIMID® (lenalidomide), POMALYST® (pomalidomide), VELCADE® (bortezomib), NINLARO (ixazomib), KYPROLIS® (carfilzomib), FARADYK (panobinostat), AREDIA® (pamidronate), ZOMETA® (zoledronic acid), DARZALEX® (daratumumab), elotozumab or melphalan, Xpovio® (Selinexor), Venclexta® (Venetoclax), GSK 916, CAR-T therapies, or other BCMA-directed therapies.

Various qualitative and/or quantitative methods can be used to determine relapse or refractory nature of the disease. Symptoms that can be associated are for example a decline or plateau of the well-being of the patient or re-establishment or worsening of various symptoms associated with solid tumors, and/or the spread of cancerous cells in the body from one location to other organs, tissues or cells.

In some embodiments, the multiple myeloma is relapsed or refractory to treatment with an anti-CD38 antibody, selinexor, venetoclax, lenalinomide, bortezomib, pomalidomide, carfilzomib, elotozumab, ixazomib, melphalan or thalidomide, or any combination thereof.

In one embodiment, the anti-CD38 antibody is daratumumab.

In another embodiment, the anti-CD38 antibody is isatuximab.

In some embodiments, the multiple myeloma is a high-risk multiple myeloma. Subjects with high-risk multiple myeloma are known to relapse early and have poor prognosis and outcome. Subjects can be classified as having high-risk multiple myeloma is they have one or more of the following cytogenetic abnormalities: t(4:14)(p16;q32), t(14;16)(q32;q23), del17p, 1qAmp, t(4;14)(p16;q32) and t(14;16)(q32;q23), t(4.14)(p16;q32) and del17p, t(14;16)(q32;q23) and del17p, or t(4:14)(p16;q32), t(14:16)(q32;q23) and del17p. In some embodiments, the subject having the high-risk multiple myeloma has one or more chromosomal abnormalities comprising: t(4:14)(p16;q32), t(14:16)(q32;q23), del17p, 1qAmp, t(4;14)(p16;q32) and t(14;16)(q32;q23), t(4;14)(p16;q32) and del17p, t(14;16)(q32;q23) and del17p; or t(4;14)(p16;q32), t(14;16)(q32;q23) and del17p, or any combination thereof.

The cytogenetic abnormalities can be detected for example by fluorescent in situ hybridization (FISH). In chromosomal translocations, an oncogene is translocated to the IgH region on chromosome 14q32, resulting in dysregulation of these genes, t(4:14)(p16;q32) involves translocation of fibroblast growth factor receptor 3 (FGFR3) and multiple myeloma SET domain containing protein (MMSET) (also called WHSC1/NSD2), and t(14;16)(q32;q23) involves translocation of the MAF transcription factor C-MAF. Deletion of 17p (del17p) involves loss of the p53 gene locus.

Chromosomal rearrangements can be identified using well known methods, for example fluorescent in situ hybridization, karyotyping, pulsed field gel electrophoresis, or sequencing.

### Combination Dosing Regimens

The inventors have found that subjects in need of treatment for multiple myeloma that are relapsed or refractory to treatment with prior anti-cancer treatment(s) may be particularly amenable to treatment with a combination dosing regimen comprising both a BCMA×CD3 bispecific antibody (e.g., teclistamab or “Tec”) and a GPRC5D×CD3 bispecific antibody (e.g., talquetamab or “Tal”).

As used herein, “weight-based” refers to administration of a dose amount that is based on the subject's specific body weight; for example, 3 mg/kg refers to a dose of 3 milligrams of antibody per kilogram of the subject's body weight. Unless otherwise specified herein, when a dose is described in a unit of “mg/kg” or “μg/kg,” weight-based dosing is being employed.

Unless otherwise specified herein, a BCMA×CD3 or GPRC5D×CD3 bispecific antibody, such as teclistamab or talquetamab, is administered on a dosing schedule based on sequential 28-day cycles, for example, Cycle 1 starts on Day 1 of Cycle 1 and ends on Day 28 of Cycle 1, and then Day 1 of Cycle 2 starts the day after Day 28 of Cycle 1 and ends on Day 28 of Cycle 2, and then Day 1 of Cycle 3 starts the day after Day 28 of Cycle 2 and ends on Day 28 of Cycle 3, and so on. In certain embodiments, one or more step-up doses are administered prior to the first treatment cycle, i.e., prior to Cycle 1 Day 1. In other embodiments, one or more step-up doses are administered during Cycle 1 (e.g., the first step-up dose may be administered on Cycle 1 Day 1).

A treatment cycle, as used herein, refers to a 28-day treatment cycle. As used herein with respect to treatment cycles, “C1” refers to Cycle 1, “C2” refers to Cycle 2, “C3” refers to Cycle 3, and so on. Multiple cycles may also be described, e.g., “C3-6” refers to Cycles 3-6 (Cycles 3, 4, 5 and 6). A cycle number with a “+” symbol refers to that cycle and all subsequent cycles, e.g., “C5+” refers to from Cycle 5 and all subsequent cycles (i.e., C5, C6, C7, C8, C9, and so on).

As used herein “Q4W” means once every four weeks, “Q2W” (also referred to as “bi-weekly” or “biweekly”) means once every two weeks, and “QW” (also referred to as “weekly”) means once weekly. Q4W may be referred to herein as “monthly” but technically refers to once every 4 weeks or once every 28 days (e.g., in 28-day cycles, a first treatment dose occurs on Day 1 of Cycle 1, a second treatment dose occurs on Day 1 of Cycle 2, etc.). Administration of a treatment dose once weekly (QW) is also referred to herein as a weekly dosing schedule; for example, a 28-day treatment cycle may have a weekly dosing schedule that comprises four doses one week apart from each other (e.g., on Days 1, 8, 15 and 22), or three doses one week apart from each other (e.g., on Days 8, 15 and 22), or two doses one week apart from each other (e.g., on Days 8 and 15). Administration of a treatment dose once every two weeks (Q2W) is also referred to herein as a bi-weekly dosing schedule. Administration of a treatment dose once every four weeks (Q4W) is also referred to herein as a monthly dosing schedule. Dosing regimens may be described herein in terms of the dose amount and frequency; for example, “C1: 0.4 mg/kg QW” refers to administration of 0.4 mg/kg once per week in Cycle 1 of a therapeutically effective regimen, “C3-6: 0.8 mg/kg Q2W” refers to administration of 0.8 mg/kg once every two weeks from Cycle 3 through Cycle 6, “C7+: 0.8 mg/kg Q4W” refers to administration of 0.8 mg/kg once every four weeks starting in Cycle 7, etc.

As used herein, a “BCMA×CD3 treatment cycle” refers to each treatment cycle in a therapeutically effective regimen in which at least one treatment dose of a BCMA×CD3 bispecific antibody is administered to the subject.

As used herein, a “GPRC5D×CD3 treatment cycle” refers to each treatment cycle in a therapeutically effective regimen in which at least one treatment dose of a GPRC5D×CD3 bispecific antibody is administered to the subject.

According to particular embodiments, a subject is subcutaneously administered (i) 3.0 mg/kg of Teclistamab every two weeks (Q2W), and (ii) 0.8 mg/kg of Talquetamab every two weeks (Q2W).

According to particular embodiments, the method comprises administering talquetamab in combination with teclistamab for the treatment of adult patients with relapsed or refractory multiple myeloma with extramedullary disease who have received a proteasome inhibitor, an immunomodulatory agent and an anti-CD38 monoclonal antibody.

According to embodiments of the present invention, methods of treating multiple myeloma are effective in eliciting a clinical response in a subject as determined by International Myeloma Working Group (IMWG) response criteria. According to particular embodiments, the methods of treatment are effective in eliciting a partial response, a very good partial response, a complete response or a stringent complete response, as determined by IMWG response criteria. As used herein, overall response rate (ORR) refers to the percentage of patients in a population that achieve a partial response (PR) or better, i.e., a partial response, very good partial response, complete response or stringent complete response. IMWG criteria for response to Multiple Myeloma treatment are provided in Table 7 below.

TABLE 7

| Response | Response Criteria |
|---|---|
| sCR = stringent | CR as defined below, plus |
| complete | Normal FLC ratio, and |
| response | Absence of clonal PCs by immunohistochemistry, |
| | immunofluorescenceª or 2- to 4-color flow cytometry |
| CR = complete | Negative immunofixation on the serum and urine, |
| response | and |
| | Disappearance of any soft tissue plasmacytomas, |
| | and |
| | <5% PCs in bone marrow |
| VGPR = very | Serum and urine M-component detectable by |
| good partial | immunofixation but not on electrophoresis, or |
| response | ≥90% reduction in serum M-protein plus urine M- |
| | protein <100 mg/24 hours |
| PR = partial | ≥50% reduction of serum M-protein and reduction |
| response | in 24-hour urinary M-protein by ≥90% or to <200 |
| | mg/24 hours |
| | If the serum and urine M-protein are not |
| | measurable, a decrease of ≥50% in the difference |
| | between involved and uninvolved FLC levels is |
| | required in place of the M-protein criteria |
| | If serum and urine M-protein are not measurable, |
| | and serum free light assay is also not measurable, |
| | ≥50% reduction in bone marrow PCs is required in |
| | place of M-protein, |
| | provided baseline bone marrow plasma cell |
| | percentage was ≥30% |
| | In addition to the above criteria, if present at |
| | baseline, a ≥50% reduction in the size of soft tissue |
| | plasmacytomas is also required. |

CR = complete response;
FLC = free light chain;
IMWG = International Myeloma Working Group;
M-protein = monoclonal paraprotein;
MR = minimal response;
PC = plasma cell;
PD = progressive disease;
PR = partial response;
sCR = stringent complete response;
SD = stable disease;
VGPR = very good partial response
^{a}Presence/absence of clonal cells is based upon the kappa/lambda ratio. An abnormal kappa/lambda ratio by immunohistochemistry or immunofluorescence requires a minimum of 100 plasma cells for analysis. An abnormal ratio reflecting presence of an abnormal clone is kappa/lambda of >4:1 or <1:2.
*Clarifications to IMWG criteria for coding CR and VGPR in subjects in whom the only measurable disease is by serum FLC levels: CR in such subjects indicates a normal FLC ratio of 0.26 to 1.65 in addition to CR criteria listed above. VGPR in such subjects requires a >90% decrease in the difference between involved and uninvolved FLC levels.

IMWG criteria for response to Multiple Myeloma treatment are also described, for example, in Durie et al., Kumar et al, and Rajkumar et al., which are incorporated by reference herein: Durie B G, Harousseau J L, Miguel J S, et al. International uniform response criteria for multiple myeloma. Leukemia. 2006; 20(9):1467-1473; Kumar S. Paiva B. Anderson K C, et al. International Myeloma Working Group consensus criteria for response and minimal residual disease assessment in multiple myeloma. Lancet Oncol. 2016;17(8):e328-346; Rajkumar S V, Harousseau J L, Durie B. et al. Consensus recommendations for the uniform reporting of clinical Trials: report of the International Myeloma Workshop Consensus Panel 1. Blood. 2011:117(18):4691-4695.

In certain embodiments, a combination dosing regimen comprising a BCMA×CD3 bispecific antibody (e.g., techstamab) and a GPRC5D×CD3 bispecific antibody (e.g., talquetamab) is safe and well-tolerated in patients. In certain embodiments, treatment of a subject with the combination dosing regimen does not result in additive toxicity; for example, administration of the combination dosing regimen to a patient population may result in incidence and severity of adverse events that are consistent with incidence and severity of adverse events observed in patient populations treated with either monotherapy regimen (teclistamab or talquetamab).

In certain embodiments, the overall response rate in subjects treated with a combination dosing regimen comprising a BCMA×CD3 bispecific antibody (e.g., teclistamab) and a GPRC5D×CD3 bispecific antibody (e.g., talquetamab) is at least about 55%, at least about 60%, at least about 65%, at least about 70%, at least about 75%, at least about 80%, at least about 85% or at least about 90% of treated subjects. In one embodiment, treatment with a combination dosing regimen comprising teclistamab and talquetamab achieves an overall response rate of about 92% ORR in patients with advanced relapsed/refractory multiple myeloma and an overall response rate of about 83% in patients with extramedullary disease (EMD), a high-risk population with unmet need. In certain embodiments, the population of patients have previously received a median of 4 lines of therapy (e.g., from 1 to 10, or from 1 to 11 prior lines of therapy). In certain embodiments, patients have previously received an autologous stem cell transplant (ASCT). In certain embodiments, the proportion of clinical responses (PR or better) in patients with EMD is higher in patients that receive a combination regimen comprising teclistamab and talquetamab compared to the proportion of clinical responses (PR or better) in patients with EMD that receive talquetamab monotherapy or teclistamab monotherapy. For instance, as described in Example 1 herein, in patients with EMD, responses with talquetamab monotherapy were 45.5% (0.4 mg/kg QW) and 40.0% (0.8 mg/kg Q2W) and 37.5% with teclistamab monotherapy in other clinical trials (see, e.g., Moreau P, Garfall A L, van de Donk N. et al. Teclistamab in relapsed or refractory multiple myeloma. N Engl J Med 2022; 387:495-505; and Chari A, Minnema M C, Berdeja J G, et al. Talquetamab, a T-cell-redirecting GPRC5D bispecific antibody for multiple myeloma. N Engl J Med 2022; 387:2232-44); whereas in RedirecTT-1 (teclistamab+talquetamab), 66.7% of patients with EMD had a response at dose level 5 (33.3% had a complete response or better) and the probability of remaining in response at 18 months was 81.8%.

In another embodiment, treatment with a combination dosing regimen comprising teclistamab and talquetamab achieves an overall response rate of about 96% ORR in patients with advanced relapsed/refractory multiple myeloma and an overall response rate of about 86% in patients with extramedullary disease (EMD), a high-risk population with unmet need. In certain embodiments, the population of patients have previously received a median of 4 lines of therapy (e.g., from 1 to 10, or from 1 to 11 prior lines of therapy). In certain embodiments, patients have previously received an autologous stem cell transplant (ASCT).

In certain embodiments, teclistamab may be administered subcutaneously according to the following weight-based dosing schedule shown in Table 8A, wherein mg/kg refers to mg of teclistamab per kg of the patient's body weight;

TABLE 8A

| Dosing | | | | | |
|---|---|---|---|---|---|
| Schedule | Day | | Dose | | |
| Step-up | Day | 1 | Step-up dose 1 | 0.06 | mg/kg |
| Dosing | Day | 4 ^{b} | Step-up dose 2 | 0.3 | mg/kg |
| Schedule | Day | 7 ^{c} | First treatment | ≥1.5 | mg/kg |

| | | | |
|---|---|---|---|
| | | dose | |
| Weekly | One week after | Subsequent | ≥1.5 mg/kg once |
| Dosing | first treatment | treatment doses | weekly |
| Schedule | dose and weekly | | |
| | thereafter | | |

^{b} Step-up dose 2 may be given between 2 to 4 days after step-up dose 1 and may be given up to 7 days after step-up dose 1 to allow for resolution of adverse reactions.
^{c} First treatment dose may be given between 2 to 4 days after step-up dose 2 and may be given up to 7 days after step-up dose 2 to allow for resolution of adverse reactions.

In certain embodiments, teclistamab may be administered subcutaneously according to the following weight-based dosing schedule shown in Table 8B, wherein mg/kg refers to mg of teclistamab per kg of the patient's body weight;

TABLE 8B

| | | |
|---|---|---|
| Dosing | | |
| Schedule | Day | Dose |

All Patients

| | | | | | |
|---|---|---|---|---|---|
| Step-up | Day | 1 | Step-up dose 1 | 0.06 | mg/kg |
| Dosing | Day | 4^{a} | Step-up dose 2 | 0.3 | mg/kg |
| Schedule | Day | 7^{b} | Step-up dose 3 | 1.5 | mg/kg |
| First | Day | 10^{c} | First treatment | 3.0 | mg/kg |

| | | | |
|---|---|---|---|
| treatment | | dose | |
| dose | | | |
| Biweekly | Once every | Subsequent | 3.0 mg/kg once |
| (every two | two weeks | treatment doses | every two weeks |
| weeks) | thereafter | | |
| Treatment | | | |
| Dose | | | |
| Schedule | | | |

^{a}Step-up dose 2 may be given between 2 to 4 days after step-up dose 1 and may be given up to 7 days after step-up dose 1 to allow for resolution of adverse reactions.
^{b}Step-up dose 3 may be given between 2 to 4 days after step-up dose 2 and may be given up to 7 days after step-up dose 1 to allow for resolution of adverse reactions.
^{c}First treatment dose may be given between 2 to 4 days after step-up dose 2 and may be given up to 7 days after step-up dose 2 to allow for resolution of adverse reactions.

In certain embodiments, teclistamab may be subcutaneously adminstered according to either of the following weight-based dosing schedule shown in Table 9 and 10, wherein mg/kg refers to mg of teclistamab per kg of the patient's body weight;

TABLE 9

| Phase | Day | | Talquetamab Doseª | |
|---|---|---|---|---|
| Step-up Phase | Day | 1 | 0.01 | mg/kg |
| | Day | 3^{b} | 0.06 | mg/kg |
| | Day | 5^{b} | 0.4 | mg/kg |

| | | | |
|---|---|---|---|
| Treatment Phase | Once a week thereafter^{c} | 0.4 | mg/kg |

^{a}Based on actual body weight.
bDose may be administered between 2 to 4 days after the previous dose and may be given up to 7 days after the previous dose to allow for resolution of adverse reactions.
^{c}Maintain a minimum of 6 days between weekly doses and a minimum of 12 days between biweekly (every 2 weeks) doses.

TABLE 10

| Phase | Day | | Talquetamab Doseª | |
|---|---|---|---|---|
| Step-up Phase | Day | 1 | 0.01 | mg/kg |
| | Day | 3^{b} | 0.06 | mg/kg |
| | Day | 5^{b} | 0.4 | mg/kg |
| | Day | 7^{b} | 0.8 | mg/kg |

| | | | |
|---|---|---|---|
| Treatment Phase | Once every 2 weeks | 0.8 | mg/kg |

thereafter^{c}

^{a}Based on actual body weight.
^{b}Dose may be administered between 2 to 4 days after the previous dose and may be given up to 7 days after the previous dose to allow for resolution of adverse reactions.
^{c}Maintain a minimum of 6 days between weekly doses and a minimum of 12 days between biweekly (every 2 weeks) doses.

Additional embodiments of combination regimens of the present invention are described below.

In certain embodiments, the method comprises subcutaneously administering to the subject one or more step-up doses of the BCMA×CD3 bispecific antibody prior to administering a treatment dose of the BCMA×CD3 bispecific antibody.

In certain embodiments, the method comprises subcutaneously administering to the subject a treatment dose of the BCMA×CD3 bispecific antibody weekly (QW).

In certain embodiments, the method comprises subcutaneously administering to the subject a treatment dose of the BCMA×CD3 bispecific antibody every two weeks (Q2W).

In certain embodiments, the method comprises subcutaneously administering the BCMA×CD3 bispecific antibody weekly (QW) at a treatment dose of from about 750 μg/kg to about 3000 μg/kg.

In certain embodiments, the method comprises subcutaneously administering the BCMA×CD3 bispecific antibody weekly (QW) at a treatment dose of about 750 μg/kg.

In certain embodiments, the method comprises subcutaneously administering the BCMA×CD3 bispecific antibody weekly (QW) at a treatment dose of about 1500 μg/kg.

In certain embodiments, the method comprises subcutaneously administering the BCMA×CD3 bispecific antibody weekly (QW) at a treatment dose of about 3000 μg/kg.

In certain embodiments, the method comprises subcutaneously administering the BCMA×CD3 bispecific antibody every two weeks (Q2W) at a treatment dose of from about 750 μg/kg to about 3000 μg/kg.

In certain embodiments, the method comprises subcutaneously administering the BCMA×CD3 bispecific antibody every two weeks (Q2W) at a treatment dose of from about 1500 μg/kg to about 3000 μg/kg.

In certain embodiments, the method comprises subcutaneously administering the BCMA×CD3 bispecific antibody every two weeks (Q2W) at a treatment dose of 750 μg/kg.

In certain embodiments, the method comprises subcutaneously administering the BCMA×CD3 bispecific antibody every two weeks (Q2W) at a treatment dose of about 1500 μg/kg.

In certain embodiments, the method comprises subcutaneously administering the BCMA×CD3 bispecific antibody every two weeks (Q2W) at a treatment dose of about 3000 μg/kg.

In certain embodiments, the method comprises subcutaneously administering 2 or 3 step-up doses of the BCMA×CD3 bispecific antibody prior to subcutaneously administering the treatment dose.

In certain embodiments, the method comprises subcutaneously administering step-up doses of 60 μg/kg and 300 μg/kg of the BCMA×CD3 bispecific antibody prior to subcutaneously administering a treatment dose.

In certain embodiments, the method comprises subcutaneously administering step-up doses of 60 μg/kg and 300 μg/kg and 1500 μg/kg of the BCMA×CD3 bispecific antibody prior to subcutaneously administering a treatment dose.

In certain embodiments, the method comprises subcutaneously administering step-up doses of the BCMA×CD3 bispecific antibody 2-4 days apart from each other.

In certain embodiments, prior to subcutaneously administering a treatment dose of the BCMA×CD3 bispecific antibody, the method comprises: subcutaneously administering a first step-up dose of 60 μg/kg of the BCMA×CD3 bispecific antibody, then 2-4 days later subcutaneously administering a second step-up dose of 300 μg/kg of the BCMA×CD3 bispecific antibody, then 2-4 days later subcutaneously administering a third step-up dose of 1500 μg/kg of the BCMA×CD3 bispecific antibody.

In certain embodiments, the method comprises subcutaneously administering to the subject one or more step-up doses of the GPRC5D×CD3 bispecific antibody prior to administering a treatment dose of the GPRC5D×CD3 bispecific antibody.

In certain embodiments, the method comprises subcutaneously administering to the subject a treatment dose of the GPRC5D×CD3 bispecific antibody weekly (QW).

In certain embodiments, the method comprises subcutaneously administering to the subject a treatment dose of the GPRC5D×CD3 bispecific antibody every two weeks (Q2W).

In certain embodiments, the method comprises subcutaneously administering the GPRC5D×CD3 bispecific antibody weekly (QW) at a treatment dose of from about 200 μg/kg to about 800 μg/kg.

In certain embodiments, the method comprises subcutaneously administering the GPRC5D×CD3 bispecific antibody weekly (QW) at a treatment dose of about 200 μg/kg.

In certain embodiments, the method comprises subcutaneously administering the GPRC5D×CD3 bispecific antibody weekly (QW) at a treatment dose of about 400 μg/kg.

In certain embodiments, the method comprises subcutaneously administering the GPRC5D×CD3 bispecific antibody weekly (QW) at a treatment dose of about 800 μg/kg.

In certain embodiments, the method comprises subcutaneously administering the GPRC5D×CD3 bispecific antibody every two weeks (Q2W) at a treatment dose of from about 200 μg/kg to about 800 μg/kg.

In certain embodiments, the method comprises subcutaneously administering the GPRC5D×CD3 bispecific antibody every two weeks (Q2W) at a treatment dose of about 200 μg/kg.

In certain embodiments, the method comprises subcutaneously administering the GPRC5D×CD3 bispecific antibody every two weeks (Q2W) at a treatment dose of about 400 μg/kg.

In certain embodiments, the method comprises subcutaneously administering the GPRC5D×CD3 bispecific antibody every two weeks (Q2W) at a treatment dose of about 800 μg/kg.

In certain embodiments, the method comprises subcutaneously administering 2 or 3 step-up doses of the GPRC5D×CD3 bispecific antibody prior to subcutaneously administering the treatment dose.

In certain embodiments, the method comprises subcutaneously administering step-up doses of 10 μg/kg and 60 μg/kg of the GPRC5D×CD3 bispecific antibody prior to subcutaneously administering a treatment dose.

In certain embodiments, the method comprises subcutaneously administering step-up doses of 10 μg/kg and 60 μg/kg and 400 μg/kg of the GPRC5D×CD3 bispecific antibody prior to subcutaneously administering a treatment dose.

In certain embodiments, the method comprises subcutaneously administering step-up doses of the GPRC5D×CD3 bispecific antibody 2-4 days apart from each other.

In certain embodiments, prior to subcutaneously administering a treatment dose of the GPRC5D×CD3 bispecific antibody, the method comprises: subcutaneously administering a first step-up dose of 10 μg/kg of the GPRC5D×CD3 bispecific antibody, then 2-4 days later subcutaneously administering a second step-up dose of 60 μg/kg of the GPRC5D×CD3 bispecific antibody, then 2-4 days later subcutaneously administering a third step-up dose of 400 μg/kg of the GPRC5D×CD3 bispecific antibody.

In certain embodiments, the method comprises subcutaneously administering step-up doses of 10 μg/kg and 60 μg/kg and 300 μg/kg of the GPRC5D×CD3 bispecific antibody prior to subcutaneously administering a treatment dose.

In certain embodiments, the method comprises subcutaneously administering step-up doses of the GPRC5D×CD3 bispecific antibody 2-4 days apart from each other.

In certain embodiments, prior to subcutaneously administering a treatment dose of the GPRC5D×CD3 bispecific antibody, the method comprises: subcutaneously administering a first step-up dose of 10 μg/kg of the GPRC5D×CD3 bispecific antibody, then 2-4 days later subcutaneously administering a second step-up dose of 60 μg/kg of the GPRC5D×CD3 bispecific antibody, then 2-4 days later subcutaneously administering a third step-up dose of 300 μg/kg of the GPRC5D×CD3 bispecific antibody.

In certain embodiments, the method comprises subcutaneously administering the BCMA×CD3 bispecific antibody every two weeks (Q2W) at a treatment dose of 3000 μg/kg and subcutaneously administering the GPRC5D×CD3 bispecific antibody every two weeks (Q2W) at a treatment dose of 800 μg/kg.

In certain embodiments, the method comprises subcutaneously administering the BCMA×CD3 bispecific antibody every two weeks (Q2W) at a treatment dose of 1500 μg/kg and subcutaneously administering the GPRC5D×CD3 bispecific antibody every two weeks (Q2W) at a treatment dose of 800 μg/kg.

In certain embodiments, the method comprises subcutaneously administering the BCMA×CD3 bispecific antibody weekly (QW) at a treatment dose of 1500 μg/kg and subcutaneously administering the GPRC5D×CD3 bispecific antibody weekly (QW) at a treatment dose of 800 μg/kg.

In certain embodiments, the method comprises subcutaneously administering the BCMA×CD3 bispecific antibody weekly (QW) at a treatment dose of 1500 μg/kg and subcutaneously administering the GPRC5D×CD3 bispecific antibody weekly (QW) at a treatment dose of 400 μg/kg.

In certain embodiments, the method comprises subcutaneously administering the BCMA×CD3 bispecific antibody weekly (QW) at a treatment dose of 1500 μg/kg and subcutaneously administering the GPRC5D×CD3 bispecific antibody weekly (QW) at a treatment dose of 200 μg/kg.

In certain embodiments, the method comprises subcutaneously administering the BCMA×CD3 bispecific antibody weekly (QW) at a treatment dose of 750 μg/kg and subcutaneously administering the GPRC5D×CD3 bispecific antibody weekly (QW) at a treatment dose of 200 μg/kg.

In certain embodiments, the method comprises subcutaneously administering one or more step-up doses of the BCMA×CD3 bispecific antibody prior to subcutaneously administering a treatment dose; and subcutaneously administering one or more step-up doses of the GPRC5D×CD3 bispecific antibody prior to subcutaneously administering a treatment dose.

In certain embodiments, the method comprises subcutaneously administering 2 or 3 step-up doses of the BCMA×CD3 bispecific antibody prior to subcutaneously administering a treatment dose; and subcutaneously administering 2 or 3 step-up doses of the GPRC5D×CD3 bispecific antibody prior to subcutaneously administering a treatment dose.

In certain embodiments, the method comprises subcutaneously administering step-up doses of 60 μg/kg and 300 μg/kg of the BCMA×CD3 bispecific antibody prior to subcutaneously administering a treatment dose; and subcutaneously administering step-up doses of 10 μg/kg and 60 μg/kg of the GPRC5D×CD3 bispecific antibody prior to subcutaneously administering a treatment dose.

In certain embodiments, the method comprises subcutaneously administering step-up doses of 60 μg/kg and 300 μg/kg and 1500 μg/kg of the BCMA×CD3 bispecific antibody prior to subcutaneously administering a treatment dose; and subcutaneously administering step-up doses of 10 μg/kg and 60 μg/kg and 300 μg/kg of the GPRC5D×CD3 bispecific antibody prior to subcutaneously administering a treatment dose.

In certain embodiments, the method comprises subcutaneously administering step-up doses of 60 μg/kg and 300 μg/kg and 1500 μg/kg of the BCMA×CD3 bispecific antibody prior to subcutaneously administering a treatment dose; and subcutaneously administering step-up doses of 10 μg/kg and 60 μg/kg and 400 μg/kg of the GPRC5D×CD3 bispecific antibody prior to subcutaneously administering a treatment dose.

In certain embodiments, the step-up doses of the BCMA×CD3 bispecific antibody are administered 2-4 days apart from each other, and the step-up doses of the GPRC5D×CD3 bispecific antibody are administered 2-4 days apart from each other.

In certain embodiments, the method comprises:
subcutaneously administering a first step-up dose of 60 μg/kg of the BCMA×CD3 bispecific antibody, then 2-4 days later subcutaneously administering a second step-up dose of 300 μg/kg of the BCMA×CD3 bispecific antibody, then 2-4 days later subcutaneously administering a third step-up dose of 1500 μg/kg of the BCMA×CD3 bispecific antibody, then 2-4 days later subcutaneously administering a first treatment dose of 3000 μg/kg of the BCMA×CD3 bispecific antibody; and
subcutaneously administering a first step-up dose of 10 μg/kg of the GPRC5D×CD3 bispecific antibody, then 2-4 days later subcutaneously administering a second step-up dose of 60 μg/kg of the GPRC5D×CD3 bispecific antibody, then 2-4 days later subcutaneously administering a third step-up dose of 400 μg/kg of the GPRC5D×CD3 bispecific antibody, then 2-4 days later subcutaneously administering a first treatment dose of 800 μg/kg of the GPRC5D×CD3 bispecific antibody, and
thereafter, subcutaneously administering the BCMA×CD3 bispecific antibody every two weeks (Q2W) at a treatment dose of 3000 μg/kg and subcutaneously administering the GPRC5D×CD3 bispecific antibody every two weeks (Q2W) at a treatment dose of 800 μg/kg.

In certain embodiments, each treatment dose of the BCMA×CD3 bispecific antibody is administered on the same day as each treatment dose of the GPRC5D×CD3 bispecific antibody. In certain embodiments, each treatment dose of the BCMA×CD3 bispecific antibody is administered on the same day as each treatment dose of the GPRC5D×CD3 bispecific antibody, at least 15 minutes apart from each other, e.g., about 30 (±10) minutes apart from each other.

In certain embodiments, each step-up dose of the BCMA×CD3 bispecific antibody is administered on the same day as each step-up dose of the GPRC5D×CD3 bispecific antibody. In certain embodiments, each step-up dose of the BCMA×CD3 bispecific antibody is administered on the same day as each step-up dose of the GPRC5D×CD3 bispecific antibody, at least 15 minutes apart from each other, e.g., about 30 (±10) minutes apart from each other.

In certain embodiments, the first treatment dose of the BCMA×CD3 bispecific antibody and the first treatment dose of the GPRC5D×CD3 bispecific antibody are administered subcutaneously on Cycle 1 Day 1 of a 28-day cycle, wherein subsequent treatment doses of the GPRC5D×CD3 bispecific antibody are administered Q2W at a dose amount of 800 μg/kg subcutaneously, e.g., on Days 1 and 15 (±3 days) of each 28-day cycle, and subsequent treatment doses of the BCMA×CD3 bispecific antibody are administered Q2W at a dose amount of 3000 μg/kg subcutaneously, e.g., on Days 1 and 15 (±3 days) of a 28-day cycle.

In certain embodiments, the method comprises subcutaneously administering step-up doses of 60 μg/kg and 300 μg/kg and 1500 μg/kg of the BCMA×CD3 bispecific antibody prior to subcutaneously administering said first treatment dose; and
subcutaneously administering step-up doses of 10 μg/kg and 60 μg/kg and 400 μg/kg of the GPRC5D×CD3 bispecific antibody prior to subcutaneously administering said first treatment dose, wherein said first treatment doses are administered 2 to 4 days after administration of the last step-up dose of each bispecific antibody.

In certain embodiments, the method comprises:
subcutaneously administering step-up doses of 60 μg/kg and 300 μg/kg and 1500 μg/kg of the BCMA×CD3 bispecific antibody prior to subcutaneously administering a first treatment dose of the BCMA×CD3 bispecific antibody; and
subcutaneously administering step-up doses of 10 μg/kg and 60 μg/kg and 400 μg/kg of the GPRC5D×CD3 bispecific antibody prior to subcutaneously administering a first treatment dose of the GPRC5D×CD3 bispecific antibody,
wherein the first treatment dose of the BCMA×CD3 bispecific antibody and the first treatment dose of the GPRC5D×CD3 bispecific antibody are subcutaneously administered on Cycle 1 Day 1 of a 28-day cycle,
wherein subsequent treatment doses of the GPRC5D×CD3 bispecific antibody are administered Q2W at a dose amount of 800 μg/kg subcutaneously. e.g., on Days 1 and 15 (±3 days) of each 28-day cycle, and
subsequent treatment doses of the BCMA×CD3 bispecific antibody are administered Q2W at a dose amount of 3000 μg/kg subcutaneously, e.g., on Days 1 and 15 (±3 days) of a 28-day cycle.

In certain embodiments, the method comprises:
subcutaneously administering a first step-up dose of 60 μg/kg of the BCMA×CD3 bispecific antibody, then 2-4 days later subcutaneously administering a second step-up dose of 300 μg/kg of the BCMA×CD3 bispecific antibody, then 2-4 days later subcutaneously administering a third step-up dose of 1500 μg/kg of the BCMA×CD3 bispecific antibody, then 2-4 days later subcutaneously administering a first treatment dose of the BCMA×CD3 bispecific antibody; and
subcutaneously administering a first step-up dose of 10 μg/kg of the GPRC5D×CD3 bispecific antibody, then 2-4 days later subcutaneously administering a second step-up dose of 60 μg/kg of the GPRC5D×CD3 bispecific antibody, then 2-4 days later subcutaneously administering a third step-up dose of 400 μg/kg of the GPRC5D×CD3 bispecific antibody, then 2-4 days later subcutaneously administering a first treatment dose of the GPRC5D×CD3 bispecific antibody,
wherein the first treatment dose of the BCMA×CD3 bispecific antibody and the first treatment dose of the GPRC5D×CD3 bispecific antibody are subcutaneously administered on Cycle 1 Day 1 of a 28-day cycle,
wherein subsequent treatment doses of the GPRC5D×CD3 bispecific antibody are administered Q2W at a dose amount of 800 μg/kg subcutaneously, e.g., on Days 1 and 15 (±3 days) of each 28-day cycle, and
subsequent treatment doses of the BCMA×CD3 bispecific antibody are administered Q2W at a dose amount of 3000 μg/kg subcutaneously. e.g., on Days 1 and 15 (±3 days) of a 28-day cycle.

In certain embodiments, the method comprises:
subcutaneously administering step-up doses of 60 μg/kg and 300 μg/kg and 1500 μg/kg of the BCMA×CD3 bispecific antibody prior to subcutaneously administering a first treatment dose of the BCMA×CD3 bispecific antibody, and
subcutaneously administering step-up doses of 10 μg/kg and 60 μg/kg and 300 μg/kg of the GPRC5D×CD3 bispecific antibody prior to subcutaneously administering a first treatment dose of the GPRC5D×CD3 bispecific antibody,
wherein the first treatment dose of the BCMA×CD3 bispecific antibody and the first treatment dose of the GPRC5D×CD3 bispecific antibody are subcutaneously administered on Cycle 1 Day 1 of a 28-day cycle,
wherein subsequent treatment doses of the GPRC5D×CD3 bispecific antibody are administered Q2W at a dose amount of 800 μg/kg subcutaneously. e.g., on Days 1 and 15 (±3 days) of each 28-day cycle, and
subsequent treatment doses of the BCMA×CD3 bispecific antibody are administered Q2W at a dose amount of 3000 μg/kg subcutaneously, e.g., on Days 1 and 15 (±3 days) of a 28-day cycle.

In certain embodiments, the method comprises:
subcutaneously administering a first step-up dose of 60 μg/kg of the BCMA×CD3 bispecific antibody, then 2-4 days later subcutaneously administering a second step-up dose of 300 μg/kg of the BCMA×CD3 bispecific antibody, then 2-4 days later subcutaneously administering a third step-up dose of 1500 μg/kg of the BCMA×CD3 bispecific antibody, then 2-4 days later subcutaneously administering a first treatment dose of the BCMA×CD3 bispecific antibody; and
subcutaneously administering a first step-up dose of 10 μg/kg of the GPRC5D×CD3 bispecific antibody, then 2-4 days later subcutaneously administering a second step-up dose of 60 μg/kg of the GPRC5D×CD3 bispecific antibody, then 2-4 days later subcutaneously administering a third step-up dose of 300 μg/kg of the GPRC5D×CD3 bispecific antibody, then 2-4 days later subcutaneously administering a first treatment dose of the GPRC5D×CD3 bispecific antibody,
wherein the first treatment dose of the BCMA×CD3 bispecific antibody and the first treatment dose of the GPRC5D×CD3 bispecific antibody are subcutaneously administered on Cycle 1 Day 1 of a 28-day cycle,
wherein subsequent treatment doses of the GPRC5D×CD3 bispecific antibody are administered Q2W at a dose amount of 800 μg/kg subcutaneously, e.g., on Days 1 and 15 (±3 days) of each 28-day cycle, and
subsequent treatment doses of the BCMA×CD3 bispecific antibody are administered Q2W at a dose amount of 3000 μg/kg subcutaneously, e.g., on Days 1 and 15 (±3 days) of a 28-day cycle.

In certain embodiments, the combination therapy comprises sequential 28-day treatment cycles; administration of the GPRC5D×CD3 bispecific antibody starts in Cycle 1 and administration of the BCMA×CD3 bispecific antibody starts in Cycle 1. According to certain embodiments, a subject is administered the GPRC5D×CD3 bispecific antibody and the BCMA×CD3 bispecific antibody for a finite number of treatment cycles; for example, a subject may be administered the bispecific antibodies for up to 26 cycles if the subject has no sign of progressive disease or toxicity.

The present inventors developed combination dosing regimens for the administration of Tal and Tec based on multiple considerations including, for example, 1) ease and convenience of SC administration compared with IV administration: 2) PK data indicating the selected treatment dose achieved desired target exposure; 3) pharmacodynamic data demonstrating T-cell activation and redistribution; 4) favorable clinical safety profile that appears equivalent to those observed in the lower SC dosing cohorts; 5) a favorable efficacy profile of the selected SC dose; and 6) clinical outcomes of teclistamab in the RedirecTT-1 study.

In part, data in support of the combination of talquetamab and teclistamab in participants with relapsed and refractory multiple myeloma come from the Phase 1 dose escalation study 64007957MMY1003 (RedirecTT-1). In the RedirecTT-1 study, different dose combinations of talquetamab and teclistamab were evaluated. Preliminary data from RedirecTT-1 show that 0.8 mg/kg Q2W of talquetamab and 3 mg/kg Q4W of teclistamab provides robust clinical efficacy with manageable safety profile. Other dosing regimens, such as weekly dosing for both bispecifics were less well-tolerated. Study RedirecTT-1 exploratory E-R analysis demonstrated a trend for improved depth of response associated with Q2W talquetamab dosing in the initial treatment cycles. A reduction in talquetamab dose frequency after response does not compromise efficacy and may ameliorate GPRC5D specific AEs. Monthly administration of teclistamab at 3 mg/kg at Cycle 2 is estimated to achieve comparable exposure to 1.5 mg/kg Q2W based on Study 64007957MMY1001 (MajesTEC-1) and study RedirecTT-1 data. Moreover, there may be improved tolerability with lower frequency dosing while maintaining response.

Data from Study 64407564MMY1001 have shown a median time to first response of 1.2 months and a median time to best response of 2.2 months for participants treated with talquetamab at 0.8 mg/kg SC Q2W. The median concentration-time profile exposure following Q4W dosing is estimated to be at or above the EC90 value identified in an ex vivo cytotoxicity assay and is estimated to be sufficient to maintain efficacy in low tumor burden setting. Thus, in certain embodiments, dosing for talquetamab proceeds in Q4W intervals from Cycle 7 onward (as early as Cycle 5 for participants in confirmed VGPR or better) in order to decrease exposure while maintaining efficacy and improving convenience for participants.

In certain embodiments the method comprises initially administering treatment doses of the BCMA×CD3 bispecific antibody and the GPRC5D×CD3 bispecific antibody on a biweekly (Q2W) dosing schedule and then switching from biweekly dosing to monthly (Q4W) dosing. In certain embodiments, a switch from biweekly to monthly dosing occurs after the subject has achieved a response of VGPR or better and received a minimum of 4 cycles of therapy. In certain embodiments, a switch from biweekly to monthly dosing occurs after the subject has achieved a response of PR or better and received a minimum of 4 cycles of therapy. In certain embodiments, a switch from biweekly to monthly dosing occurs after the subject has completed 6 cycles of treatment, regardless of clinical response.

In certain embodiments, treatment doses of 0.8 mg/kg talquetamab are administered as follows, after a step-up phase: from Cycles 2-4, biweekly (Q2W); from Cycle 5 (C5), if confirmed VGPR or better, schedule can change to Q4W, with the change to Q4W occurring on Cycle 5 or Cycle 6 Day 1 (±3 d); at Cycle 7 Day 1 (±3 d), if confirmed PR or better, schedule must change to Q4W dosing; at Cycle 7 Day 1 (±3 d), if not confirmed PR or better, continue with Q2W dosing until confirmed PR or better is achieved. In certain embodiments, treatment doses of 3 mg/kg teclistamab are administered Q4W from Cycle 2 after a step-up phase. In certain embodiments, the subject is administered a combination regimen comprising teclistamab and talquetamab for 26 treatment cycles (approximately 24 months).

In certain embodiments, talquetamab and teclistamab are administered as follows, in 28-day treatment cycles:
In Cycle 1, the following doses of subcutaneous talquetamab are administered:
   Talquetamab step-up dose 1 (SU1): 0.01 mg/kg, administered on Day 1;
   Talquetamab step-up dose 2 (SU2): 0.06 mg/kg, administered ≥2 d after SU1, between Days 3-6;
   Talquetamab treatment dose (sometimes referred to as step-up dose 3): 0.4 mg/kg, administered ≥2 d after SU2, between Days 5-10;
   Talquetamab treatment dose: 0.8 mg/kg, administered ≥2 d after first treatment dose (step-up dose 3) of 0.4 mg/kg, between Days 7-15;
In subsequent treatment cycles following Cycle 1, the subcutaneous Talquetamab treatment doses of 0.8 mg/kg are administered as follows:
   Cycle 2-Cycle 4 (C2-C4), Talquetamab SC is administered bi-weekly (Q2W), i.e., 14 days (±3 d) after prior treatment dose;
   From Cycle 5 (C5), if confirmed VGPR or better, schedule can change to Q4W dosing (Day 1 of each 28-day cycle only);
   Q4W from Cycle 7 (C7) regardless of clinical response;
In Cycle 1, the following doses of subcutaneous teclistamab are administered:
   Tec step-up dose 1 (SU1): 0.06 mg/kg, administered on Day 1;
   Tec step-up dose 2 (SU2): 0.3 mg/kg, administered ≥2 d after SU1, between Days 3-6;
   Tec treatment dose (sometimes referred to as step-up dose 3): 1.5 mg/kg, administered ≥2 d after SU2, between Days 5-10;
   Tec treatment dose: 3.0 mg/kg, administered ≥2 d after first treatment dose (step-up dose 3) of 1.5 mg/kg, between Days 7-15.
In subsequent treatment cycles following Cycle 1, the subcutaneous Tec treatment doses of 3.0 mg/kg are administered as follows;
   In Cycle 2, Tec is administered 14 days (±3 d) after prior treatment dose;
   From Cycle 3 (C3), if confirmed partial response (PR) or better, schedule can change to Q4W dosing (Day 1 of each 28-day cycle only);
   Q4W from Cycle 7 (C7) regardless of clinical response.

In certain embodiments, a subject is administered talquetamab SC step-up doses at 0.01 and 0.06 mg/kg, 1 dose of 0.4 mg/kg followed by 0.8 mg/kg SC Q2W study treatment dose in combination with teclistamab administered at step-up doses of 0.06 and 0.3, 2 doses of 1.5 mg/kg, and then 3 mg/kg Q4W study treatment dose starting from Cycle 2; from Cycle 5, a subject with response of confirmed VGPR or better can change to talquetamab Q4W′ from Cycle 7, participants with response of confirmed PR or better must be switched to talquetamab 0.8 mg/kg Q4W.

In certain embodiments, talquetamab and teclistamab are administered as follows, in 28-day treatment cycles:
In Cycle 1, the following doses of subcutaneous talquetamab are administered:
   Talquetamab step-up dose 1 (SU1): 0.01 mg/kg, administered on Day 1;
   Talquetamab step-up dose 2 (SU2): 0.06 mg/kg, administered ≥2 d after SU1;
   Talquetamab treatment dose (sometimes referred to as step-up dose 3): 0.4 mg/kg, administered ≥2 d after SU2;
   Talquetamab treatment dose: 0.8 mg/kg, administered ≥2 d after first treatment dose (step-up dose 3) of 0.4 mg/kg, between Days 7-15,
In subsequent treatment cycles following Cycle 1, the subcutaneous Talquetamab treatment doses of 0.8 mg/kg are administered as follows:
   Cycle 2-Cycle 4 (C2-C4). Talquetamab SC is administered bi-weekly (Q2W), i.e., 14 days (±3 d) after prior treatment dose;
   From Cycle 5 (C5), if confirmed VGPR or better, schedule can change to Q4W dosing (Day 1 of each 28-day cycle only), with the change to Q4W occurring on Cycle 5 or Cycle 6 Day 1 (±3 d);
   At Cycle 7 Day 1 (±3 d), if confirmed PR or better, schedule must change to Q4W dosing;
   At Cycle 7 Day 1 (±3 d), if not confirmed PR or better, continue with Q2W dosing until confirmed PR or better is achieved.
In Cycle 1, the following doses of subcutaneous teclistamab are administered:
   Tec step-up dose 1 (SU1): 0.06 mg/kg, administered on Day 1;
   Tec step-up dose 2 (SU2): 0.3 mg/kg, administered ≥2 d after SU1:
   Tec treatment dose (sometimes referred to as step-up dose 3): 1.5 mg/kg, administered ≥2 d after SU2;
   Tec treatment dose: 1.5 mg/kg, administered 22 d after first treatment dose (step-up dose 3) of 1.5 mg/kg, between Days 7-15.
In subsequent treatment cycles following Cycle 1, the subcutaneous Tec treatment doses of 3.0 mg/kg are administered as follows.
   From Cycle 2 (C2), Q4W dosing (Day 1 only) regardless of clinical response.

In certain embodiments, the subject has received between 1-4 prior lines of therapy including an anti-CD38 antibody (e.g., daratumumab) and lenalidomide, and the method comprises:
treating the subject according to a therapeutically effective regimen that comprises sequential 28-day treatment cycles, wherein:
step-up doses of the GPRC5D×CD3 bispecific antibody (e.g., talquetamab) and the BCMA×CD3 bispecific antibody (e.g., teclistamab) are subcutaneously administered to the subject during a step-up phase in Cycle 1, and then:
treatment doses of the BCMA×CD3 bispecific antibody are subcutaneously administered to the subject, each in an amount of 3.0 mg/kg, on a monthly dosing schedule (Q4W) starting from treatment Cycle 2, regardless of clinical response in the subject; and
treatment doses of the GPRC5D×CD3 bispecific antibody are subcutaneously administered to the subject, each in an amount of 0.8 mg/on a bi-weekly dosing schedule (Q2W) starting from the treatment Cycle 2 (e.g., on Days 1 and 15), and then treatment doses of the GPRC5D×CD3 bispecific antibody are subcutaneously administered to the subject on a monthly dosing schedule (Q4W) either: (i) starting at treatment Cycle 5 if the subject has achieved a very good partial response, a complete response or a stringent complete response, as determined by IMWG response criteria; or (ii) starting at treatment Cycle 7 if the subject has achieved a partial response, a very good partial response, a complete response or a stringent complete response, as determined by IMWG response criteria; or (iii) after treatment Cycle 7 when a partial response, a very good partial response, a complete response or a stringent complete response is achieved, as determined by IMWG response criteria.

In certain embodiments, a method of improving median progression free survival (PFS) in a population of subjects with relapsed or refractory multiple myeloma that have received between 1-4 prior lines of therapy including an anti-CD38 antibody (e.g., daratumumab) and lenalidomide, comprises administering to the population of subjects a combination therapy comprising a therapeutically effective amount of a GPRC5D×CD3 bispecific antibody (e.g., talquetamab) and a therapeutically effective amount of a BCMA×CD3 bispecific antibody (e.g., teclistamab), wherein the method comprises:
treating the subject according to a therapeutically effective regimen that comprises sequential 28-day treatment cycles, wherein:
step-up doses of the GPRC5D×CD3 bispecific antibody (e.g., talquetamab) and the BCMA×CD3 bispecific antibody (e.g., teclistamab) are subcutaneously administered to the subject during a step-up phase in Cycle 1, and then
treatment doses of the BCMA×CD3 bispecific antibody are subcutaneously administered to the subject, each in an amount of 3.0 mg/kg, on a monthly dosing schedule (Q4W) starting from treatment Cycle 2, regardless of clinical response in the subject; and
treatment doses of the GPRC5D×CD3 bispecific antibody are subcutaneously administered to the subject, each in an amount of 0.8 mg/on a bi-weekly dosing schedule (Q2W) starting from treatment Cycle 2 (e.g., on Days 1 and 15), and then treatment doses of the GPRC5D×CD3 bispecific antibody are subcutaneously administered to the subject on a monthly dosing schedule (Q4W) either: (i) starting at treatment Cycle 5 if the subject has achieved a very good partial response, a complete response or a stringent complete response, as determined by IMWG response criteria; or (ii) starting at treatment Cycle 7 if the subject has achieved a partial response, a very good partial response, a complete response or a stringent complete response, as determined by IMWG response criteria; or (iii) after treatment Cycle 7 when a partial response, a very good partial response, a complete response or a stringent complete response is achieved, as determined by IMWG response criteria,
wherein the improvement in median PFS is relative to median PFS of a reference population of subjects with relapsed or refractory multiple myeloma that have received between 1-4 prior lines of therapy including an anti-CD38 antibody (e.g., daratumumab) and lenalidomide, said reference population having been administered either (i) elotuzumab, pomalidomide, and dexamethasone (EPd) or (ii) pomalidomide, bortezomib, and dexamethasone (PVd).

In certain embodiments, a method of improving overall response rate (ORR) in a population of subjects with relapsed or refractory multiple myeloma that have received between 1-4 prior lines of therapy including an anti-CD38 antibody (e.g., daratumumab) and lenalidomide, comprises administering to the population of subjects a combination therapy comprising a therapeutically effective amount of a GPRC5D×CD3 bispecific antibody (e.g., talquetamab) and a therapeutically effective amount of a BCMA×CD3 bispecific antibody (e.g., teclistamab), wherein the method comprises:
treating the subjects according to a therapeutically effective regimen that comprises sequential 28-day treatment cycles, wherein:
step-up doses of the GPRC5D×CD3 bispecific antibody (e.g., talquetamab) and the BCMA×CD3 bispecific antibody (e.g., teclistamab) are subcutaneously administered to the subject during a step-up phase in Cycle 1, and then
treatment doses of the BCMA×CD3 bispecific antibody are subcutaneously administered to the subject, each in an amount of 3.0 mg/kg, on a monthly dosing schedule (Q4W) starting from treatment Cycle 2, regardless of clinical response in the subject; and
treatment doses of the GPRC5D×CD3 bispecific antibody are subcutaneously administered to the subject, each in an amount of 0.8 mg/on a bi-weekly dosing schedule (Q2W) starting from treatment Cycle 2 (e.g., on Days 1 and 15), and then treatment doses of the GPRC5D×CD3 bispecific antibody are subcutaneously administered to the subject on a monthly dosing schedule (Q4W) either: (i) starting at treatment Cycle 5 if the subject has achieved a very good partial response, a complete response or a stringent complete response, as determined by IMWG response criteria; or (ii) starting at treatment Cycle 7 if the subject has achieved a partial response, a very good partial response, a complete response or a stringent complete response, as determined by IMWG response criteria; or (iii) after treatment Cycle 7 when a partial response, a very good partial response, a complete response or a stringent complete response is achieved, as determined by IMWG response criteria.
wherein the improvement in ORR is relative to ORR of a reference population of subjects with relapsed or refractory multiple myeloma that have received between 1-4 prior lines of therapy including an anti-CD38 antibody (e.g., daratumumab) and lenalidomide, said reference population having been administered either (i) elotuzumab, pomalidomide, and dexamethasone (EPd) or (ii) pomalidomide, bortezomib, and dexamethasone (PVd)

In certain embodiments, the step-up phase comprises subcutaneously administering step-up doses of 0.01 mg/kg and 0.06 mg/kg of the GPRC5D×CD3 bispecific antibody 2-4 days apart from each other (e.g., on Days 1 and 4, respectively). In certain embodiments, the step-up phase comprises subcutaneously administering step-up doses of 0.01 mg/kg, 0.06 mg/kg and 0.4 mg/kg of the GPRC5D×CD3 bispecific antibody 2-4 days apart from each other (e.g., on Days 1, 4 and 8, respectively). In certain embodiments, the step-up phase comprises subcutaneously administering step-up doses of 0.01 mg/kg, 0.06 mg/kg and 0.4 mg/kg of the GPRC5D×CD3 bispecific antibody 2-4 days apart from each other (e.g., on Days 1, 4 and 8, respectively) and then subcutaneously administering the treatment dose of 0.8 mg/kg of the GPRC5D×CD3 bispecific antibody at least two days after the 0.4 mg/kg dose (e.g., between Days 7-15, such as Day 15).

In certain embodiments, the step-up phase comprises subcutaneously administering step-up doses of 0.06 mg/kg and 0.3 mg/kg of the BCMA×CD3 bispecific antibody 2-4 days apart from each other (e.g., on Days 1 and 4, respectively). In certain embodiments, the step-up phase comprises subcutaneously administering step-up doses of 0.06 mg/kg, 0.3 mg/kg and 1.5 mg/kg of the BCMA×CD3 bispecific antibody 2-4 days apart from each other (e.g., on Days 1, 4 and 8, respectively). In certain embodiments, the step-up phase comprises subcutaneously administering step-up doses of 0.06 mg/kg, 0.3 mg/kg and 1.5 mg/kg of the BCMA×CD3 bispecific antibody 2-4 days apart from each other (e.g., on Days 1, 4 and 8, respectively) and then subcutaneously administering a dose of 1.5 mg/kg of the BCMA×CD3 bispecific antibody at least two days after the 1.5 mg/kg step-up dose (e.g., between Days 7-15, such as Day 15) In certain embodiments, the GPRC5D×CD3 bispecific antibody (e.g., talquetamab) and the BCMA×CD3 bispecific antibody (e.g., teclistamab) are administered at least 15 minutes apart from each other each day that doses are administered (e.g., the GPRC5D×CD3 bispecific antibody is administered first).

In certain embodiments, the combination regimen has a finite duration of 26 treatment cycles.

In alternative embodiments, the method comprises:
subcutaneously administering to the subject step-up doses of the BCMA×CD3 bispecific antibody (e.g., teclistamab) and the GPRC5D×CD3 bispecific antibody (e.g., talquetamab), and then
subcutaneously administering treatment doses of 3000 μg/kg of the BCMA×CD3 bispecific antibody and 800 μg/kg of the GPRC5D×CD3 bispecific antibody biweekly (Q2W) for at least four 28-day cycles (e.g., on Days 1 and 15 (±3 days) of each cycle); and
(i) subcutaneously administering the treatment doses of 3000 μg/kg of the BCMA×CD3 bispecific antibody and 800 μg/kg of the GPRC5D×CD3 bispecific antibody monthly (Q4W) (e.g., on Day 1 of each 28-day cycle) when the subject has achieved a response of VGPR or better and received a minimum of four 28-day cycles of treatment (e.g., starting on Cycle 5); or
(ii) subcutaneously administering the treatment doses of 3000 μg/kg of the BCMA×CD3 bispecific antibody and 800 μg/kg of the GPRC5D×CD3 bispecific antibody monthly (Q4W) (e.g., on Day 1 of each 28-day cycle), regardless of clinical response in the subject, after the subject has completed 6 28-day cycles of treatment (e.g., starting on Cycle 7).

In certain embodiments, doses of teclistamab and talquetamab are administered on the same day, at least 15 minutes apart from each other. In certain embodiments, doses of teclistamab and talquetamab are administered about 20-40 minutes apart from each other, e.g., within about 30 minutes of each other. In certain embodiments, teclistamab is administered before talquetamab, e.g., at least 15 minutes before talquetamab, or about 30 minutes before talquetamab. In other embodiments, talquetamab is administered before teclistamab, e.g., at least 15 minutes before teclistamab, or about 30 minutes before teclistamab.

In certain embodiments, the method achieves a partial response, a very good partial response, a complete response or a stringent complete response in the subject, as determined by IMWG response criteria.

In certain embodiments, the method achieves an overall response rate of at least 50% in a population of subjects with relapsed or refractory multiple myeloma (RRMM), e.g., in subjects with EMD.

In certain embodiments, the method achieves an overall response rate of at least 55% in a population of subjects with relapsed or refractory multiple myeloma (RRMM), e.g., in subjects with EMD.

In certain embodiments, the method achieves an overall response rate of at least 60% in a population of subjects with relapsed or refractory multiple myeloma (RRMM), e.g., in subjects with EMD.

In certain embodiments, the method achieves an overall response rate of at least 65% in a population of subjects with relapsed or refractory multiple myeloma (RRMM), e.g., in subjects with EMD.

In certain embodiments, the method achieves an overall response rate of at least 70% in a population of subjects with relapsed or refractory multiple myeloma (RRMM), e.g., in subjects with EMD.

In certain embodiments, the method achieves an overall response rate of at least 80% in a population of subjects with RRMM, e.g., in subjects with EMD.

In certain embodiments, the method achieves an overall response rate of at least 85% in a population of subjects with RRMM, e.g., in subjects with EMD.

In certain embodiments, the method achieves an overall response rate of at least 90% in a population of subjects with RRMM.

In certain embodiments, the method achieves an overall response rate of at least 95% in a population of subjects with RRMM.

In certain embodiments, the method achieves a complete response or a stringent complete response rate of at least 20% in a population of subjects with RRMM, e.g., in subjects with EMD.

In certain embodiments, the method achieves a complete response or a stringent complete response rate of at least 25% in a population of subjects with RRMM, e.g., in subjects with EMD.

In certain embodiments, the method achieves a complete response or a stringent complete response rate of at least 30% in a population of subjects with RRMM, e.g., in subjects with EMD.

In certain embodiments, the method achieves a complete response or a stringent complete response rate of at least 35% in a population of subjects with RRMM. e.g., in subjects with EMD.

In certain embodiments, the method achieves a complete response or a stringent complete response rate of at least 40% in a population of subjects with RRMM, e.g., in subjects with EMD.

In certain embodiments, the subject has received between 1-4 prior lines of therapy including an anti-CD38 antibody (e.g., daratumumab) and lenalidomide, and the method comprises: treating the subject according to a therapeutically effective regimen that comprises sequential 28-day treatment cycles, wherein: one or more step-up doses of the GPRC5D×CD3 bispecific antibody (e.g., talquetamab) are subcutaneously administered to the subject during a step-up phase in Cycle 1, and then each treatment dose of the GPRC5D×CD3 bispecific antibody is subcutaneously administered to the subject in an amount of 0.8 mg/kg, wherein the treatment doses of the GPRC5D×CD3 bispecific antibody are administered to the subject on a bi-weekly dosing schedule (Q2W) starting from treatment Cycle 2 (e.g., on Days 1 and 15), and then treatment doses of the GPRC5D×CD3 bispecific antibody are subcutaneously administered to the subject on a monthly dosing schedule (Q4W) either: (i) starting at treatment Cycle 5 if the subject has achieved a very good partial response, a complete response or a stringent complete response, as determined by IMWG response criteria; or (ii) starting at treatment Cycle 7, regardless of clinical response in the subject.

In certain embodiments, one or more step-up doses of the BCMA×CD3 bispecific antibody (e.g., teclistamab) are subcutaneously administered to the subject during a step-up phase in Cycle 1, and each treatment dose of the BCMA×CD3 bispecific antibody is subcutaneously administered to the subject in an amount of 3.0 mg/kg, wherein the treatment doses of the BCMA×CD3 bispecific antibody are administered to the subject on a bi-weekly dosing schedule (Q2W) starting from treatment Cycle 2 (e.g., on Days 1 and 15), and then treatment doses of the BCMA×CD3 bispecific antibody are subcutaneously administered to the subject on a monthly dosing schedule (Q4W) either: (i) starting at treatment Cycle 3 if the subject has achieved a partial response, a very good partial response, a complete response or a stringent complete response, as determined by IMWG response criteria; or (ii) starting at treatment Cycle 7, regardless of clinical response in the subject.

In certain embodiments, a method of improving median progression free survival (PFS) in a population of subjects with relapsed or refractory multiple myeloma that have received between 1-4 prior lines of therapy including an anti-CD38 antibody (e.g., daratumumab) and lenalidomide, comprises administering to the population of subjects a combination therapy comprising a therapeutically effective amount of a GPRC5D×CD3 bispecific antibody (e.g., talquetamab) and a therapeutically effective amount of a BCMA×CD3 bispecific antibody (e.g., teclistamab), wherein the improvement in median PFS is relative to median PFS of a reference population of subjects with relapsed or refractory multiple myeloma that have received between 1-4 prior lines of therapy including an anti-CD38 antibody (e.g., daratumumab) and lenalidomide, said reference population having been administered either (i) elotuzumab, pomalidomide, and dexamethasone (EPd) or (ii) pomalidomide, bortezomib, and dexamethasone (PVd). According to certain embodiments, the method comprises: treating the subject according to a therapeutically effective regimen that comprises sequential 28-day treatment cycles, wherein: one or more step-up doses of the GPRC5D×CD3 bispecific antibody are subcutaneously administered to the subject during a step-up phase in Cycle 1, and then each treatment dose of the GPRC5D×CD3 bispecific antibody is subcutaneously administered to the subject in an amount of 0.8 mg/kg, wherein the treatment doses of the GPRC5D×CD3 bispecific antibody are administered to the subject on a bi-weekly dosing schedule (Q2W) starting from treatment Cycle 2 (e.g., on Days 1 and 15), and then treatment doses of the GPRC5D×CD3 bispecific antibody are subcutaneously administered to the subject on a monthly dosing schedule (Q4W) either: (i) starting at treatment Cycle 5 if the subject has achieved a very good partial response, a complete response or a stringent complete response, as determined by IMWG response criteria; or (ii) starting at treatment Cycle 7, regardless of clinical response in the subject, and wherein one or more step-up doses of the BCMA×CD3 bispecific antibody (e.g., teclistamab) are subcutaneously administered to the subject during a step-up phase in Cycle 1, and each treatment dose of the BCMA×CD3 bispecific antibody is subcutaneously administered to the subject in an amount of 3.0 mg/kg, wherein the treatment doses of the BCMA×CD3 bispecific antibody are administered to the subject on a bi-weekly dosing schedule (Q2W) starting from treatment Cycle 2 (e.g., on Days 1 and 15), and then treatment doses of the BCMA×CD3 bispecific antibody are subcutaneously administered to the subject on a monthly dosing schedule (Q4W) either: (i) starting at treatment Cycle 3 if the subject has achieved a partial response, a very good partial response, a complete response or a stringent complete response, as determined by IMWG response criteria; or (ii) starting at treatment Cycle 7, regardless of clinical response in the subject.

In certain embodiments, a method of improving ORR in a population of subjects with relapsed or refractory multiple myeloma that have received between 1-4 prior lines of therapy including an anti-CD38 antibody (e.g., daratumumab) and lenalidomide, comprises administering to the population of subjects a combination therapy comprising a therapeutically effective amount of a GPRC5D×CD3 bispecific antibody (e.g., talquetamab) and a therapeutically effective amount of a BCMA×CD3 bispecific antibody (e.g., teclistamab), wherein the improvement in ORR is relative to ORR of a reference population of subjects with relapsed or refractory multiple myeloma that have received between 1-4 prior lines of therapy including an anti-CD38 antibody (e.g., daratumumab) and lenalidomide, said reference population having been administered either (i) elotuzumab, pomalidomide, and dexamethasone (EPd) or (ii) pomalidomide, bortezomib, and dexamethasone (PVd). According to certain embodiments, the method comprises: treating the subject according to a therapeutically effective regimen that comprises sequential 28-day treatment cycles, wherein: one or more step-up doses of the GPRC5D×CD3 bispecific antibody are subcutaneously administered to the subject during a step-up phase in Cycle 1, and then each treatment dose of the GPRC5D×CD3 bispecific antibody is subcutaneously administered to the subject in an amount of 0.8 mg/kg, wherein the treatment doses of the GPRC5D×CD3 bispecific antibody are administered to the subject on a bi-weekly dosing schedule (Q2W) starting from treatment Cycle 2 (e.g., on Days 1 and 15), and then treatment doses of the GPRC5D×CD3 bispecific antibody are subcutaneously administered to the subject on a monthly dosing schedule (Q4W) either; (i) starting at treatment Cycle 5 if the subject has achieved a very good partial response, a complete response or a stringent complete response, as determined by IMWG response criteria; or (ii) starting at treatment Cycle 7, regardless of clinical response in the subject, and wherein one or more step-up doses of the BCMA×CD3 bispecific antibody (e.g., teclistamab) are subcutaneously administered to the subject during a step-up phase in Cycle 1, and each treatment dose of the BCMA×CD3 bispecific antibody is subcutaneously administered to the subject in an amount of 3.0 mg/kg, wherein the treatment doses of the BCMA×CD3 bispecific antibody are administered to the subject on a bi-weekly dosing schedule (Q2W) starting from treatment Cycle 2 (e.g., on Days 1 and 15), and then treatment doses of the BCMA×CD3 bispecific antibody are subcutaneously administered to the subject on a monthly dosing schedule (Q4W) either: (i) starting at treatment Cycle 3 if the subject has achieved a partial response, a very good partial response, a complete response or a stringent complete response, as determined by IMWG response criteria; or (ii) starting at treatment Cycle 7, regardless of clinical response in the subject.

### EXEMPLARY EMBODIMENTS

Provided below are enumerated embodiments of the present invention. These embodiments are illustrative only and do not limit the scope of the present disclosure or of the claims attached hereto.
1A. A method of treating multiple myeloma in a subject in need thereof, comprising administering to the subject a combination dosing regimen comprising a therapeutically effective amount of a BCMA×CD3 bispecific antibody (e.g., teclistamab) and a therapeutically effective amount of a GPRC5D×CD3 bispecific antibody (e.g., talquetamab), wherein the subject has relapsed or refractory multiple myeloma and received at least three prior lines of therapy, including a proteasome inhibitor, an immunomodulatory agent and an anti-CD38 monoclonal antibody.
2A. The method of embodiment 1A, wherein the BCMA×CD3 bispecific antibody comprises a BCMA binding domain comprising the HCDR1 of SEQ ID NO: 4, the HCDR2 of SEQ ID NO: 5, the HCDR3 of SEQ ID NO: 6, the LCDR1 of SEQ ID NO: 7, the LCDR2 of SEQ ID NO: 8 and the LCDR3 of SEQ ID NO: 9, and a CD3 binding domain comprising the HCDR1 of SEQ ID NO: 14, the HCDR2 of SEQ ID NO: 15, the HCDR3 of SEQ ID NO: 16, the LCDR1 of SEQ ID NO: 17, the LCDR2 of SEQ ID NO: 18 and the LCDR3 of SEQ ID NO: 19.
3A. The method of embodiment 2A, wherein the BCMA binding domain comprises a heavy chain variable region (VH) having the amino acid sequence of SEQ ID NO: 10 and a light chain variable region (VL) having the amino acid sequence of SEQ ID NO: 11, and the CD3 binding domain comprises a heavy chain variable region (VH) having the amino acid sequence of SEQ ID NO: 20 and a light chain variable region (VL) having the amino acid sequence of SEQ ID NO: 21.
4A. The method of any of embodiments 1A-3A, wherein the BCMA×CD3 bispecific antibody comprises a first heavy chain (HC1) having the amino acid sequence of SEQ ID NO: 12, a first light chain (LC1) having the amino acid sequence of SEQ ID NO: 13, a second heavy chain (HC2) having the amino acid sequence of SEQ ID NO: 22 and a second light chain (LC2) having the amino acid sequence of SEQ ID NO: 23.
5A. The method of any of embodiments 1A-4A, wherein the BCMA×CD3 bispecific antibody is teclistamab.
6A. The method of any of embodiments 1A-5A, wherein the GPRC5D×CD3 bispecific antibody comprises a GPRC5D binding domain comprising the HCDR1 of SEQ ID NO: 24, the HCDR2 of SEQ ID NO: 25, the HCDR3 of SEQ ID NO: 26, the LCDR1 of SEQ ID NO: 27, the LCDR2 of SEQ ID NO: 28 and the LCDR3 of SEQ ID NO: 29, and a CD3 binding domain comprising the HCDR1 of SEQ ID NO: 14, the HCDR2 of SEQ ID NO: 15, the HCDR3 of SEQ ID NO: 16, the LCDR1 of SEQ ID NO: 17, the LCDR2 of SEQ ID NO: 18 and the LCDR3 of SEQ ID NO: 19.
7A. The method of embodiment 6A, wherein the GPRC5D binding domain comprises a heavy chain variable region (VH) having the amino acid sequence of SEQ ID NO: 30 and a light chain variable region (VL) having the amino acid sequence of SEQ ID NO: 31, and the CD3 binding domain comprises a heavy chain variable region (VH) having the amino acid sequence of SEQ ID NO: 20 and a light chain variable region (VL) having the amino acid sequence of SEQ ID NO: 21.
8A. The method of any of embodiments 1A-7A, wherein the GPRC5D×CD3 bispecific antibody comprises a first heavy chain (HC1) having the amino acid sequence of SEQ ID NO: 32, a first light chain (LC1) having the amino acid sequence of SEQ ID NO: 33, a second heavy chain (HC2) having the amino acid sequence of SEQ ID NO: 22 and a second light chain (LC2) having the amino acid sequence of SEQ ID NO: 23.
9A. The method of any of embodiments 1A-8A, wherein the GPRC5D×CD3 bispecific antibody is talquetamab.
10A. The method of any of embodiments 1A-9A, wherein the subject has extramedullary disease (EMD).
11A. The method of any of embodiments 1A-9A, wherein the method achieves an overall response rate of at least about 80% in subjects with EMD.
12A. The method of any of embodiments 1A-9A, wherein the method achieves an overall response rate of at least about 90%.
13A. The method of any of embodiments 1A-12A comprising subcutaneously administering the BCMA×CD3 bispecific antibody every two weeks (Q2W) at a treatment dose of 3000 μg/kg and subcutaneously administering the GPRC5D×CD3 bispecific antibody every two weeks (Q2W) at a treatment dose of 800 μg/kg.
14A. The method of any of embodiments 1A-12A comprising subcutaneously administering the BCMA×CD3 bispecific antibody every two weeks (Q2W) at a treatment dose of 1500 μg/kg and subcutaneously administering the GPRC5D×CD3 bispecific antibody every two weeks (Q2W) at a treatment dose of 80 μg/kg.
15A. The method of any of embodiments 1A-12A comprising subcutaneously administering the BCMA×CD3 bispecific antibody weekly (QW) at a treatment dose of 1500 μg/kg and subcutaneously administering the GPRC5D×CD3 bispecific antibody every two weeks (Q2W) at a treatment dose of 800 μg/kg.
16A. The method of any of embodiments 1A-12A comprising subcutaneously administering the BCMA×CD3 bispecific antibody weekly (QW) at a treatment dose of 1500 μg/kg and subcutaneously administering the GPRC5D×CD3 bispecific antibody weekly (QW) at a treatment dose of 400 μg/kg.
17A. The method of any of embodiments 13A-16A comprising subcutaneously administering one or more step-up doses of the BCMA×CD3 bispecific antibody prior to subcutaneously administering a treatment dose; and subcutaneously administering one or more step-up doses of the GPRC5D×CD3 bispecific antibody prior to subcutaneously administering a treatment dose.
18A. The method of any of embodiments 13A-16A comprising subcutaneously administering 2-3 step-up doses of the BCMA×CD3 bispecific antibody prior to subcutaneously administering a treatment dose; and subcutaneously administering 2-3 step-up doses of the GPRC5D×CD3 bispecific antibody prior to subcutaneously administering a treatment dose.
19A. The method of any of embodiments 13A-16A comprising subcutaneously administering step-up doses of 60 μg/kg and 300 μg/kg and 1500 μg/kg of the BCMA×CD3 bispecific antibody prior to subcutaneously administering a treatment dose; and subcutaneously administering step-up doses of 10 μg/kg and 60 μg/kg and 300 μg/kg of the GPRC5D×CD3 bispecific antibody prior to subcutaneously administering a treatment dose.
20A. The method of any of embodiments 13A-16A comprising subcutaneously 30 administering step-up doses of 60 μg/kg and 300 μg/kg and 1500 μg/kg of the BCMA×CD3 bispecific antibody prior to subcutaneously administering a treatment dose; and subcutaneously administering step-up doses of 10 μg/kg and 60 μg/kg and 400 μg/kg of the GPRC5D×CD3 bispecific antibody prior to subcutaneously administering a treatment dose.
21A. The method of any of embodiments 17A-20A, wherein the step-up doses are administered 2-4 days apart.
22A. The method of any of embodiments 13A-21A, wherein each treatment dose of the BCMA×CD3 bispecific antibody is administered on the same day as each treatment dose of the GPRC5D×CD3 bispecific antibody.
23A. A combination dosing regimen for treatment of patients with relapsed or refractory multiple myeloma, the combination dosing regimen comprising administration of a BCMA×CD3 bispecific antibody (e.g., teclistamab) and a GPRC5D×CD3 bispecific antibody (e.g., talquetamab) in amounts effective to achieve an overall response rate of at least about 80%.

Additional enumerated embodiments of the present invention are provided below. These embodiments are illustrative only and do not limit the scope of the present disclosure or of the claims attached hereto.
1. A method of treating multiple myeloma in a subject in need thereof, comprising administering to the subject a combination dosing regimen comprising a therapeutically effective amount of a BCMA×CD3 bispecific antibody and a therapeutically effective amount of a GPRC5D×CD3 bispecific antibody.
2. The method of embodiment 1, wherein the BCMA×CD3 bispecific antibody comprises a BCMA binding domain comprising the HCDR1 of SEQ ID NO: 4, the HCDR2 of SEQ ID NO: 5, the HCDR3 of SEQ ID NO: 6, the LCDR1 of SEQ ID NO: 7, the LCDR2 of SEQ ID NO: 8 and the LCDR3 of SEQ ID NO: 9, and a CD3 binding domain comprising the HCDR1 of SEQ ID NO: 14, the HCDR2 of SEQ ID NO: 15, the HCDR3 of SEQ ID NO: 16, the LCDR1 of SEQ ID NO: 17, the LCDR2 of SEQ ID NO: 18 and the LCDR3 of SEQ ID NO: 19.
3. The method of embodiment 1 or 2, wherein the BCMA×CD3 bispecific antibody comprises a BCMA binding domain comprising a heavy chain variable region (VH) having the amino acid sequence of SEQ ID NO: 10 and a light chain variable region (VL) having the amino acid sequence of SEQ ID NO: 11, and the CD3 binding domain comprises a heavy chain variable region (VH) having the amino acid sequence of SEQ ID NO: 20 and a light chain variable region (VL) having the amino acid sequence of SEQ ID NO: 21.
4. The method of any of embodiments 1-3, wherein the BCMA×CD3 bispecific antibody is an IgG1, an IgG2, an IgG3 or an IgG4 isotype.
5. The method of any of embodiments 1-4, wherein the BCMA×CD3 bispecific antibody is an IgG4 isotype.
6. The method of any of embodiments 1-5, wherein the BCMA×CD3 bispecific antibody comprises one or more substitutions in its Fc region.
7. The method of any of embodiments 1-6, wherein the BCMA×CD3 bispecific antibody is an IgG4 isotype and comprises S228P, F234A and L235A substitutions in its Fc region.
8. The method of any of embodiments 1-7, wherein the BCMA×CD3 bispecific antibody is an IgG4 isotype and comprises S228P, F234A, L235A F405L and R409K substitutions in its Fc region.
9. The method of any of embodiments 1-8, wherein the Fc region of the BCMA-specific IgG4 antibody from which the BCMA-binding arm is derived comprises S228P, L234A and L235A substitutions in its Fc region.
10. The method of any of embodiments 1-9, wherein the Fc region of the CD3-specific IgG4 antibody from which the CD3-binding arm is derived comprises S228P, L234A, L235A. F405L, and R409K substitutions in its Fc region.
11. The method of any of embodiments 1-10, wherein the BCMA×CD3 bispecific antibody comprises a first heavy chain (HC1) having the amino acid sequence of SEQ ID NO: 12, a first light chain (LC1) having the amino acid sequence of SEQ ID NO: 13, a second heavy chain (HC2) having the amino acid sequence of SEQ ID NO: 22 and a second light chain (LC2) having the amino acid sequence of SEQ ID NO: 23.
12. The method of any of embodiments 1-10, wherein the BCMA×CD3 bispecific antibody comprises a first heavy chain (HC1) having at least 90% identity to the amino acid sequence of SEQ ID NO: 12, a first light chain (LC1) having at least 90% identity to the amino acid sequence of SEQ ID NO: 13, a second heavy chain (HC2) having at least 90% identity to the amino acid sequence of SEQ ID NO: 22 and a second light chain (LC2) having at least 90% identity to the amino acid sequence of SEQ ID NO: 23.
13. The method of any of embodiments 1-10, wherein the BCMA×CD3 bispecific antibody comprises a first heavy chain (HC1) having at least 95% identity to the amino acid sequence of SEQ ID NO: 12, a first light chain (LC1) having at least 95% identity to the amino acid sequence of SEQ ID NO: 13, a second heavy chain (HC2) having at least 95% identity to the amino acid sequence of SEQ ID NO: 22 and a second light chain (LC2) having at least 95% identity to the amino acid sequence of SEQ ID NO: 23.
14. The method of any of embodiments 1-10, wherein the BCMA×CD3 bispecific antibody comprises a first heavy chain (HC1) having at least 98% identity to the amino acid sequence of SEQ ID NO: 12, a first light chain (LC1) having at least 98% identity to the amino acid sequence of SEQ ID NO: 13, a second heavy chain (HC2) having at least 98% identity to the amino acid sequence of SEQ ID NO: 22 and a second light chain (LC2) having at least 98% identity to the amino acid sequence of SEQ ID NO: 23.
15. The method of any of embodiments 1-10, wherein the BCMA×CD3 bispecific antibody is teclistamab.
16. The method of any of embodiments 1-15, wherein the GPRC5D×CD3 bispecific antibody comprises a GPRC5D binding domain comprising the HCDR1 of SEQ ID NO: 24, the HCDR2 of SEQ ID NO: 25, the HCDR3 of SEQ ID NO: 26, the LCDR1 of SEQ ID NO: 27, the LCDR2 of SEQ ID NO: 28 and the LCDR3 of SEQ ID NO: 29, and a CD3 binding domain comprising the HCDR1 of SEQ ID NO: 14, the HCDR2 of SEQ ID NO: 15, the HCDR3 of SEQ ID NO: 16, the LCDR1 of SEQ ID NO: 17, the LCDR2 of SEQ ID NO: 18 and the LCDR3 of SEQ ID NO: 19.
17. The method of any of embodiments 1-16, wherein the GPRC5D×CD3 bispecific antibody comprises a GPRC5D binding domain comprising a heavy chain variable region (VH) having the amino acid sequence of SEQ ID NO: 30 and a light chain variable region (VL) having the amino acid sequence of SEQ ID NO: 31, and the CD3 binding domain comprises a heavy chain variable region (VH) having the amino acid sequence of SEQ ID NO: 20 and a light chain variable region (VL) having the amino acid sequence of SEQ ID NO: 21.
18. The method of any of embodiments 1-17, wherein the GPRC5D×CD3 bispecific antibody is an IgG1, an IgG2, an IgG3 or an IgG4 isotype.
19. The method of any of embodiments 1-18, wherein the GPRC5D×CD3 bispecific antibody is an IgG4 isotype.
20. The method of any of embodiments 1-19, wherein the GPRC5D×CD3 bispecific antibody comprises one or more substitutions in its Fc region.
21. The method of any of embodiments 1-20, wherein the GPRC5D×CD3 bispecific antibody is an IgG4 isotype and comprises S228P. F234A and L235A substitutions in its Fc region.
22. The method of any of embodiments 1-21, wherein the GPRC5D×CD3 bispecific antibody is an IgG4 isotype and comprises S228P, F234A, L235A F405L and R409K substitutions in its Fc region.
23. The method of any of embodiments 1-22, wherein the Fc region of the GPRC5D-specific IgG4 antibody from which the GPRC5D-binding arm is derived comprises S228P, L234A and L235A substitutions in its Fc region.
24. The method of any of embodiments 1-23, wherein the Fc region of the CD3-specific IgG4 antibody from which the CD3-binding arm is derived comprises S228P. L234A, L235A, F405L, and R409K substitutions in its Fc region.
25. The method of any of embodiments 1-24, wherein the GPRC5D×CD3 bispecific antibody comprises a first heavy chain (HC1) having the amino acid sequence of SEQ ID NO: 32, a first light chain (LC1) having the amino acid sequence of SEQ ID NO: 33, a second heavy chain (HC2) having the amino acid sequence of SEQ ID NO: 22 and a second light chain (LC2) having the amino acid sequence of SEQ ID NO: 23.
26. The method of any of embodiments 1-24, wherein the GPRC5D×CD3 bispecific antibody comprises a first heavy chain (HC1) having at least 90% identity to the amino acid sequence of SEQ ID NO: 32, a first light chain (LC1) having at least 90% identity to the amino acid sequence of SEQ ID NO: 33, a second heavy chain (HC2) having at least 90% identity to the amino acid sequence of SEQ ID NO: 22 and a second light chain (LC2) having at least 90% identity to the amino acid sequence of SEQ ID NO: 23.
27. The method of any of embodiments 1-24, wherein the GPRC5D×CD3 bispecific antibody comprises a first heavy chain (HC1) having at least 95% identity to the amino acid sequence of SEQ ID NO: 32, a first light chain (LC1) having at least 95% identity to the amino acid sequence of SEQ ID NO: 33, a second heavy chain (HC2) having at least 95% identity to the amino acid sequence of SEQ ID NO: 22 and a second light chain (LC2) having at least 95% identity to the amino acid sequence of SEQ ID NO: 23.
28. The method of any of embodiments 1-24, wherein the GPRC5D×CD3 bispecific antibody comprises a first heavy chain (HC1) having at least 98% identity to the amino acid sequence of SEQ ID NO: 32, a first light chain (LC1) having at least 98% identity to the amino acid sequence of SEQ ID NO: 33, a second heavy chain (HC2) having at least 98% identity to the amino acid sequence of SEQ ID NO: 22 and a second light chain (LC2) having at least 98% identity to the amino acid sequence of SEQ ID NO: 23.
29. The method of any of embodiments 1-25, wherein the GPRC5D×CD3 bispecific antibody is talquetamab.
30. The method of any of embodiments 1-29, wherein the subject has relapsed or refractory multiple myeloma (e.g., the subject has had been treated with from 1 to 11 or from 1-10 prior lines of therapy, and/or the subject has previously received an autologous stem cell transplant).
31. The method of any of embodiments 1-30, wherein the subject has received at least three prior lines of therapy.
32. The method of any of embodiments 1-30, wherein the subject has received at least four prior lines of therapy.
33. The method of any of embodiments 1-30, wherein the subject has received at least five prior lines of therapy (penta-drug exposed).
34. The method of any of embodiments 1-30, wherein the subject has received at least three prior lines of therapy, including a proteasome inhibitor, an immunomodulatory agent and an anti-CD38 monoclonal antibody.
35. The method of any of embodiments 1-30, wherein the subject has received at least four prior lines of therapy, including a proteasome inhibitor, an immunomodulatory agent and an anti-CD38 monoclonal antibody.
36. The method of any of embodiments 1-35, wherein the subject has extramedullary disease (EMD).
37. The method of any of embodiments 1-36 comprising subcutaneously administering to the subject one or more step-up doses of the BCMA×CD3 bispecific antibody prior to administering a treatment dose of the BCMA×CD3 bispecific antibody.
38. The method of any of embodiments 1-37 comprising subcutaneously administering to the subject a treatment dose of the BCMA×CD3 bispecific antibody weekly (QW).
39. The method of any of embodiments 1-37 comprising subcutaneously administering to the subject a treatment dose of the BCMA×CD3 bispecific antibody every two weeks (Q2W).
40. The method of any of embodiments 1-37 comprising subcutaneously administering the BCMA×CD3 bispecific antibody weekly (QW) at a treatment dose of from about 750 μg/kg to about 1500 μg/kg.
41. The method of any of embodiments 1-37 comprising subcutaneously administering the BCMA×CD3 bispecific antibody weekly (QW) at a treatment dose of about 750 μg/kg.
42. The method of any of embodiments 1-37 comprising subcutaneously administering the BCMA×CD3 bispecific antibody weekly (QW) at a treatment dose of about 1500 μg/kg.
43. The method of any of embodiments 1-37 or 39 comprising subcutaneously administering the BCMA×CD3 bispecific antibody every two weeks (Q2W) at a treatment dose of from about 1500 μg/kg to about 3000 μg/kg.
44. The method of any of embodiments 1-37, 39 or 43 comprising subcutaneously administering the BCMA×CD3 bispecific antibody every two weeks (Q2W) at a treatment dose of about 1500 μg/kg.
45. The method of any of embodiments 1-37, 39 or 43 comprising subcutaneously administering the BCMA×CD3 bispecific antibody every two weeks (Q2W) at a treatment dose of about 3000 μg/kg.
46. The method of any of embodiments 1-45 comprising subcutaneously administering 2 or 3 step-up doses of the BCMA×CD3 bispecific antibody prior to subcutaneously administering a treatment dose.
47. The method of any of embodiments 1-46 comprising subcutaneously administering step-up doses of 60 μg/kg and 300 μg/kg of the BCMA×CD3 bispecific antibody prior to subcutaneously administering a treatment dose.
48. The method of any of embodiments 1-47 comprising subcutaneously administering step-up doses of 60 μg/kg and 300 μg/kg and 1500 μg/kg of the BCMA×CD3 bispecific antibody prior to subcutaneously administering a treatment dose.
49. The method of any of embodiments 1-48 comprising subcutaneously administering step-up doses of the BCMA×CD3 bispecific antibody 2-4 days apart from each other.
50. The method of any of embodiments 1-49 comprising subcutaneously administering to the subject one or more step-up doses of the GPRC5D×CD3 bispecific antibody prior to administering a treatment dose of the GPRC5D×CD3 bispecific antibody.
51. The method of any of embodiments 1-50 comprising subcutaneously administering to the subject a treatment dose of the GPRC5D×CD3 bispecific antibody weekly (QW).
52. The method of any of embodiments 1-50 comprising subcutaneously administering to the subject a treatment dose of the GPRC5D×CD3 bispecific antibody every two weeks (Q2W).
53. The method of any of embodiments 1-50 comprising subcutaneously administering the GPRC5D×CD3 bispecific antibody weekly (QW) at a treatment dose of from about 200 μg/kg to about 400 μg/kg.
54. The method of any of embodiments 1-50 comprising subcutaneously administering the GPRC5D×CD3 bispecific antibody weekly (QW) at a treatment dose of about 200 μg/kg.
55. The method of any of embodiments 1-50 comprising subcutaneously administering the GPRC5D×CD3 bispecific antibody weekly (QW) at a treatment dose of about 400 μg/kg.
56. The method of any of embodiments 1-50 comprising subcutaneously administering the GPRC5D×CD3 bispecific antibody every two weeks (Q2W) at a treatment dose of about 800 μg/kg.
57. The method of any of embodiments 1-56 comprising subcutaneously administering 2 or 3 step-up doses of the GPRC5D×CD3 bispecific antibody prior to subcutaneously administering a treatment dose.
58. The method of any of embodiments 1-57 comprising subcutaneously administering step-up doses of 10 μg/kg and 60 μg/kg of the GPRC5D×CD3 bispecific antibody prior to subcutaneously administering a treatment dose.
59. The method of any of embodiments 1-58 comprising subcutaneously administering step-up doses of 10 μg/kg and 60 μg/kg and 400 μg/kg of the GPRC5D×CD3 bispecific antibody prior to subcutaneously administering a treatment dose.
60. The method of any of embodiments 1-59 comprising subcutaneously administering step-up doses of the GPRC5D×CD3 bispecific antibody 2-4 days apart from each other.
61. The method of any of embodiments 1-36 comprising subcutaneously administering the BCMA×CD3 bispecific antibody every two weeks (Q2W) at a treatment dose of 3000 μg/kg and subcutaneously administering the GPRC5D×CD3 bispecific antibody every two weeks (Q2W) at a treatment dose of 80 μg/kg.
62. The method of any of embodiments 1-36 comprising subcutaneously administering the BCMA×CD3 bispecific antibody every two weeks (Q2W) at a treatment dose of 1500 μg/kg and subcutaneously administering the GPRC5D×CD3 bispecific antibody every two weeks (Q2W) at a treatment dose of 800 μg/kg.
63. The method of any of embodiments 1-36 comprising subcutaneously administering the BCMA×CD3 bispecific antibody weekly (QW) at a treatment dose of 1500 μg/kg and subcutaneously administering the GPRC5D×CD3 bispecific antibody weekly (QW) at a treatment dose of 800 μg/kg.
64. The method of any of embodiments 1-36 comprising subcutaneously administering the BCMA×CD3 bispecific antibody weekly (QW) at a treatment dose of 1500 μg/kg and subcutaneously administering the GPRC5D×CD3 bispecific antibody weekly (QW) at a treatment dose of 400 μg/kg.
65. The method of any of embodiments 1-36 comprising subcutaneously administering the BCMA×CD3 bispecific antibody weekly (QW) at a treatment dose of 1500 μg/kg and subcutaneously administering the GPRC5D×CD3 bispecific antibody weekly (QW) at a treatment dose of 200 μg/kg.
66. The method of any of embodiments 1-36 comprising subcutaneously administering the BCMA×CD3 bispecific antibody weekly (QW) at a treatment dose of 750 μg/kg and subcutaneously administering the GPRC5D×CD3 bispecific antibody weekly (QW) at a treatment dose of 200 μg/kg.
67. The method of any of embodiments 61-66 comprising subcutaneously administering one or more step-up doses of the BCMA×CD3 bispecific antibody prior to subcutaneously administering a treatment dose; and subcutaneously administering one or more step-up doses of the GPRC5D×CD3 bispecific antibody prior to subcutaneously administering a treatment dose.
68. The method of any of embodiments 61-67 comprising subcutaneously administering 2 or 3 step-up doses of the BCMA×CD3 bispecific antibody prior to subcutaneously administering a treatment dose; and subcutaneously administering 2 or 3 step-up doses of the GPRC5D×CD3 bispecific antibody prior to subcutaneously administering a treatment dose.
69. The method of any of embodiments 61-68 comprising subcutaneously administering step-up doses of 60 μg/kg and 300 μg/kg of the BCMA×CD3 bispecific antibody prior to subcutaneously administering a treatment dose; and subcutaneously administering step-up doses of 10 μg/kg and 60 μg/kg of the GPRC5D×CD3 bispecific antibody prior to subcutaneously administering a treatment dose.
70. The method of any of embodiments 61-69 comprising subcutaneously administering step-up doses of 60 μg/kg and 300 μg/kg and 1500 μg/kg of the BCMA×CD3 bispecific antibody prior to subcutaneously administering a treatment dose; and subcutaneously administering step-up doses of 10 μg/kg and 60 μg/kg and 300 μg/kg of the GPRC5D×CD3 bispecific antibody prior to subcutaneously administering a treatment dose.
71. The method of any of embodiments 61-69 comprising subcutaneously administering step-up doses of 60 μg/kg and 300 μg/kg and 1500 μg/kg of the BCMA×CD3 bispecific antibody prior to subcutaneously administering a treatment dose; and subcutaneously administering step-up doses of 10 μg/kg and 60 μg/kg and 400 μg/kg of the GPRC5D×CD3 bispecific antibody prior to subcutaneously administering a treatment dose.
72. The method of any of embodiments 67-71, wherein the step-up doses of the BCMA×CD3 bispecific antibody are administered 2-4 days apart from each other, and the step-up doses of the GPRC5D×CD3 bispecific antibody are administered 2-4 days apart from each other.
73. The method of any of embodiments 1-72, wherein each treatment dose of the BCMA×CD3 bispecific antibody is administered on the same day as each treatment dose of the GPRC5D×CD3 bispecific antibody.
74. The method of any of embodiments 1-73, wherein each step-up dose of the BCMA×CD3 bispecific antibody is administered on the same day as each step-up dose of the GPRC5D×CD3 bispecific antibody.
75. The method of any of embodiments 1-36, wherein a first treatment dose of the BCMA×CD3 bispecific antibody and a first treatment dose of the GPRC5D×CD3 bispecific antibody are administered subcutaneously on Cycle 1 Day 1 of a 28-day cycle, wherein:
subsequent treatment doses of the GPRC5D×CD3 bispecific antibody are administered Q2W at a dose amount of 800 μg/kg subcutaneously, e.g., on Days 1 and 15 (±3 days) of each 28-day cycle, and
subsequent treatment doses of the BCMA×CD3 bispecific antibody are administered Q2W at a dose amount of 3000 μg/kg subcutaneously, e.g., on Days 1 and 15 (±3 days) of a 28-day cycle.
76. The method of embodiment 75 comprising subcutaneously administering step-up doses of 60 μg/kg and 300 μg/kg and 1500 μg/kg of the BCMA×CD3 bispecific antibody prior to subcutaneously administering said first treatment dose; and subcutaneously administering step-up doses of 10 μg/kg and 60 μg/kg and 400 μg/kg of the GPRC5D×CD3 bispecific antibody prior to subcutaneously administering said first treatment dose, wherein said first treatment doses are administered 2 to 4 days after administration of the last step-up dose of each bispecific antibody.
76.5. The method of any of embodiments 1-76 comprising subcutaneously administering a first step-up dose of 60 μg/kg of the BCMA×CD3 bispecific antibody, then 2-4 days later subcutaneously administering a second step-up dose of 300 μg/kg of the BCMA×CD3 bispecific antibody, then 2-4 days later subcutaneously administering a third step-up dose of 1500 μg/kg of the BCMA×CD3 bispecific antibody, then 2-4 days later subcutaneously administering a first treatment dose of 3000 μg/kg of the BCMA×CD3 bispecific antibody; and
subcutaneously administering a first step-up dose of 10 μg/kg of the GPRC5D×CD3 bispecific antibody, then 2-4 days later subcutaneously administering a second step-up dose of 60 μg/kg of the GPRC5D×CD3 bispecific antibody, then 2-4 days later subcutaneously administering a third step-up dose of 300 μg/kg or 400 μg/kg of the GPRC5D×CD3 bispecific antibody, then 2-4 days later subcutaneously administering a first treatment dose of 800 μg/kg of the GPRC5D×CD3 bispecific antibody, and
thereafter, subcutaneously administering the BCMA×CD3 bispecific antibody every two weeks (Q2W) at a treatment dose of 3000 μg/kg and subcutaneously administering the GPRC5D×CD3 bispecific antibody every two weeks (Q2W) at a treatment dose of 800 μg/kg.
77. The method of any of embodiments 1-76.5, wherein the method achieves a partial response, a very good partial response, a complete response or a stringent complete response in the subject, as determined by IMWG response criteria.
78. The method of embodiment 77, wherein the subject has extramedullary disease (EMD).
79. The method of any of embodiments 1-77, wherein the method achieves an overall response rate of at least 70% in a population of subjects with relapsed or refractory multiple myeloma (RRMM).
80. The method of embodiment 79, wherein the subjects have extramedullary disease (EMD).
81. The method of any of embodiments 1-77, wherein the method achieves an overall response rate of at least 80% in a population of subjects with RRMM.
82. The method of embodiment 81, wherein the subjects have extramedullary disease (EMD).
83. The method of any of embodiments 1-77, wherein the method achieves an overall response rate of at least 85% in a population of subjects with RRMM.
84. The method of embodiment 83, wherein the subjects have extramedullary disease (EMD).
85. The method of any of embodiments 1-77, wherein the method achieves an overall response rate of at least 90% in a population of subjects with RRMM.
86. The method of any of embodiments 1-77, wherein the method achieves an overall response rate of at least 95% in a population of subjects with RRMM.
87. The method of any of embodiments 1-77, wherein the method achieves a complete response or a stringent complete response rate of at least 20% in a population of subjects with RRMM.
88. The method of embodiment 87, wherein the subjects have extramedullary disease (EMD).
89. The method of any of embodiments 1-77, wherein the method achieves a complete response or a stringent complete response rate of at least 25% in a population of subjects with RRMM.
90. The method of embodiment 89, wherein the subjects have extramedullary disease (EMD).
91. The method of any of embodiments 1-77, wherein the method achieves a complete response or a stringent complete response rate of at least 30% in a population of subjects with RRMM.
92. The method of embodiment 91, wherein the subjects have extramedullary disease (EMD).
93. The method of any of embodiments 1-77, wherein the method achieves a complete response or a stringent complete response rate of at least 35% in a population of subjects with RRMM.
94. The method of embodiment 93, wherein the subjects have extramedullary disease (EMD).
95. The method of any of embodiments 1-77, wherein the method achieves a complete response or a stringent complete response rate of at least 40% in a population of subjects with RRMM.
96. The method of embodiment 95, wherein the subjects have extramedullary disease (EMD).
97. A combination dosing regimen for the treatment of multiple myeloma comprises a BCMA×CD3 bispecitic antibody and a GPRC5D×CD3 bispecific antibody, wherein said bispecific antibodies may be selected from any of embodiments 2-29.
98. The method of any of embodiments 1-77, wherein the subject has received between 1-4 prior lines of therapy including an anti-CD38 antibody (e.g., daratumumab) and lenalidomide, and wherein the method comprises:
treating the subject according to a therapeutically effective regimen that comprises sequential 28-day treatment cycles, wherein:
one or more step-up doses of the GPRC5D×CD3 bispecific antibody (e.g., talquetamab) are subcutaneously administered to the subject during a step-up phase in Cycle 1, and then each treatment dose of the GPRC5D×CD3 bispecific antibody is subcutaneously administered to the subject in an amount of 0.8 mg/kg,
wherein the treatment doses of the GPRC5D×CD3 bispecific antibody are administered to the subject on a bi-weekly dosing schedule (Q2W) starting from treatment Cycle 2 (e.g., on Days 1 and 15), and then treatment doses of the GPRC5D×CD3 bispecific antibody are subcutaneously administered to the subject on a monthly dosing schedule (Q4W) either: (i) starting at treatment Cycle 5 if the subject has achieved a very good partial response, a complete response or a stringent complete response, as determined by IMWG response criteria; or (ii) starting at treatment Cycle 7, regardless of clinical response in the subject.
99. The method of embodiment 98, wherein one or more step-up doses of the BCMA×CD3 bispecific antibody (e.g., teclistamab) are subcutaneously administered to the subject during a step-up phase in Cycle 1, and each treatment dose of the BCMA×CD3 bispecific antibody is subcutaneously administered to the subject in an amount of 3.0 mg/kg,
wherein the treatment doses of the BCMA×CD3 bispecific antibody are administered to the subject on a bi-weekly dosing schedule (Q2W) starting from treatment Cycle 2 (e.g., on Days 1 and 15), and then treatment doses of the BCMA×CD3 bispecific antibody are subcutaneously administered to the subject on a monthly dosing schedule (Q4W) either: (i) starting at treatment Cycle 3 if the subject has achieved a partial response, a very good partial response, a complete response or a stringent complete response, as determined by IMWG response criteria; or (ii) starting at treatment Cycle 7, regardless of clinical response in the subject.
100. A method of improving median progression free survival (PFS) in a population of subjects with relapsed or refractory multiple myeloma that have received between 1-4 prior lines of therapy including an anti-CD38 antibody (e.g., daratumumab) and lenalidomide, said method comprising administering to the population of subjects a combination therapy comprising a therapeutically effective amount of a GPRC5D×CD3 bispecific antibody (e.g., talquetamab) and a therapeutically effective amount of a BCMA×CD3 bispecific antibody (e.g., teclistamab), wherein the method comprises:
treating the subject according to a therapeutically effective regimen that comprises sequential 28-day treatment cycles, wherein:
one or more step-up doses of the GPRC5D×CD3 bispecific antibody are subcutaneously administered to the subject during a step-up phase in Cycle 1, and then each treatment dose of the GPRC5D×CD3 bispecific antibody is subcutaneously administered to the subject in an amount of 0.8 mg/kg,
wherein the treatment doses of the GPRC5D×CD3 bispecific antibody are administered to the subject on a bi-weekly dosing schedule (Q2W) starting from treatment Cycle 2 (e.g., on Days 1 and 15), and then treatment doses of the GPRC5D×CD3 bispecific antibody are subcutaneously administered to the subject on a monthly dosing schedule (Q4W) either: (i) starting at treatment Cycle 5 if the subject has achieved a very good partial response, a complete response or a stringent complete response, as determined by IMWG response criteria; or (ii) starting at treatment Cycle 7, regardless of clinical response in the subject, and
wherein one or more step-up doses of the BCMA×CD3 bispecific antibody (e.g., teclistamab) are subcutaneously administered to the subject during a step-up phase in Cycle 1, and each treatment dose of the BCMA×CD3 bispecific antibody is subcutaneously administered to the subject in an amount of 3.0 mg/kg,
wherein the treatment doses of the BCMA×CD3 bispecific antibody are administered to the subject on a bi-weekly dosing schedule (Q2W) starting from treatment Cycle 2 (e.g., on Days 1 and 15), and then treatment doses of the BCMA×CD3 bispecific antibody are subcutaneously administered to the subject on a monthly dosing schedule (Q4W) either: (i) starting at treatment Cycle 3 if the subject has achieved a partial response, a very good partial response, a complete response or a stringent complete response, as determined by IMWG response criteria, or (ii) starting at treatment Cycle 7, regardless of clinical response in the subject,
wherein the improvement in median PFS is relative to median PFS of a reference population of subjects with relapsed or refractory multiple myeloma that have received between 1-4 prior lines of therapy including an anti-CD38 antibody (e.g., daratumumab) and lenalidomide, said reference population having been administered either (i) elotuzumab, pomalidomide, and dexamethasone (EPd) or (ii) pomalidomide, bortezomib, and dexamethasone (PVd).
101. A method of improving overall response rate (ORR) in a population of subjects with relapsed or refractory multiple myeloma that have received between 1-4 prior lines of therapy including an anti-CD38 antibody (e.g., daratumumab) and lenalidomide, said method comprising administering to the population of subjects a combination therapy comprising a therapeutically effective amount of a GPRC5D×CD3 bispecific antibody (e.g., talquetamab) and a therapeutically effective amount of a BCMA×CD3 bispecific antibody (e.g., teclistamab), wherein the method comprises:
treating the subjects according to a therapeutically effective regimen that comprises sequential 28-day treatment cycles, wherein:
one or more step-up doses of the GPRC5D×CD3 bispecific antibody are subcutaneously administered to the subject during a step-up phase in Cycle 1, and then each treatment dose of the GPRC5D×CD3 bispecific antibody is subcutaneously administered to the subject in an amount of 0.8 mg/kg,
wherein the treatment doses of the GPRC5D×CD3 bispecific antibody are administered to the subject on a bi-weekly dosing schedule (Q2W) starting from treatment Cycle 2 (e.g., on Days 1 and 15), and then treatment doses of the GPRC5D×CD3 bispecific antibody are subcutaneously administered to the subject on a monthly dosing schedule (Q4W) either: (i) starting at treatment Cycle 5 if the subject has achieved a very good partial response, a complete response or a stringent complete response, as determined by IMWG response criteria; or (ii) starting at treatment Cycle 7, regardless of clinical response in the subject, and
wherein one or more step-up doses of the BCMA×CD3 bispecific antibody (e.g., teclistamab) are subcutaneously administered to the subject during a step-up phase in Cycle 1, and each treatment dose of the BCMA×CD3 bispecific antibody is subcutaneously administered to the subject in an amount of 3.0 mg/kg,
wherein the treatment doses of the BCMA×CD3 bispecific antibody are administered to the subject on a bi-weekly dosing schedule (Q2W) starting from treatment Cycle 2 (e.g., on Days 1 and 15), and then treatment doses of the BCMA×CD3 bispecific antibody are subcutaneously administered to the subject on a monthly dosing schedule (Q4W) either: (i) starting at treatment Cycle 3 if the subject has achieved a partial response, a very good partial response, a complete response or a stringent complete response, as determined by IMWG response criteria; or (ii) starting at treatment Cycle 7, regardless of clinical response in the subject,
wherein the improvement in ORR is relative to ORR of a reference population of subjects with relapsed or refractory multiple myeloma that have received between 1-4 prior lines of therapy including an anti-CD38 antibody (e.g., daratumumab) and lenalidomide, said reference population having been administered either (i) elotuzumab, pomalidomide, and dexamethasone (EPd) or (ii) pomalidomide, bortezomib, and dexamethasone (PVd).102. The method of any of embodiments 98-101, wherein the step-up phase comprises subcutaneously administering step-up doses of 0.01 mg/kg and 0.06 mg/kg of the GPRC5D×CD3 bispecific antibody 2-4 days apart from each other (e.g., on Days 1 and 4, respectively).
103. The method of any of embodiments 98-101, wherein the step-up phase comprises subcutaneously administering step-up doses of 0.01 mg/kg, 0.06 mg/kg and 0.4 mg/kg of the GPRC5D×CD3 bispecific antibody 2-4 days apart from each other (e.g., on Days 1, 4 and 8, respectively).
104. The method of any of embodiments 98-101, wherein the step-up phase comprises subcutaneously administering step-up doses of 0.01 mg/kg, 0.06 mg/kg and 0.4 mg/kg of the GPRC5D×CD3 bispecific antibody 2-4 days apart from each other (e.g., on Days 1, 4 and 8, respectively) and then subcutaneously administering the treatment dose of 0.8 mg/kg of the GPRC5D×CD3 bispecific antibody at least two days after the 0.4 mg/kg dose (e.g., between Days 7-15, such as Day 15).
105. The method of any of embodiments 98-104, wherein the step-up phase comprises subcutaneously administering step-up doses of 0.06 mg/kg and 0.3 mg/kg of the BCMA×CD3 bispecific antibody 2-4 days apart from each other (e.g., on Days 1 and 4, respectively).
106. The method of any of embodiments 98-104, wherein the step-up phase comprises subcutaneously administering step-up doses of 0.06 mg/kg, 0.3 mg/kg and 1.5 mg/kg of the BCMA×CD3 bispecific antibody 2-4 days apart from each other (e.g., on Days 1, 4 and 8, respectively).
107. The method of any of embodiments 98-104, wherein the step-up phase comprises subcutaneously administering step-up doses of 0.06 mg/kg, 0.3 mg/kg and 1.5 mg/kg of the BCMA×CD3 bispecific antibody 2-4 days apart from each other (e.g., on Days 1, 4 and 8, respectively) and then subcutaneously administering a treatment dose of 3.0 mg/kg of the BCMA×CD3 bispecific antibody at least two days after the 1.5 mg/kg dose (e.g., between Days 7-15, such as Day 15).
108. The method of any of embodiments 1-36, wherein the subject has received between 1-4 prior lines of therapy including an anti-CD38 antibody (e.g., daratumumab) and lenalidomide, and wherein the method comprises:
treating the subject according to a therapeutically effective regimen that comprises sequential 28-day, treatment cycles, wherein:
step-up doses of the GPRC5D×CD3 bispecific antibody (e.g., talquetamab) and the BCMA×CD3 bispecific antibody (e.g., teclistamab) are subcutaneously administered to the subject during a step-up phase in Cycle 1, and then:
treatment doses of the BCMA×CD3 bispecific antibody are subcutaneously administered to the subject, each in an amount of 3.0 mg/kg, on a monthly dosing schedule (Q4W) starting from treatment Cycle 2, regardless of clinical response in the subject; and
treatment doses of the GPRC5D×CD3 bispecific antibody are subcutaneously administered to the subject, each in an amount of 0.8 mg/on a bi-weekly dosing schedule (Q2W) starting from the treatment Cycle 2 (e.g., on Days 1 and 15), and then treatment doses of the GPRC5D×CD3 bispecific antibody are subcutaneously administered to the subject on a monthly dosing schedule (Q4W) either: (i) starting at treatment Cycle 5 if the subject has achieved a very good partial response, a complete response or a stringent complete response, as determined by IMWG response criteria; or (ii) starting at treatment Cycle 7 if the subject has achieved a partial response, a very good partial response, a complete response or a stringent complete response, as determined by IMWG response criteria; or (iii) after treatment Cycle 7 when a partial response, a very good partial response, a complete response or a stringent complete response is achieved, as determined by IMWG response criteria.
109. The method of claim 108, wherein the GPRC5D×CD3 bispecific antibody (e.g., talquetamab) and the BCMA×CD3 bispecific antibody (e.g., teclistamab) are administered at least 15 minutes apart from each other each day that doses are administered.
110. A method of improving median progression free survival (PFS) in a population of subjects with relapsed or refractory multiple myeloma that have received between 1-4 prior lines of therapy including an anti-CD38 antibody (e.g., daratumumab) and lenalidomide, said method comprising administering to the population of subjects a combination therapy comprising a therapeutically effective amount of a GPRC5D×CD3 bispecific antibody (e.g., talquetamab) and a therapeutically effective amount of a BCMA×CD3 bispecific antibody (e.g., teclistamab), wherein the method comprises:
treating the subject according to a therapeutically effective regimen that comprises sequential 28-day, treatment cycles, wherein:
step-up doses of the GPRC5D×CD3 bispecific antibody (e.g., talquetamab) and the BCMA×CD3 bispecific antibody (e.g., teclistamab) are subcutaneously administered to the subject during a step-up phase in Cycle 1, and then
treatment doses of the BCMA×CD3 bispecific antibody are subcutaneously administered to the subject, each in an amount of 3.0 mg/kg, on a monthly dosing schedule (Q4W) starting from treatment Cycle 2, regardless of clinical response in the subject; and
treatment doses of the GPRC5D×CD3 bispecific antibody are subcutaneously administered to the subject, each in an amount of 0.8 mg/on a bi-weekly dosing schedule (Q2W) starting from treatment Cycle 2 (e.g., on Days 1 and 15), and then treatment doses of the GPRC5D×CD3 bispecific antibody are subcutaneously administered to the subject on a monthly dosing schedule (Q4W) either: (i) starting at treatment Cycle 5 if the subject has achieved a very good partial response, a complete response or a stringent complete response, as determined by IMWG response criteria; or (ii) starting at treatment Cycle 7 if the subject has achieved a partial response, a very good partial response, a complete response or a stringent complete response, as determined by IMWG response criteria; or (iii) after treatment Cycle 7 when a partial response, a very good partial response, a complete response or a stringent complete response is achieved, as determined by IMWG response criteria,
wherein the improvement in median PFS is relative to median PFS of a reference population of subjects with relapsed or refractory multiple myeloma that have received between 1-4 prior lines of therapy including an anti-CD38 antibody (e.g., daratumumab) and lenalidomide, said reference population having been administered either (i) elotuzumab, pomalidomide, and dexamethasone (EPd) or (ii) pomalidomide, bortezomib, and dexamethasone (PVd).
111. A method of improving overall response rate (ORR) in a population of subjects with relapsed or refractory multiple myeloma that have received between 1-4 prior lines of therapy including an anti-CD38 antibody (e.g., daratumumab) and lenalidomide, said method comprising administering to the population of subjects a combination therapy comprising a therapeutically effective amount of a GPRC5D×CD3 bispecific antibody (e.g., talquetamab) and a therapeutically effective amount of a BCMA×CD3 bispecific antibody (e.g., teclistamab), wherein the method comprises:
treating the subjects according to a therapeutically effective regimen that comprises sequential 28-day treatment cycles, wherein:
step-up doses of the GPRC5D×CD3 bispecific antibody (e.g., talquetamab) and the BCMA×CD3 bispecific antibody (e.g., teclistamab) are subcutaneously administered to the subject during a step-up phase in Cycle 1, and then
treatment doses of the BCMA×CD3 bispecific antibody are subcutaneously administered to the subject, each in an amount of 3.0 mg/kg, on a monthly dosing schedule (Q4W) starting from treatment Cycle 2, regardless of clinical response in the subject; and
treatment doses of the GPRC5D×CD3 bispecific antibody are subcutaneously administered to the subject, each in an amount of 0.8 mg/on a bi-weekly dosing schedule (Q2W) starting from treatment Cycle 2 (e.g., on Days 1 and 15), and then treatment doses of the GPRC5D×CD3 bispecific antibody are subcutaneously administered to the subject on a monthly dosing schedule (Q4W) either: (i) starting at treatment Cycle 5 if the subject has achieved a very good partial response, a complete response or a stringent complete response, as determined by IMWG response criteria; or (ii) starting at treatment Cycle 7 if the subject has achieved a partial response, a very good partial response, a complete response or a stringent complete response, as determined by IMWG response criteria; or (iii) after treatment Cycle 7 when a partial response, a very good partial response, a complete response or a stringent complete response is achieved, as determined by IMWG response criteria,
wherein the improvement in ORR is relative to ORR of a reference population of subjects with relapsed or refractory multiple myeloma that have received between 1-4 prior lines of therapy including an anti-CD38 antibody (e.g., daratumumab) and lenalidomide, said reference population having been administered either (i) elotuzumab, pomalidomide, and dexamethasone (EPd) or (ii) pomalidomide, bortezomib, and dexamethasone (PVd).
112. The method of any of embodiments 108-111, wherein the step-up phase comprises subcutaneously administering step-up doses of 0.01 mg/kg and 0.06 mg/kg of the GPRC5D×CD3 bispecific antibody 2-4 days apart from each other (e.g., on Days 1 and 4, respectively).
113. The method of any of embodiments 108-111, wherein the step-up phase comprises subcutaneously administering step-up doses of 0.01 mg/kg, 0.06 mg/kg and 0.4 mg/kg of the GPRC5D×CD3 bispecific antibody 2-4 days apart from each other (e.g., on Days 1, 4 and 8, respectively).
114. The method of any of embodiments 108-111, wherein the step-up phase comprises subcutaneously administering step-up doses of 0.01 mg/kg, 0.06 mg/kg and 0.4 mg/kg of the GPRC5D×CD3 bispecific antibody 2-4 days apart from each other (e.g., on Days 1, 4 and 8, respectively) and then subcutaneously administering the treatment dose of 0.8 mg/kg of the GPRC5D×CD3 bispecific antibody at least two days after the 0.4 mg/kg dose (e.g., between Days 7-15, such as Day 15).
115. The method of any of embodiments 108-114, wherein the step-up phase comprises subcutaneously administering step-up doses of 0.06 mg/kg and 0.3 mg/kg of the BCMA×CD3 bispecific antibody 2-4 days apart from each other (e.g., on Days 1 and 4, respectively).
116. The method of any of embodiments 108-114, wherein the step-up phase comprises subcutaneously administering step-up doses of 0.06 mg/kg, 0.3 mg/kg and 1.5 mg/kg of the BCMA×CD3 bispecific antibody 2-4 days apart from each other (e.g., on Days 1, 4 and 8, respectively).
117. The method of any of embodiments 108-114, wherein the step-up phase comprises subcutaneously administering step-up doses of 0.06 mg/kg, 0.3 mg/kg and 1.5 mg/kg of the BCMA×CD3 bispecific antibody 2-4 days apart from each other (e.g., on Days 1, 4 and 8, respectively) and then subcutaneously administering a dose of 1.5 mg/kg of the BCMA×CD3 bispecific antibody at least two days after the 1.5 mg/kg step-up dose (e.g., between Days 7-15, such as Day 15).
118. The method of any of embodiments 1-36, comprising:
subcutaneously administering to the subject step-up doses of the BCMA×CD3 bispecific antibody (e.g., teclistamab) and the GPRC5D×CD3 bispecific antibody (e.g., talquetamab), and then
subcutaneously administering treatment doses of 3000 μg/kg of the BCMA×CD3 bispecific antibody and 800 μg/kg of the GPRC5D×CD3 bispecific antibody biweekly (Q2W) for at least four 28-day cycles (e.g., on Days 1 and 15 (±3 days) of each cycle); and
(i) subcutaneously administering the treatment doses of 3000 μg/kg of the BCMA×CD3 bispecific antibody and 800 μg/kg of the GPRC5D×CD3 bispecific antibody monthly (Q4W) (e.g., on Day 1 of each 28-day cycle) when the subject has achieved a response of VGPR or better and received a minimum of four 28-day cycles of treatment (e.g., starting on Cycle 5); or
(ii) subcutaneously administering the treatment doses of 3000 μg/kg of the BCMA×CD3 bispecific antibody and 800 μg/kg of the GPRC5D×CD3 bispecific antibody monthly (Q4W) (e.g., on Day 1 of each 28-day cycle), regardless of clinical response in the subject, after the subject has completed 6 28-day cycles of treatment (e.g., starting on Cycle 7).
119. The method of embodiment 118, comprising administering 2 or 3 step-up doses of the BCMA×CD3 bispecific antibody, and 2 or 3 step-up doses of the GPRC5D×CD3 bispecific antibody, wherein step-up doses are administered 2-4 days apart from each other.
120. The method of any of embodiment 118, comprising administering 3 step-up doses of the BCMA×CD3 bispecific antibody (e.g., 60 μg/kg and 300 μg/kg and 1500 μg/kg) and 3 step-up doses of the GPRC5D×CD3 bispecific antibody (e.g., 10 μg/kg and 60 μg/kg and 400 μg/kg), wherein step-up doses are administered 2-4 days apart from each other.
121. The method of any of embodiments 118-120, comprising administering the first treatment doses of the BCMA×CD3 bispecific antibody and the GPRC5D×CD3 bispecific antibody 2 to 4 days after the last step-up doses (e.g., starting on Cycle 1, Day 1).
122. The method of any of embodiments 118-121, wherein the subject has relapsed or refractory multiple myeloma with extramedullary disease, and has previously received a proteasome inhibitor, an immunomodulatory agent and an anti-CD38 monoclonal antibody.
123. The method of any of embodiments 1-122, wherein the method achieves an overall response rate of at least 60% in a population of subjects with relapsed or refractory multiple myeloma (RRMM).
124. The method of embodiment 123, wherein the subjects have extramedullary disease (EMD).
125. The method of any of embodiments 1-122, wherein the method achieves an overall response rate of at least 65% in a population of subjects with relapsed or refractory multiple myeloma (RRMM).
126. The method of embodiment 125, wherein the subjects have extramedullary disease (EMD).
127. The method of any of embodiments 1-126, wherein each subject with EMD has one or more focus of EMD meeting the following criterion: any extramedullary plasmacytoma not contiguous with a bone lesion ≥2 cm (at its greatest dimension) diameter on PET-CT and not previously radiated.
128. The method of any of embodiments 1-127, wherein the GPRC5D×CD3 bispecific antibody (e.g., talquetamab) and the BCMA×CD3 bispecific antibody (e.g., teclistamab) are administered at least 15 minutes apart from each other on each day that doses are administered.
129. The method of embodiment 128, wherein the GPRC5D×CD3 bispecific antibody (e.g., talquetamab) and the BCMA×CD3 bispecific antibody (e.g., teclistamab) are administered within about 30 (±10) minutes of each other.
130. The method of embodiment 128 or 129, wherein each dose of the BCMA×CD3 bispecific antibody is administered before each dose of the GPRC5D×CD3 bispecific antibody.
131. The method of embodiment 128 or 129, wherein each dose of the GPRC5D×CD3 bispecific antibody is administered before each dose of the BCMA×CD3 bispecific antibody.
132. The method of any of embodiments 1-131, wherein the combination regimen comprising the BCMA×CD3 bispecific antibody and the GPRC5D×CD3 bispecific antibody is administered to the subject for 26 treatment cycles.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

### EXAMPLES

The following examples are provided to further describe some of the embodiments disclosed herein. The examples are intended to illustrate, not to limit, the disclosed embodiments.

### Example 1: Results from the Phase 1b RedirecTT-1 Trial (NCT04586426) with Teclistamab (Tec) and Talquetamab (Tal) Simultaneously Targeting BCMA and GPRC5D in Patients with Relapsed/Refractory Multiple Myeloma (RRMM)

Study Name: RedirecTT-1: NCT No.: NCT04586426

### Antibodies

Anti-BCMA/anti-CD3 antibody teclistamab (also called Tec) (e.g., described in WO2017031104A1, the content of which is incorporated herein by reference in its entirety) was made by Janssen Pharmaceuticals. Teclistamab comprises a BCMA binding arm BCMB69 and a CD3 binding arm CD3219, the amino acid sequences of which are shown in Table 11 and Table 12, respectively.

Anti-GPRC5D/anti-CD3 antibody talquetamab (also called Tal) (e.g., described in U.S. Pat. No. 10,562,968, the content of which is incorporated herein by reference in its entirety) was made by Janssen Pharmaceuticals. It was produced by cultivation of recombinant Chinese Hamster Ovary cells followed by isolation, chromatographic purification, and formulation. Talquetamab comprises a GPRC5D binding arm GC5B596 and a CD3 binding arm CD3B3219, the amino acid sequences of which are shown in Table 13 and Table 12, respectively.

TABLE 11
Sequences of BCMA binding arm of Teclistamab

| | | | SEQ |
|---|---|---|---|
| | | | ID |
| | Region | Sequence | NO: |
| BCMB69 | HCDR1 | SGSYFWG | 4 |
| | HCDR2 | SIYYSGITYYNPSLKS | 5 |
| | HCDR3 | HDGAVAGLEDY | 6 |
| | LCDR1 | GGNNIGSKSVH | 7 |
| | LCDR2 | DDSDRPS | 8 |
| | LCDR3 | QVWDSSSDHVV | 9 |
| | VH | QLQLQESGPGLVKPSETLSLTCTVSGGSISSG | 10 |
| | | SYFWGWIRQPPGKGLEWIGSIYYSGITYYNPS | |
| | | LKSRVTISVDTSKNQFSLKLSSVTAADTAVYY | |
| | | CARHDGAVAGLFDYWGQGTLVTVSS | |
| | VL | SYVLTQPPSVSVAPGQTARITCGGNNIGSKSVH | 11 |
| | | WYQQPPGQAPVVVVYDDSDRPSGIPERFSGSN | |
| | | SGNTATLTISRVEAGDEAVYYCQVWDSSSDHV | |
| | | VFGGGTKLTVL | |
| | HC | QLQLQESGPGLVKPSETLSLTCTVSGGSISSG | 12 |
| | | SYFWGWIRQPPGKGLEWIGSIYYSGITYYNPSL | |
| | | KSRVTISVDTSKNQFSLKLSSVTAADTAVYYCA | |
| | | RHDGAVAGLFDYWGQGTLVTVSSASTKGPSVFP | |
| | | LAPCSRSTSESTAALGCLVKDYFPEPVTVSWNS | |
| | | GALTSGVHTFPAVLQSSGLYSLSSVVTVPSSSL | |
| | | GTKTYTCNVDHKPSNTKVDKRVESKYGPPCPPC | |
| | | PAPEAAGGPSVFLFPPKPKDTLMISRTPEVTCV | |
| | | VVDVSQEDPEVQFNWYVDGVEVHNAKTKPRE | |
| | | EQFNSTYRVVSVLTVLHQDWLNGKEYKCKVSN | |
| | | KGLPSSIEKTISKAKGQPREPQVYTLPPSQEEM | |
| | | TKNQVSLTCLVKGFYPSDIAVEWESNGQPENN | |
| | | YKTTPPVLDSDGSFFLYSRLTVDKSRWQEGNV | |
| | | FSCSVMHEALHNHYTQKSLSLSLGK | |
| | LC | SYVLTQPPSVSVAPGQTARITCGGNNIGSKSVH | 13 |
| | | WYQQPPGQAPVVVVYDDSDRPSGIPERFSGSN | |
| | | SGNTATLTISRVEAGDEAVYYCQVWDSSSDHV | |
| | | VFGGGTKLTVLGQPKAAPSVTLFPPSSEELQAN | |
| | | KATLVCLISDFYPGAVTVAWKGDSSPVKAGVE | |
| | | TTTPSKQSNNKYAASSYLSLTPEQWKSHRSYSC | |
| | | QVTHEGSTVEKTVAPTECS | |

TABLE 12
Sequences of CD3 binding arm of
Teclistamab & Talquetamab

| | | | SEQ |
|---|---|---|---|
| | | | ID |
| | Region | Sequence | NO: |
| CD3B219 | HCDR1 | TYAMN | 14 |
| | HCDR2 | RIRSKYNNYATYYAASVKG | 15 |
| | HCDR3 | HGNFGNSYVSWFAY | 16 |
| | LCDR1 | RSSTGAVTTSNYAN | 17 |
| | LCDR2 | GTNKRAP | 18 |
| | LCDR3 | ALWYSNLWV | 19 |
| | VH | EVQLVESGGGLVQPGGSLRLSCAASGFTENT | 20 |
| | | YAMNWVRQAPGKGLEWVARIRSKYNNYAT | |
| | | YYAASVKGRFTISRDDSKNSLYLQMNSLKTE | |
| | | DTAVYYCARHGNFGNSYVSWFAYWGQGTL | |
| | | VTVSS | |
| | VL | QTVVTQEPSLTVSPGGTVTLTCRSSTGAVTT | 21 |
| | | SNYANWVQQKPGQAPRGLIGGTNKRAPGTP | |
| | | ARFSGSLLGGKAALTLSGVQPEDEAEYYCAL | |
| | | WYSNLWVFGGGTKLTVLGQP | |
| | HC | EVQLVESGGGLVQPGGSLRLSCAASGFTENT | 22 |
| | | YAMNWVRQAPGKGLEWVARIRSKYNNYAT | |
| | | YYAASVKGRFTISRDDSKNSLYLQMNSLKTE | |
| | | DTAVYYCARHGNFGNSYVSWFAYWGQGTLV | |
| | | TVSSASTKGPSVFPLAPCSRSTSESTAALGC | |
| | | LVKDYFPEPVTVSWNSGALTSGVHTFPAVL | |
| | | QSSGLYSLSSVVTVPSSSLGTKTYTCNVDHK | |
| | | PSNTKVDKRVESKYGPPCPPCPAPEAAGGPS | |
| | | VFLFPPKPKDTLMISRTPEVTCVVVDVSQED | |
| | | PEVQFNWYVDGVEVHNAKTKPREEQFNSTY | |
| | | RVVSVLTVLHQDWLNGKEYKCKVSNKGLPSS | |
| | | IEKTISKAKGQPREPQVYTLPPSQEEMTKNQ | |
| | | VSLTCLVKGFYPSDIAVEWESNGQPENNYKT | |
| | | TPPVLDSDGSFLLYSKLTVDKSRWQEGNVFS | |
| | | CSVMHEALHNHYTQKSLSLSLGK | |
| | LC | QTVVTQEPSLTVSPGGTVTLTCRSSTGAVTT | 23 |
| | | SNYANWVQQKPGQAPRGLIGGTNKRAPGTP | |
| | | ARFSGSLLGGKAALTLSGVQPEDEAEYYCAL | |
| | | WYSNLWVFGGGTKLTVLGQPKAAPSVTLFP | |
| | | PSSEELQANKATLVCLISDFYPGAVTVAWKA | |
| | | DSSPVKAGVETTTPSKQSNNKYAASSYLSLT | |
| | | PEQWKSHRSYSCQVTHEGSTVEKTVAPTECS | |

TABLE 13
Sequences of GPRC5D binding arm of Talquetamab

| | | | SEQ ID |
|---|---|---|---|
| | Region | Sequence | NO: |
| GC5B596 | HCDR1 | GYTMN | 24 |
| | HCDR2 | LINPYNSDTNYAQKLQG | 25 |
| | HCDR3 | VALRVALDY | 26 |
| | LCDR1 | KASQNVATHVG | 27 |
| | LCDR2 | SASYRYS | 28 |
| | LCDR3 | QQYNRYPYT | 29 |
| | VH | QVQLVQSGAEVKKPGASVKVSCKASGYSF | 30 |
| | | TGYTMNWVRQAPGQGLEWMGLINPYNSD | |
| | | TNYAQKLQGRVTMTTDTSTSTAYMELRSL | |
| | | RSDDTAVYYCARVALRVALDYWGQGTLV | |
| | | TVSS | |
| | VL | DIQMTQSPSSLSASVGDRVTITCKASQNVA | 31 |
| | | THVGWYQQKPGKAPKRLIYSASYRYSGVP | |
| | | SRFSGSGSGTEFTLTISNLQPEDFATYYCQ | |
| | | QYNRYPYTFGQGTKLEIK | |
| | HC | QVQLVQSGAEVKKPGASVKVSCKASGYSF | 32 |
| | | TGYTMNWVRQAPGQGLEWMGLINPYNSD | |
| | | TNYAQKLQGRVTMTTDTSTSTAYMELRSL | |
| | | RSDDTAVYYCARVALRVALDYWGQGTLVT | |
| | | VSSASTKGPSVFPLAPCSRSTSESTAALGC | |
| | | LVKDYFPEPVTVSWNSGALTSGVHTFPAV | |
| | | LQSSGLYSLSSVVTVPSSSLGTKTYTCNVD | |
| | | HKPSNTKVDKRVESKYGPPCPPCPAPEAAG | |
| | | GPSVFLFPPKPKDTLMISRTPEVTCVVVDV | |
| | | SQEDPEVQFNWYVDGVEVHNAKTKPREE | |
| | | QFNSTYRVVSVLTVLHQDWLNGKEYKCKV | |
| | | SNKGLPSSIEKTISKAKGQPREPQVYTLPP | |
| | | SQEEMTKNQVSLTCLVKGFYPSDIAVEWE | |
| | | SNGQPENNYKTTPPVLDSDGSFFLYSRLTV | |
| | | DKSRWQEGNVFSCSVMHEALHNHYTQKS | |
| | | LSLSLGK | |
| | LC | DIQMTQSPSSLSASVGDRVTITCKASQNVA | 33 |
| | | THVGWYQQKPGKAPKRLIYSASYRYSGVPS | |
| | | RFSGSGSGTEFTLTISNLQPEDFATYYCQQ | |
| | | YNRYPYTFGQGTKLEIKRTVAAPSVFIFPP | |
| | | SDEQLKSGTASVVCLLNNFYPREAKVQWK | |
| | | VDNALQSGNSQESVTEQDSKDSTYSLSSTL | |
| | | TLSKADYEKHKVYACEVTHQGLSSPVTKS | |
| | | FNRGEC | |

### Methods: Part 1 and Part 2

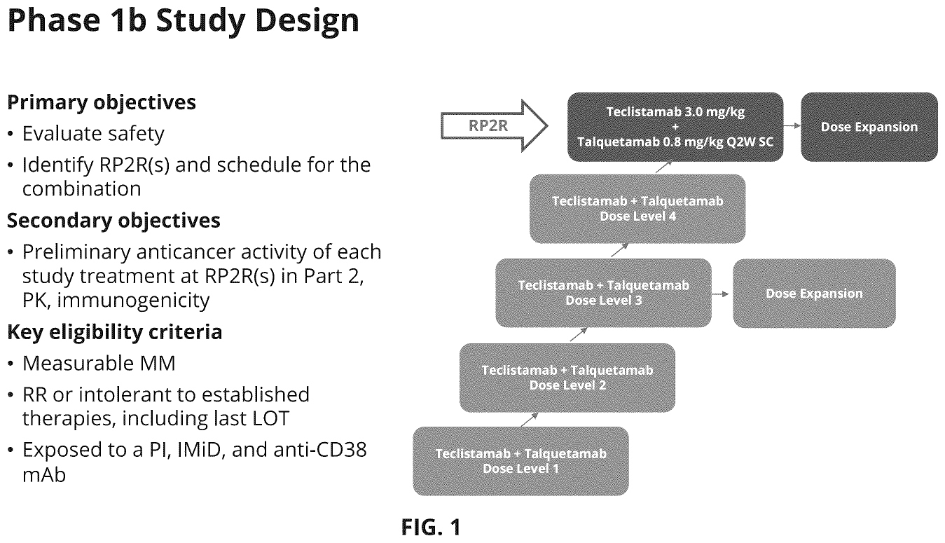

The purpose of this study is to identify the recommended Phase 2 regimen(s) (RP2R[s]) and schedule for the study treatment (Part 1) and to characterize the safety of the RP2R(s) for the study treatment (Part 2). The RP2R(s) will describe the combination doses and schedules of (tal+tec+dara) the treatment combinations to be pursued in Phase 2. An illustration of the study design is provided in FIG. 1.

Part 1 and Part 2: Escalation and expansion cohorts will examine the combination of talquetamab and teclistamab with or without SC daratumumab in participants with relapsed or refractory multiple myeloma.

Part 3: The Phase 2 component will examine the tal+tec combination in participants with EMD at the RP2R selected from Part 1 and Part 2.

Talquetamab is a humanized IgG4 PAA bispecific antibody designed to target G protein-coupled receptor family C group 5-member D (GPRC5D) and the CD3 molecule found on T lymphocytes (T cell). Teclistamab is a humanized IgG4 PAA bispecific antibody designed to target B cell maturation antigen (BCMA) and the CD3 molecule found on T cells. Daratumumab (also referred to as Dara) is a human IgG1κ monoclonal antibody that binds with high affinity to a unique epitope on cluster of differentiation 38 (CD38) in a variety of hematological malignancies including multiple myeloma.

This study includes 3 periods: screening phase (up to 28 days), treatment phase (start of study drug administration and continues until the completion of the end of treatment [EOT] visit); and a post-treatment follow-up phase (after end of treatment and up to 16 weeks after last dose of study drug(s) for each participant). End of study is defined as last study assessment for last participant in study. Total duration of study is up to 1 year and 6 months. Efficacy, safety, pharmacokinetics (PK), immunogenicity, and biomarkers are assessed at specified time points during this study. Participants safety and study conduct are monitored throughout the study.

### Dosing Regimens for Parts 1 and 2:

During dose escalation, the combination regimen of tal+tec is administered on a 28-day cycle, starting on Cycle 1 Day 1. For Dose Level 1 of the tal+tec combination regimen, the weekly treatment doses were 200 μg/kg talquetamab and 750 μg/kg teclistamab. Subsequent dose escalations occurred step-wise as follows: Dose Level 2 (200 μg/kg talquetamab and 1500 μg/kg teclistamab weekly): Dose Level 3 (400 μg/kg talquetamab and 1500 μg/kg teclistamab weekly); Dose Level 4 (800 μg/kg talquetamab and 1500 μg/kg teclistamab biweekly), and Dose Level 5 (800 μg/kg talquetamab and 3000 μg/kg teclistamab biweekly). An illustration of dose escalations is provided in FIG. 1.

The RP2R (recommended Phase 2 regimen) identified for Teclistamab was 3000 μg/kg every two weeks (Q2W) given subcutaneously, and the RP2R for Talquetamab was 800 μg/kg every two weeks (Q2W) given subcutaneously.

Talquetamab and teclistamab are administered as weight-based doses. Teclistamab is administered approximately 30 minutes before talquetamab.

Participants receive 3 step-up doses of talquetamab and teclistamab prior to Cycle 1 Day 1, as described in Table 14. Step-up doses are administered 2 to 4 days apart (intervals of <2 days or >4 days may be acceptable with sponsor approval).

TABLE 14
Step-up Doses for Talquetamab and Teclistamab

| Step-up Dose | Talquetamab Dose | | Teclistamab Dose | |
|---|---|---|---|---|
| 1 | 10 | μg/kg | 60 | μg/kg |
| 2 | 60 | μg/kg | 300 | μg/kg |
| 3 | 400 | μg/kg | 1500 | μg/kg |

Note: While the talquetamab Step-up Dose 3 was 300 μg/kg in Part 1 and 2, the dose of 400 μg/kg was identified as the RP2D based on PK, pharmacodynamics, safety, and efficacy findings, and is therefore used for the Step-up Dose 3 in Part 3.

The first treatment doses of talquetamab and teclistamab combination therapy are administered 2 to 4 days after the last step-up dose(s) and this represents Cycle 1 Day 1.
Talquetamab 800 μg/kg SC will be administered Q2W on Days 1 and 15 (±3 days) of a 28-day cycle.
Teclistamab 3000 μg/kg SC will be administered Q2W on Days 1 and 15 (±3 days) of a 28-day cycle

### Dosing Regimen for Part 3:

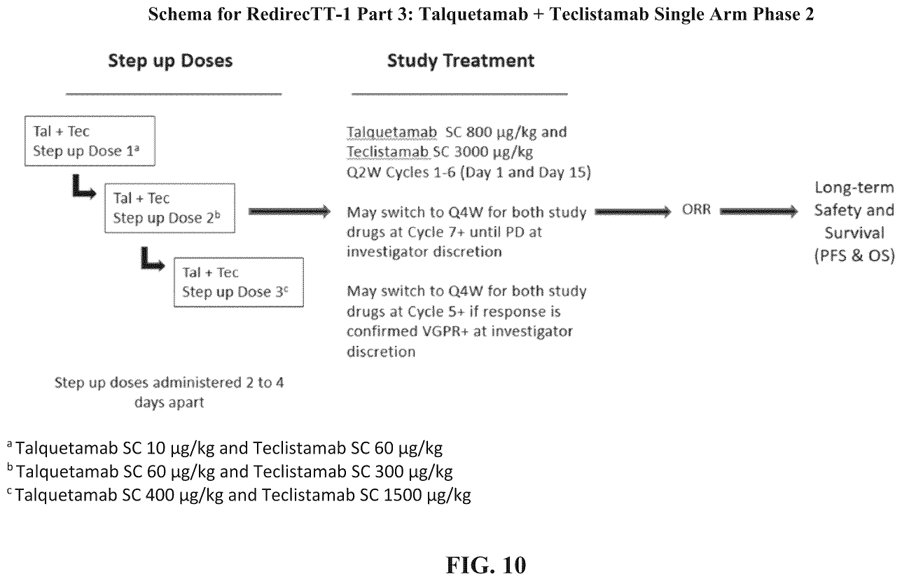

The tec+tal combination therapy will begin with 3 step-up doses prior to Cycle 1, Day 1. Teclistamab should be administered approximately 30 minutes before talquetamab. The first treatment doses will be administered 2 to 4 days after the last step-up dose(s) and this will represent Cycle 1 Day 1. Teclistamab 3000 μg/kg SC will be administered biweekly on Days 1 and 15 (±3 days) of a 28-day cycle, and talquetamab 800 μg/kg SC will be administered biweekly on Days 1 and 15 (±3 days) of a 28-day cycle. A change to Q4W may occur for both of the bispecific antibodies when a participant has achieved a confirmed response of VGPR or better and received a minimum of 4 cycles of therapy (ie, starting on Cycle 5), per investigator discretion. After completing 6 cycles of treatment (ie, starting on Cycle 7), the frequency of dosing may be decreased to Q4W for both bispecific antibodies, per investigator discretion. See FIG. 10.

### Arms & Interventions:

Experimental: Part 1: Dose Escalation. Participants receive tec+tal with or without daratumumab in 28-day cycles following initial step-up doses.

Experimental: Part 2: Dose Expansion. Participants receive treatment doses (combination of tal+tec and dara+tal+tec regimens) which will be determined by the RP2R(s) of the study treatment identified in Part 1.

Experimental: Part 3:

In Part 3, participants with RRMM and at least 1 focus of EMD will receive tal+tec combination therapy, which will begin with 3 step-up doses for both talquetamab and teclistamab (FIG. 10). To be eligible, participants with EMD must have documented RRMM with 1 or more focus of EMD not contiguous with a bone lesion at least ≥2 cm (at its greatest dimension) diameter on whole body PET-CT (or whole body MRI approved by sponsor), and not previously radiated.

### Primary Outcome Measures:

1. Part 1: Number of Participants with Dose Limiting Toxicity (DLT) [Time Frame: Up to 1 year and 6 months]. The dose limiting toxicities are based on drug related adverse events and defined as any of the following events: hematological or non-hematological toxicity of grade 3 or higher.
2. Part 1: Severity of DLT as Assessed by National Cancer Institute Common Terminology Criteria for Adverse Events (NCI-CTCAE) [Time Frame: Up to 1 year and 6 months]. Severity will be graded according to the National Cancer Institute Common Terminology Criteria for Adverse Events (NCI-CTCAE) version 5.0. Severity scale ranges from Grade 1 (Mild) to Grade 5 (Death). Grade 1=Mild, Grade 2=Moderate, Grade 3=Severe, Grade 4=Life-threatening, and Grade 5=Death related to adverse event.
3. Part 2: Number of Participants with Adverse Events (AEs) and Serious Adverse Events (SAEs) as a Measure of Safety and Tolerability [Time Frame: Up to 1 year and 6 months]. An AE is any untoward medical occurrence in a clinical study participant administered a medicinal (investigational or non-investigational) product. An AE does not necessarily have a causal relationship with the intervention. SAE is any AE that results in: death, persistent or significant disability/incapacity, requires inpatient hospitalization or prolongation of existing hospitalization, is life-threatening experience, is a congenital anomaly/birth defect, and suspects transmission of any infectious agent via a medicinal product.
4. Part 2: Number of Participants with Adverse Events and SAEs by Severity [Time Frame: Up to 1 year and 6 months]. Severity will be graded according to the National Cancer Institute Common Terminology Criteria for Adverse Events (NCI-CTCAE) version 5.0. Severity scale ranges from Grade 1 (Mild) to Grade 5 (Death). Grade 1=Mild, Grade 2=Moderate, Grade 3=Severe, Grade 4=Life-threatening, and Grade 5=Death related to adverse event.

### Objectives and Endpoints:

Part 1 and Part 2: The primary objective for Part 1 is to identify the RP2R(s) for each treatment combination by assessing the incidence and severity of DLTs. The primary objective for Part 2 is to characterize the safety of each RP2R for the selected treatment combination(s) by assessing the incidence and severity of AEs and SAEs. The RP2R(s) will describe the combination doses and schedules of the treatment combinations to be pursued in Phase 2. A key secondary objective for both parts is to characterize the pharmacokinetics of the study treatments. For Part 2, an additional key secondary objective is to evaluate the preliminary anticancer activity at RP2R(s).
Part 3: The primary objective for Part 3 is to evaluate the anticancer activity of tal+tec combination therapy at the RP2R in participants with relapsed or refractory multiple myeloma and EMD by assessing the ORR. Key secondary objectives are to evaluate attributes of response, PFS, and OS, and to further characterize the safety of the combination therapy.

### Eligibility Criteria:

### Inclusion Criteria Include the Following:

Documented initial diagnosis of multiple myeloma according to International Myeloma Working Group (IMWG) diagnostic criteria based on documented medical history

### Parts 1 and 2:

Participant could not tolerate or has disease that is relapsed or refractory to established therapies, including the last line of therapy (except as noted in point ‘a’ and ‘b’). (a) For cohorts without daratumumab, prior lines of therapy must include a proteasome inhibitor (PI) (example, bortezomib, carfilzomib, ixazomib), an immunomodulatory drug (IMiD) (example, thalidomide, lenalidomide, pomalidomide), and an anti-CD38 therapy (example, daratumumab, isatuximab) in any order. (b) For cohorts with daratumumab, prior lines of therapy must include a PI (example, bortezomib, carfilzomib, ixazomib) and an IMiD (example, thalidomide, lenalidomide, pomalidomide). Treatment with an anti-CD38 therapy (example, daratumumab) is allowed greater than equal to (>=) 90 days prior to study treatment if the participant did not discontinue prior treatment due to adverse events related to anti-CD38 therapy.

### Part 3:

a. Relapsed or refractory disease, and exposed to a PI, IMiD, and an anti-CD38 mAb. Exposed as defined by: Participant must have undergone ≥1 complete cycle of treatment for each regimen, unless progressive disease was the best response to the regimen or if the regimen was discontinued due to significant toxicity.
b. Documented evidence of progressive disease based on investigator's determination of response by IMWG criteria on or after their last regimen. Relapsed or refractory disease as defined below:
Relapsed disease is defined as an initial response to prior treatment, followed by confirmed progressive disease by IMWG criteria >60 days after cessation of treatment.
Refractory disease is defined as <25% reduction in M-protein or confirmed progressive disease by IMWG criteria during previous treatment or ≤60 days after cessation of treatment.

### Parts 1 and 2:

Measurable disease at screening as defined by at least 1 of the following:
a. Serum M-protein level ≥1.0 g/dL, or
b. Urine M-protein level ≥200 mg/24 hours, or
c. Light chain multiple myeloma, for participants without measurable disease in the serum or urine: serum immunoglobulin FLC ≥10 mg/dL and abnormal serum Ig kappa-lambda FLC ratio, or
d. For participants without measurable disease in the serum, urine, or involved FLC, presence of plasmacytomas (2 cm).
   For Part 1 and Part 2 Cohorts with EMD Participants:
Documented relapsed or refractory multiple myeloma with 1 or more focus of EMD meeting the following criterion: any extramedullary plasmacytoma not contiguous with a bone lesion ≥2 cm (at its greatest dimension) diameter on PET-CT and not previously radiated.

### Part 3:

Documented relapsed or refractory multiple myeloma with 1 or more focus of EMD meeting the following criteria: extramedullary plasmacytoma not contiguous with a bone lesion at least 1 lesion ≥2 cm (at its greatest dimension) diameter on whole body PET-CT (or whole body MRI approved by sponsor), and not previously radiated.
NOTE: In exceptional circumstances, whole body PET-CT (or whole body MRI) with local institutional imaging read confirming at least 1 EMD focus ≥3 cm may be considered eligible with sponsor approval.
Part 1 and Part 2: ECOG performance status grade of 0 or 1 at screening and immediately before the start of study drug administration.
Part 3: ECOG performance status grade of 0, 1, or 2 at screening and immediately before the start of study drug administration.
All Parts: A participant of childbearing potential must have a negative serum pregnancy test at screening and within 24 hours of the start of the study treatment and must agree to further serum or urine pregnancy tests during the study.

### Exclusion Criteria Include the Following:

All Parts:
Prior or concurrent exposure to any of the following in the specified time frame prior to enrollment:
   a. Targeted therapy, epigenetic therapy, or treatment with an investigational treatment or an invasive investigational medical device within 21 days or at least 5 half-lives, whichever is less
   b. Investigational vaccine other than SARS-CoV-2 vaccine approved/in use under emergency approval within 4 weeks
   c. Monoclonal antibody therapy within 21 days
   d. Cytotoxic therapy within 21 days
   e. PI therapy within 14 days
   f. IMiD agent therapy within 7 days
   g. Radiotherapy within 21 days. However, if the radiation portal covered ≤5% of the bone marrow reserve, the participant is eligible irrespective of the end date of radiotherapy
   h. Gene modified adoptive cell therapy (eg, chimeric antigen receptor modified T cells, NK cells) within 3 months.

### Part 3:

i. Cannot have received a prior BCMA targeted bispecific antibody therapy
j. Cannot have received any prior GPRC5D targeted therapy
All Parts: A cumulative dose of corticosteroids equivalent to ≥140 mg of prednisone within 14 days before the first dose of study drug (does not include pretreatment medications)
All Parts: Live, attenuated vaccine within 4 weeks prior to first dose of study drug unless approved by sponsor. Non-live or non-replicating vaccines authorized for emergency use (eg, COVID-19) are allowed
Active hepatitis C infection as measured by positive hepatitis C virus (HCV)-RNA testing. Participants with a history of HCV antibody positivity must undergo HCV RNA testing
Known allergies, hypersensitivity, or intolerance to daratumumab, talquetamab, teclistamab, or their excipients

Enrolled pts had MM per International Myeloma Working Group 2016 criteria; were RR or intolerant to the last line of therapy (LOT); were exposed to a proteasome inhibitor, immunomodulatory drug, and anti-CD38 therapy; and had measurable disease. All pts provided informed consent. The primary objectives are to evaluate safety and to identify a recommended phase 2 regimen (RP2R) for the combination. Responses were investigator assessed. AEs were graded per CTCAE v5.0. Cytokine release syndrome (CRS) and immune effector cell-associated neurotoxicity syndrome (ICANS) were graded per ASTCT criteria.

### Results as of Data Cut-Off Dec. 12, 2022:

Sixty-three (63) pts received tec+tal. Median (range) age was 67 y (39-81); median (range) prior lines of therapy (LOT) was 5 (1-11); 33% (15/45) had high-risk cytogenetics; 78% (49/63) were triple-class refractory: 63% (40/63) were penta-drug exposed; and 43% (27/63) had extramedullary disease (EMD; all bone independent). Median (range) duration of follow-up was 14.4 mos (0.5-21.9). The most common treatment-emergent AEs were CRS (81%; grade [gr] 3, 3%, no gr 4), neutropenia (76%; gr 3/4, 75%), and anemia (60%; gr 3/4, 43%). Dose-limiting toxicities (DLTs) were reported at dose level 1 (gr 3 herpetic stomatitis) and dose level 3 (gr 3 AST/ALT elevation). One ICANS event was reported at dose level 3. No DLTs were reported at the RP2R. Across all dose levels, overall response rate (ORR) was 84% (52/62) among all evaluable pts and 73% (19/26) among evaluable pts with EMD; rate of CR or better (≥CR) was 34% (21/62) and 31% (8/26), respectively. At the RP2R. ORR was 92% (12/13) among all evaluable pts and 83% (5/6) among evaluable pts with EMD; rate of ≥CR was 31% (4/13) and 33% (2/6), respectively.

Summary/Conclusion: In this first combination study of a BCMA- and GPRC5D-targeted bispecific antibody, tec+tal at the RP2R has a manageable safety profile consistent with each of the monotherapies. A 92% ORR was observed in pts with advanced RRMM at the RP2R and an ORR of 83% was achieved in pts with EMD, a high-risk population with unmet need.

### Results as of Data Cut-Off Mar. 16, 2023:

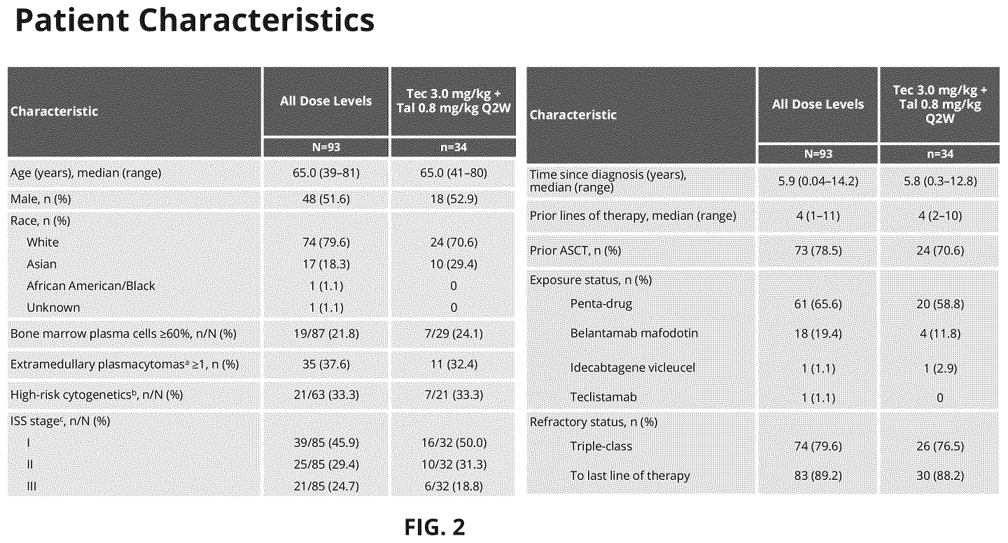

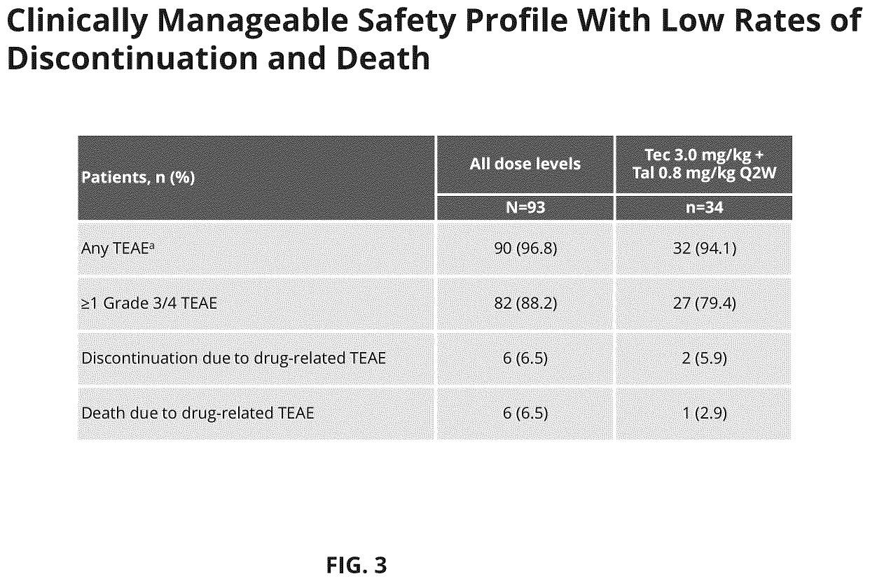

Characteristics of patients enrolled in the RedirecTT-1 trial are summarized in FIG. 2, which shows, for example, that enrolled patients had a median of 4 prior lines of therapy (1-11 prior lines for patients treated at all dose levels and 2-10 prior lines for patients treated at the RP2R). Many patients enrolled in the trial had received prior autologous stem cell transplant (ASCT) (78.5% of patients treated at all dose levels and 70.6% of patients treated at the RP2R). Safety results are summarized in FIGS. 3-7.

As shown in FIG. 3, the combination regimen had a clinically manageable safety profile with low rates of discontinuation and death. TEAEs (treatment-emergent adverse events) are defined as new or worsening events occurring in the safety population at or after the first drug treatment up to and including 30 days after last dose of study drug or the day prior to start of subsequent therapy (whichever comes first). Adverse events (AEs) starting more than 30 days after last dose of study drug that are determined to be related to the study drug are also considered TEAEs. All deaths due to drug-related TEAEs (treatment-emergent adverse events) were due to infections.

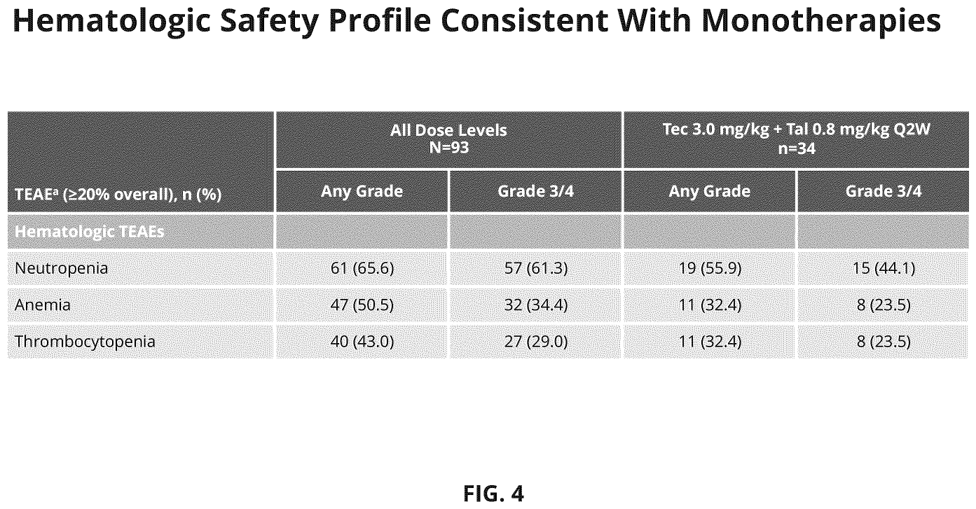

As shown in FIG. 4, the hematologic safety profile was consistent with Tec and Tal monotherapies. There were no discontinuations due to hematologic TEAEs. Febrile neutropenia occurred in 12.9% of patients across all dose levels, including 8.8% at the RP2R.

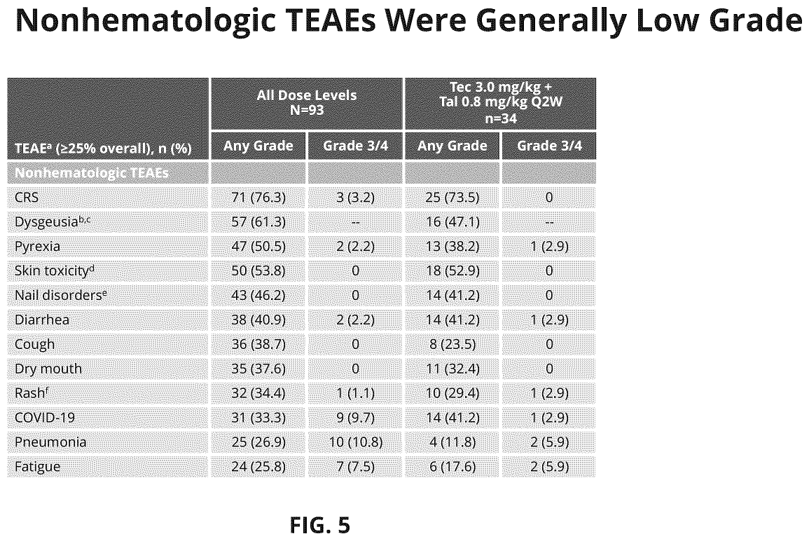

As shown in FIG. 5, non-hematologic TEAEs were generally low grade. Rates of grade 3/4 nonhematologic TEAEs were low overall, including at the RP2R 5 ICANS events in 3 patients; maximum severity grade 3. At the RP2R: 2 events in 1 patient; maximum severity grade 1. The AE profile was consistent with each of the drugs as monotherapy.

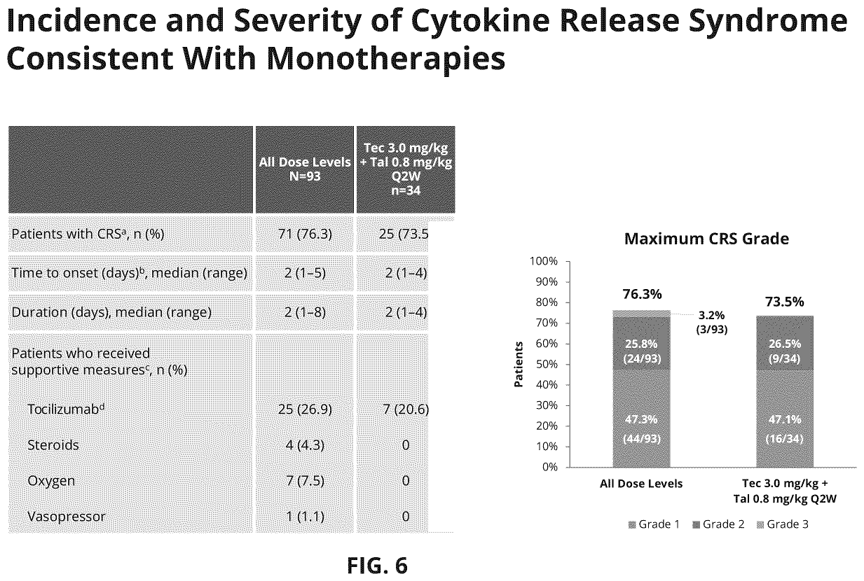

As shown in FIG. 6, incidence and severity of cytokine release syndrome (CRS) was consistent with Tec and Tal monotherapies. The majority of CRS events occurred during step-up dosing or cycle 1. All CRS events resolved or were resolving at data cutoff.

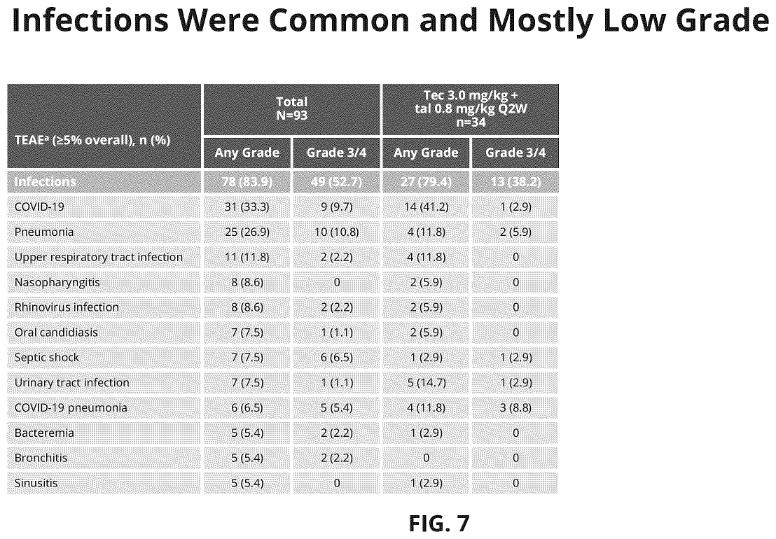

As shown in FIG. 7, infections were common and mostly low grade. Most infections were grade 1 or 2: 81.7% with ≥1 post-baseline IgG value <400 mg/dL or hypogammaglobulinemia TEAE all grade 1 or 2 (85.3% at the RP2R).

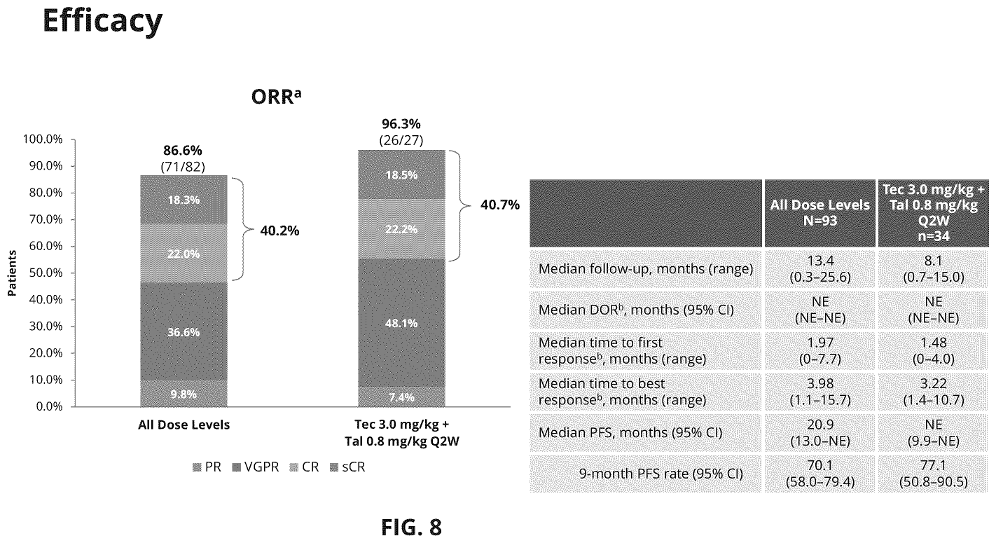

As shown in FIG. 8, the overall response rate (ORR) ORR was high 86.6%) across dose levels studied and 96.3% at the RP2R. At data cutoff, 61% (57/93) of patients remained on treatment.

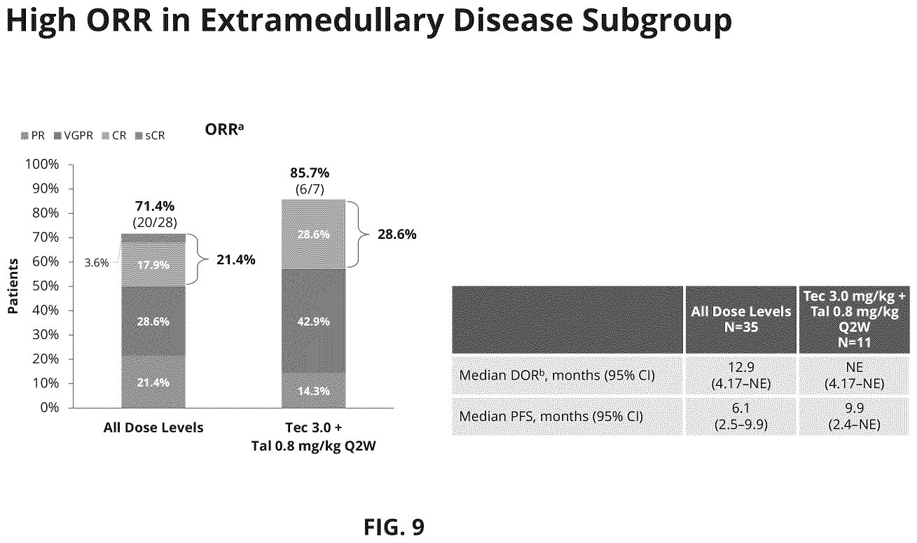

As shown in FIG. 9, a high ORR was observed in the EMD subgroup. All were soft tissue plasmacytomas. At the RP2R (n=11) median follow-up of 7.2 months (range 0.7-14.2), 85.7% (6/7) ORR was observed; and 28.6% (2/7) Complete Response or Stringent Complete Response was observed.

In summary, at a data cut-off of Mar. 16, 2023, a 96% ORR was observed with Q2W Tec+Tal at RP2R in triple-class exposed MM, competitive with current CAR T-cell therapies. A notable 85.7% ORR was observed in patients with extramedullary soft-tissue plasmacytomas, with median DOR not reached at 7.2 months. The safety profile of the combination was consistent with monotherapies. No new or additive toxicity were seen for either teclistamab or talquetamab.

### Updated RedirecTT-1 Study Methods, Results and Discussion, as of March 2024:

### Treatment

Patients received escalating subcutaneous talquetamab plus teclistamab in 28-day cycles. Step-up doses, adapted from schedules used with each monotherapy to mitigate severe CRS, were administered 2-4 days apart and prior to full treatment doses. Step-up and full treatment doses were administered on the same day, 30 (±10) minutes apart. Hospitalization and pretreatment with dexamethasone, diphenhydramine, and acetaminophen were required prior to all step-up and first treatment doses. Patients could switch from QW to Q2W and from Q2W to monthly (Q4W) talquetamab and teclistamab dosing after achieving a partial response or better from cycle 5. Prophylactic measures for infections were recommended and administered per institutional guidelines. Immunoglobulin replacement was protocol recommended to maintain serum immunoglobulin G levels above 400 mg/dL regardless of active or history of infection. Intravenous immunoglobulin was recommended at a dose of 0.4 g/kg every 3 to 6 weeks; immunoglobulin G levels were recommended to be measured at least every 3 months after reaching steady state. For serious or recurrent/chronic infections, immunoglobulin replacement was administered per institutional guidelines. Patients received study treatment until unacceptable toxicity, consent withdrawal, confirmed disease progression, death, investigator or sponsor decision to discontinue treatment, or study end.

### End Points and Assessments

The primary objective of phase 1 dose escalation was to identify a talquetamab plus teclistamab dose for phase 2 expansion by assessing adverse events and dose-limiting toxicities. Secondary end points included overall response, duration of response, time to response, pharmacokinetics, pharmacodynamics, and immunogenicity. Progression-free survival was also assessed. Response was investigator-assessed per IMWG 2016 criteria. Best response was reported (both confirmed and unconfirmed). EMD was assessed by physical examination every 4 weeks and radiologic assessment every 12 weeks. Adverse events were graded per Common Terminology Criteria for Adverse Events v5.0 and were recorded up to 30 days after the patient last received study treatment. CRS and immune effector cell-associated neurotoxicity syndrome (ICANS) were graded per American Society for Transplantation and Cellular Therapy guidelines. Blood and serum samples were obtained for pharmacokinetic, pharmacodynamic, and immunogenicity analyses.

### Statistical Analysis

Safety, pharmacokinetics, pharmacodynamics, and immunogenicity were assessed in patients who received ≥1 treatment dose. All treated patients with ≥1 postbaseline disease assessment were included in the estimation of overall response, in addition to those who died, progressed, or discontinued study treatment (irrespective of whether patients had ≥1 postbaseline disease assessment). Response was calculated with corresponding exact 95% confidence intervals. All treated patients were evaluated for time to response and progression-free survival. The Kaplan-Meier method was used to estimate time to response, duration of response, and progression-free survival. All data were analyzed using SAS v9.4. No formal statistical hypothesis testing was performed.

### Results

Between Dec. 9, 2020, and Apr. 26, 2023, 116 patients underwent screening. As of Mar. 15, 2024, 94 patients received talquetamab plus teclistamab, including 44 who received talquetamab 0.8 mg/kg plus teclistamab 3.0 mg/kg Q2W (dose level 5), which was selected for phase 2 expansion. Median follow-up was 20.3 months (range, 0.5-37.1) overall and 18.2 months (range, 0.7-27.0) at dose level 5. Forty-nine (50.1%) patients remain on talquetamab plus teclistamab, while 1 patient remains on teclistamab monotherapy. Across all doses, patients received a median of 10 (range, 1-39) talquetamab cycles and 12 (range, 1-39) teclistamab cycles.

Patients had a median age of 64.5 years (range, 39-81) and had received a median of 4 (range, 1-11) prior lines of therapy over a median duration of 6.1 years (range, 0.3-14.6) since diagnosis. All patients were triple-class exposed and most were penta-drug exposed (n=61 [64.9%]). Seven patients received prior bispecific antibodies and 4 received prior CAR-T therapy. Eighty-seven (92.6%) patients were refractory to their last line of therapy and 81 (86.2%) were triple-class refractory. Thirty-four (36.2%) patients had EMD.

### Safety

Three patients had dose-limiting toxicities, 1 patient each treated with talquetamab 0.2 mg/kg plus teclistamab 0.75 mg/kg QW (grade 3 oral herpes), talquetamab 0.4 mg/kg plus teclistamab 1.5 mg/kg QW (grade 3 elevated alanine aminotransferase/aspartate aminotransferase), and dose level 5 (grade 4 thrombocytopenia). Across all dose levels, all patients had ≥1 adverse event; 90 (95.7%) patients had 21 grade 3/4 event. The most common adverse events were CRS, neutropenia, taste changes, and nonrash skin adverse events; hematologic adverse events (neutropenia, anemia, and thrombocytopenia) were the most common grade 3/4 events. Eighty-seven (92.6%) patients had cycle delays or dose modifications due to adverse events (64 [68.1%] due to infections). Adverse events led to discontinuation of one or both agents in 15 (16.0%) patients (12 [12.8%] patients discontinued due to the event leading to death). Seven (7.4%) patients discontinued one or both argents due to drug-related adverse events (5 [5.3%] due to infections). Fourteen (14.9%) patients died due to adverse events (11 [11.7%] died due to infections); there were 6 drug-related grade 5 adverse events. Four (4.3%) patients died due to progressive disease.

Taste changes, encompassing taste disorder, dysgeusia, hypogeusia, and ageusia, occurred in 61 (64.9%) patients, and led to cycle delays or dose modifications in 5 (5.3%) and discontinuation in 1 (1.1%). Rash and nonrash skin adverse events occurred in 37 (39.4%) and 57 (60.6%) patients, respectively; all were grade 1/2, except for one grade 3 rash adverse event at dose level 5. Rashes led to cycle delays or dose modifications in 2 (2.1%) patients; no patients discontinued due to rash or nonrash skin adverse events. Nail-related adverse events occurred in 49 (52.1%) patients, all were grade 1/2, and none led to cycle delays, dose modifications, or discontinuations.

Infections occurred in 84 (89.4%) patients and were grade 3/4 in 60 (63.8%). Forty-seven (50.0%) patients received antiviral prophylaxis for herpes and 11 (11.7%) for Pneumocystis jirovecii pneumoniae. Fifty-nine (62.8%) patients had a COVID-19 vaccination. Thirty-eight (40.4%) patients had COVID-19 infections, with 17 (18.1%) having a grade 3/4 event; 2 (2.10%) patients had a fatal COVID-19 adverse event. Onset of most grade ≥3 infections was within the first 12 months of treatment (65 [69.1%] patients). Opportunistic infections occurred in 10 (10.6%) patients. At baseline, 37 (39.4%) patients had hypogammaglobulinemia defined as immunoglobulin G values <400 mg/dL, whereas 30 (31.9%) patients had post-treatment hypogammaglobulinemia; assessments were censored for patients with immunoglobulin G myeloma and after intravenous immunoglobulin replacement. Intravenous immunoglobulin was given to 38 (40.4%) patients.

CRS occurred in 74 (78.7%) patients and was mostly low-grade; grade 3 events occurred in 2 (2.1%) patients. CRS led to cycle delays or dose modifications in 14 (14.9%) patients. Median time to onset and duration of CRS were each 2.0 days. Sixty-one (64.9%) patients received supportive measures for CRS, including tocilizumab in 24 (25.5%; 9, 13, and 2, respectively, for grade 1, 2, and 3 events). ICANS occurred in 3 (3.2%) patients, with one grade 3 event reported; events occurred during step-up doses, had a median duration of 3.0 days, and all events recovered.

### Efficacy

Across all doses, responses occurred in 77/94 (81.9%) patients, including 23/34 (67.6%) with EMD. Median time to first response was 1.8 months (range, 0.3-7.7) overall and 2.8 months (range, 1.4-5.1) in patients with EMD. Seventy-two (76.6%) patients had a very good partial response or better and 46 (48.9%) had a complete response or better; in patients with EMD, 20 (58.8%) had a very good partial response or better and 10 (29.4%) had a complete response or better. Responses were consistent across clinically relevant subgroups.

At dose level 5, responses occurred in 36/44 (81.8%) patients and in 12/18 (66.7%) with EMD. Median time to first response was 1.5 months (range, 0.3-5.1) overall and 3.0 months (range, 1.4-5.1) in patients with EMD. Deep responses were observed in patients with and without EMD treated at dose level 5. Responses were consistent across clinically relevant subgroups at this dose level.

The probability of remaining in response at 12 and 18 months was 85.6% (95% CI, 74.9-92.0) and 76.6% (95% CI, 64.0-85.3) across all doses and 91.0% (95% CI, 74.6-97.0) and 85.9% (95% CI, 65.6-94.7) at dose level 5. In patients with EMD, the probability of remaining in response at 12 and 18 months was 70.0% (95% CI, 45.1-85.3) and 52.5% (95% CI, 25.1-74.1) across all doses and 81.8% (95% CI, 44.7-95.1) at both timepoints for dose level 5. In patients who switched from dose level 5 to monthly dosing (n=28), 27 (96.4%) maintained or deepened response.

Across all dose levels, 12- and 18-month progression-free survival was 70.7% (95% CI, 60.0-79.0) and 62.2% (95% CI, 50.8-71.7) in all patients and 45.5% (95% CI, 27.7-61.7) and 34.1% (95% CI, 16.5-52.7) in patients with EMD. At dose level 5, 12- and 18-month progression-free survival was 73.7% (95% CI, 57.4-84.5) and 69.8% (95% CI, 52.4-81.8) in all patients and 52.9% (95% CI, 27.6-73.0) at both timepoints in patients with EMD.

Pharmacokinetics. Immunogenicity, and Pharmacodynamics

At dose level 5, talquetamab and teclistamab exposures were comparable with observations from each agent as monotherapy. Across all dose levels, no anti-talquetamab or anti-teclistamab antibodies were detected post-dosing.

T-cell activation was similar across all doses, although variability was high. At dose level 5. T-cell activation peaked prior to the first full treatment dose and was maintained thereafter.

Across all dose levels, median soluble BCMA concentration at baseline was 87.3 ng/ml (range, 4.708-4017) in responders and 210.1 ng/ml (range, 5.015-1224) in nonresponders. Decreases in soluble BCMA concentration from baseline to cycle 3 day 1 were observed in 89% (51/57) of responders (median change: −91%; range, −99-471%) and 50% (2/4) of nonresponders (median change: 62%; range, −98-1719%) across all dose levels.

### Discussion

Talquetamab plus teclistamab had a similar safety profile with each agent as monotherapy. The incidence of grade 3/4 infections was 63.8% across all dose levels; corresponding rates were 55.2% with teclistamab monotherapy and 19.6% and 14.5%, respectively, with the 0.4 mg/kg QW and 0.8 mg/kg Q2W talquetamab monotherapy schedules. Compared with initial results from this study (Mar. 16, 2023, data cutoff; median follow-up of 13.4 months), no additional deaths were observed due to drug-related adverse events with longer median follow-up (20.3 months). The rates of treatment-related adverse events leading to death or discontinuation were low and were mostly due to infections. Prophylactic infection measures were recommended and administered as per institutional guidelines. The protocol recommended immunoglobulin replacement to maintain serum immunoglobulin G levels above 400 mg/dL, irrespective of prior or active infection, with close monitoring of hypogammaglobulinemia. Overall, these results reinforce the importance of infection screening, prophylaxis, management, and close monitoring for hypogammaglobulinemia and neutropenia.

CRS was common and mostly low-grade (no grade 4/5 events were observed), consistent with T-cell-redirection therapies, including talquetamab and teclistamab monotherapies. ICANS was rare, typically low-grade, and reversible with standard mitigation measures. GPRC5D-associated adverse events were common, low-grade, led to few dose modifications and discontinuations, and were managed per protocol recommendations.

Evaluation of overall response at a prior data cutoff included all treated patients with 21 postbaseline disease assessment (excluding those who died/discontinued too early for evaluability), leading to a less conservative response rate. At the data cutoff reported here across all doses and at dose level 5, responses were observed in 81.9% and 81.8% of patients, respectively, which is highly promising. Responses with approved T-cell direction therapies were observed in 74.1% and 71.7% of patients, respectively, with talquetamab 0.4 mg/kg QW and 0.8 mg/kg Q2W, 63.0% with teclistamab, 61.0% with elranatamab, 83.0% with ciltacabtagene autoleucel, and 67.0% with idecabtagene vicleucel. The proportion of patients who achieved a complete response or better was high with talquetamab plus teclistamab across all doses (48.9%) and was particularly deep at dose level 5 (52.3%), which is promising in the context of teclistamab (45.5%) and talquetamab monotherapy (33.6% with 0.4 mg/kg QW and 38.7% with 0.8 mg/kg Q2W). At dose level 5, responses were highly durable, with a probability of patients remaining in response at 18 months of 85.9%. Eighteen-month duration of response was 36.7% and 48.7%, respectively, with talquetamab 0.4 mg/kg QW and 0.8 mg/kg Q2W monotherapy, and 57.1% with teclistamab monotherapy. Responses were consistent across clinically relevant subgroups, building confidence in the utility of this off-the-shelf combination across patient populations, including high-need subgroups.

BCMA-targeting CAR-T therapies have demonstrated high rates of response in patients with EMD, although patient populations have included those with paramedullary lesions, who have a better prognosis than patients with EMD. Durability of response and progression-free survival with CAR-T therapies were shorter in patients with EMD compared with the overall study populations. Similarly, for patients with EMD treated with standard-of-care regimens, the proportion of patients achieving a response was lower and progression-free survival was shorter than in patients without EMD. In patients with EMD, responses with talquetamab monotherapy were 45.5% (0.4 mg/kg QW) and 40.0% (0.8 mg/kg Q2W) and 37.5% with teclistamab monotherapy. In RedirecTT-1, 66.7% of patients with EMD had a response at dose level 5 (33.3% had a complete response or better) and the probability of remaining in response at 18 months was 81.8%.

Study limitations include the short follow-up and small overall sample size; however, this study enrolled a higher proportion of patients with EMD compared with other studies of antimyeloma therapies. RedirecTT-1 is an open-label, nonrandomized phase 1b/2 study and no comparative analyses were possible.

In this first study combining two T-cell-redirecting bispecific antibodies, talquetamab plus teclistamab had a similar safety profile to each agent as monotherapy. Responses were observed across investigated dose levels and were particularly deep and durable at dose level 5, including in patients with EMD. Based on these results, this novel, dual-targeting, off-the-shelf combination warrants further investigation at dose level 5 in patients with EMD, and at other dose levels for patients without EMD.

### Example 2: A Study Comparing Talquetamab Plus Pomalidomide, Talquetamab Plus Teclistamab, and Elotuzumab, Pomalidomide, and Dexamethasone or Pomalidomide, Bortezomib, and Dexamethasone in Participants with Relapsed or Refractory Myeloma Who have Received an Anti-CD38 Antibody and Lenalidomide (MonumenTAL-6)

This is a randomized, Phase 3, active-controlled, parallel, multicenter, interventional, open-label study in participants with relapsed or refractory multiple myeloma who have received 1 to 4 prior lines of therapy including an anti-CD38 mAb and lenalidomide.

### Antibodies

Talquetamab and teclistamab are made by Janssen Pharmaceuticals. Sequences of the antibodies are further described in Example 1 above.

### Objectives

This is a Phase 3 Randomized Study Comparing Talquetamab in Combination with Pomalidomide (Tal-P), Talquetamab in Combination with Teclistamab (Tal-Tec), and Investigator's Choice of Either Elotuzumab, Pomalidomide, and Dexamethasone (EPd) or Pomalidomide, Bortezomib, and Dexamethasone (PVd) in Participants with Relapsed or Refractory Myeloma who Have Received 1 to 4 Prior Lines of Therapy Including an Anti-CD38 Antibody and Lenalidomide.

The primary objective of this study is to compare the effectiveness of either talquetamab plus pomalidomide (Tal-P) or talquetamab plus teclistamab (Tal-Tec) with elotuzumab, pomalidomide, and dexamethasone (EPd) or pomalidomide, bortezomib, and dexamethasone (PVd). Secondary objectives include to further compare the efficacy of either Tal-P or Tal-Tec with EPd or PVd and to assess the safety and tolerability of Tal-P and Tal-Tec. The primary hypothesis is that Tal-P or Tal-Tec will improve PFS compared with EPd or PVd in participants with relapsed or refractory multiple myeloma who have received 1 to 4 prior lines of therapy including an anti-CD38 mAb and lenalidomide.

A target of approximately 795 participants randomized in a 1:1:1 ratio to receive Tal-P (ArmA), Tal-Tec (ArmB), or EPd or PVd (ArmC), with stratification by number of prior lines of therapy (1 versus ≥2), ISS stage at screening (I versus II/III), and investigator's intended choice of therapy (EPd versus PVd).

### Study Arms and Duration

Study treatment will be administered in 28-day cycles for Tal-P (Arm A), Tal-Tec (Arm B), and EPd (Arm C). For PVd (Arm C), study treatment will be administered in 21-day cycles. At the time of screening, based on the investigator's choice, they must declare whether the participant will be treated with EPd or PVd should they be randomized to Arm C.

### Arm B (Tal-Tec) Dosing Schedule

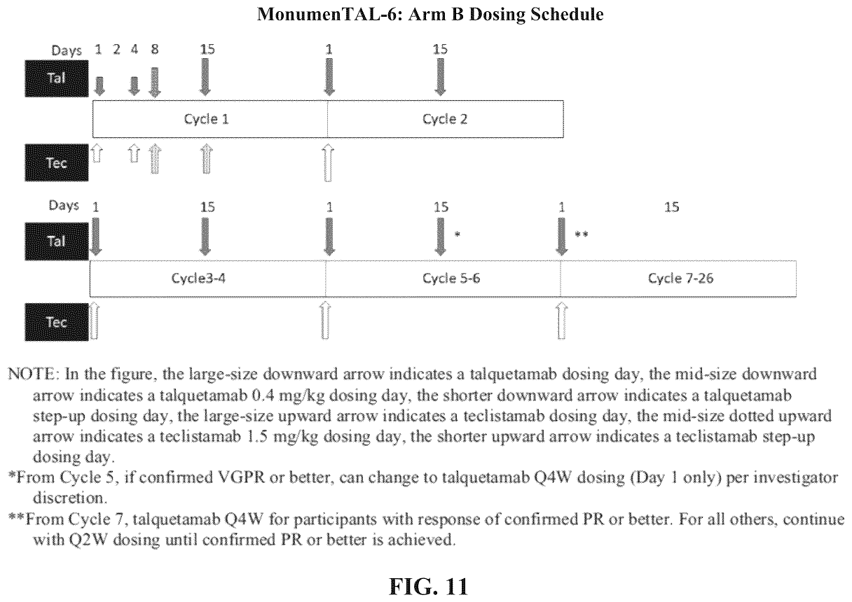

In Arm B, participants will receive therapy with Tal/Tec (as appropriate) for up to 26 cycles if they have no sign of progressive disease or toxicity. An initial Arm B dosing schedule was later updated in a protocol amendment, as a result of developing data from other clinical studies involving Tal and Tec. For example, the RedirecTT-1 study's exploratory E-R analysis demonstrated a trend for improved depth of response associated with Q2W talquetamab dosing in the initial treatment cycles. A reduction in talquetamab dose frequency after response does not compromise efficacy and may ameliorate GPRC5D specific AEs. Monthly administration of teclistamab at 3 mg/kg at Cycle 2 is estimated to achieve comparable exposure to 1.5 mg/kg Q2W based on Study 64007957MMY1001 (MajesTEC-1) and study RedirecTT-1 data. Moreover, there may be improved tolerability with lower frequency dosing while maintaining response. Both the initial Arm B dosing schedule and updated Arm B dosing schedule are provided below. The updated Arm B dosing schedule is illustrated in FIG. 11.

### Talquetamab: Initial Arm B Dosing Schedule:

In Cycle 1 (28-day cycle) of Arm B, the following doses of subcutaneous talquetamab will be administered:
Talquetamab step-up dose 1 (SU1): 0.01 mg/kg, administered on Day 1;
Talquetamab step-up dose 2 (SU2): 0.06 mg/kg, administered ≥2 d after SU1, between Days 3-6;
Talquetamab treatment dose (sometimes referred to as step-up dose 3): 0.4 mg/kg, administered ≥2 d after SU2, between Days 5-10;
Talquetamab treatment dose: 0.8 mg/kg, administered ≥2 d after first treatment dose (step-up dose 3) of 0.4 mg/kg, between Days 7-15.

In subsequent treatment cycles following Cycle 1 (28-day treatment cycles), the subcutaneous Talquetamab treatment dose of 0.8 mg/kg will be administered as follows in Arm A:
Cycle 2-Cycle 4 (C2-C4), Talquetamab SC is administered bi-weekly (Q2W), i.e., 14 days (±3 d) after prior treatment dose;
From Cycle 5 (C5), if confirmed VGPR or better, schedule can change to Q4W dosing (Day 1 of each 28-day cycle only) per investigator discretion;
Q4W from Cycle 7 (C7) for all participants.

### Teclistamab: Initial Arm B Dosing Schedule:

In Cycle 1 (28-day cycle) of Arm B, the following doses of subcutaneous Teclistamab (Tec) will be administered:
Tec step-up dose 1 (SU1): 0.06 mg/kg, administered on Day 1;
Tec step-up dose 2 (SU2): 0.3 mg/kg, administered ≥2 d after SU1, between Days 3-6;
Tec treatment dose (sometimes referred to as step-up dose 3): 1.5 mg/kg, administered ≥2 d after SU2, between Days 5-10;
Tec treatment dose: 3.0 mg/kg, administered ≥2 d after first treatment dose (step-up dose 3) of 1.5 mg/kg, between Days 7-15.

In subsequent treatment cycles following Cycle 1 (28-day treatment cycles), the subcutaneous Tec treatment dose of 3.0 mg/kg will be administered as follows:
Cycle 2 Tec is administered 14 days (±3 d) after prior treatment dose;
From Cycle 3 (C3), if confirmed partial response (PR) or better, schedule can change to Q4W dosing (Day 1 of each 28-day cycle only) per investigator discretion;
Q4W from Cycle 7 (C7) for all participants.

### Talquetamab: UPDATED Arm B Dosing Schedule:

Talquetamab will be administered as follows, in 28-day treatment cycles;
In Cycle 1, the following doses of subcutaneous talquetamab are administered:
   Talquetamab step-up dose 1 (SU1): 0.01 mg/kg, administered on Day 1;
   Talquetamab step-up dose 2 (SU2): 0.06 mg/kg, administered ≥2 d after SU1;
   Talquetamab treatment dose (sometimes referred to as step-up dose 3): 0.4 mg/kg, administered ≥2 d after SU2;
   Talquetamab treatment dose: 0.8 mg/kg, administered ≥2 d after first treatment dose (step-up dose 3) of 0.4 mg/kg, between Days 7-15,
In subsequent treatment cycles following Cycle 1, the subcutaneous Talquetamab treatment doses of 0.8 mg/kg are administered as follows:
   Cycle 2-Cycle 4 (C2-C4). Talquetamab SC is administered bi-weekly (Q2W), i.e., 14 days (±3 d) after prior treatment dose;
   From Cycle 5 (C5), if confirmed VGPR or better, schedule can change to Q4W dosing (Day 1 of each 28-day cycle only), with the change to Q4W occurring on Cycle 5 or Cycle 6 Day 1 (±3 d);
   At Cycle 7 Day 1 (±3 d), if confirmed PR or better, schedule must change to Q4W dosing;
   At Cycle 7 Day 1 (±3 d), if not in confirmed PR or better, continue with Q2W dosing until confirmed PR or better is achieved.

### Teclistamab: UPDATED Arm B Dosing Schedule:

Teclistamab will be administered as follows, in 28-day treatment cycles.
In Cycle 1, the following doses of subcutaneous teclistamab are administered:
   Tec step-up dose 1 (SU1): 0.06 mg/kg, administered on Day 1;
   Tec step-up dose 2 (SU2): 0.3 mg/kg, administered ≥2 d after SU1;
   Tec treatment dose (sometimes referred to as step-up dose 3): 1.5 mg/kg, administered ≥2 d after SU2;
   Tec treatment dose: 1.5 mg/kg, administered ≥2 d after first treatment dose (step-up dose 3) of 1.5 mg/kg, between Days 7-15.
In subsequent treatment cycles following Cycle 1, the subcutaneous Tec treatment doses of 3.0 mg/kg are administered as follows:
   From Cycle 2 (C2), Q4W dosing (Day 1 only) for all participants (regardless of clinical response).

When talquetamab SC and teclistamab SC are to be administered on the same day, the two drugs are to be given at least 15 minutes apart (it is suggested to administer talquetamab first).

### Arm C (PVd) Dosing Schedule

The medications will be administered in 28-day treatment cycles:
Elotuzumab IV: 10 mg/kg QW for Cycles 1 and 2, and 20 mg/kg Q4W for Cycles 3+
Pomalidomide PO: 4 mg/day on Days 1-21 of each cycle. There must be ≥7 d between the course of pomalidomide for each cycle.
Dexamethasone PO: For ≤75 years of age (between 3-24 h before elotuzumab), 28 mg QW for C1-C2: for C3+, 28 mg on D1, 40 mg on D5, D15, D22
Dexamethasone PO: For >75 years of age (between 3-24 h before Elotuzumab), 8 mg QW for C1-C2; for for C3+, 8 mg on D1, 20 mg on D5, D15, D22

When elotuzumab, pomalidomide, and dexamethasone are administered on the same day: dexamethasone will be administered 3 to 24 hours prior to elotuzumab; pomalidomide will be self-administered by the participant at approximately the same time of day.

### Efficacy Evaluations

Efficacy assessments will occur per IMWG criteria (2016) as defined in the protocol using data from serum, urine, bone marrow, and imaging (if applicable). Responses or progression will be evaluated by investigators, use of a computerized algorithm, and by an IRC; assessment by the IRC will be used as the primary analysis.

### Primary Outcome Measures:

Progression Free Survival (PFS) [Time Frame: Up to 7 years 2 months]
   PFS is defined as the duration from the date of randomization to either progressive disease or death, whichever comes first.

### Secondary Outcome Measures:

Overall Response Rate (ORR) [Time Frame: Up to 7 years 2 months]
   ORR is defined as the percentage of participants with best overall response of partial response (PR) or better according to international myeloma working group (IMWG) response criteria.
Complete Response (CR) or Better Rate [Time Frame: Up to 7 years 2 months]
   CR or better is defined as the percentage of participants with best overall response of CR or better according to IMWG response criteria.
Very Good Partial Response (VGPR) or Better Rate [Time Frame: Up to 7 years 2 months]
   VGPR or better is defined as the percentage of participants with best overall response of VGPR or better rate according to IMWG response criteria.
Minimal Residual Disease (MRD)-negative CR Rate [Time Frame: Up to 7 years 2 months]
   MRD-negative CR is defined as the percentage of participants who achieve both CR or better and MRD negativity at a threshold of 10\hat{ }−5 at any timepoint after the date of randomization and before disease progression or start of subsequent antimyeloma therapy (SST).
Overall Survival (OS) [Time Frame: Up to 7 years 2 months]
   OS is defined as the time from randomization to the date of participant's death.
Progression Free Survival on Next-line Therapy (PFS2) [Time Frame: Up to 7 years 2 months]
   PFS2 is defined as time from randomization to progression on the next line of therapy or death, whichever comes first.
Time to Next Treatment (TTNT) [Time Frame: Up to 7 years 2 months]
   TTNT is defined as the time from randomization to the start of SST.
Serum Concentration of Talquetamab and Teclistamab [Time Frame: Up to 7 years 2 months]
   Serum concentration of talquetamab and teclistamab will be reported.
Number of Participants with Anti-drug Antibodies (ADAs) to Talquetamab and Teclistamab [Time Frame: Up to 7 years 2 months]
   Number of participants with ADAs to talquetamab and teclistamab will be reported.
Time to Sustained Worsening in Symptoms, Functioning, and Health-related Quality of Life (HRQoL) as Assessed by Multiple Myeloma Symptom and Impact Questionnaire (MySIm-Q) [Time Frame: Up to 7 years 2 months]
   Time to sustained worsening in symptoms, functioning and HRQoL is defined as the interval from the date of randomization to the start date of meaningful change. The MySIm-Q is a disease-specific patient-reported outcome (PRO) assessment complementary to the European Organization for Research and Treatment of Cancer Quality of Life Questionnaire Core-30 item (EORTC-QLQ-C30). It includes 17 items resulting in a symptom subscale and an impact subscale.
Time to Sustained Worsening in Symptoms, Functioning, and Health-related Quality of Life (HRQoL) as Assessed by EORTC-QLQ-C30 [Time Frame: Up to 7 years 2 months]
   Time to sustained worsening in symptoms, functioning and HRQoL is defined as the interval from the date of randomization to the start date of meaningful change. EORTC-QLQ-C30 Version 3 includes 30 items that make up 5 functional scales (physical, role, emotional, cognitive, and social), 1 global health status scale, 3 symptom scales (pain, fatigue, and nausea or vomiting), and 5 single symptom items (dyspnea, insomnia, appetite loss, constipation, and diarrhea) and a single impact item (financial difficulties). The recall period is 7 days (“past week”), and responses are reported using a verbal and numeric rating scales. The item and scale scores are transformed to a 0 to 100 scale. A high scale score represents a higher response level.
Time to Sustained Worsening in Symptoms, Functioning, and Health-related Quality of Life (HRQoL) as Assessed by EuroQol Five Dimension Questionnaire 5-Level (EQ-5D-5L) [Time Frame: Up to 7 years 2 months]
   Time to sustained worsening in symptoms, functioning and HRQoL is defined as the interval from the date of randomization to the start date of meaningful change. The EQ-5D-5L is a 5-item questionnaire that assesses 5 domains including mobility, self-care, usual activities, pain or discomfort, and anxiety or depression plus a visual analog scale rating “health today” with anchors ranging from 0 (worst imaginable health state) to 100 (best imaginable health state).
Time to Sustained Worsening in Symptoms, Functioning, and Health-related Quality of Life (HRQoL) as Assessed by Patient Global Impression—Severity (PGI-S) [Time Frame: Up to 7 years 2 months]
   Time to sustained worsening in symptoms, functioning and HRQoL is defined as the interval from the date of randomization to the start date of meaningful change. The PGI-S will be used as an anchor, external criterion, to determine meaningful change in scores for the MySIm-Q and EORTC-QLQ-C30 in this population. The response options are presented as a 5-point verbal rating scale from “none” to “very severe.”
Time to Sustained Worsening in Symptoms, Functioning, and Health-related Quality of Life (HRQoL) as Assessed by Epstein Taste Survey [Time Frame: Up to 7 years 2 months]
   Time to sustained worsening in symptoms, functioning and HRQoL is defined as the interval from the date of randomization to the start date of meaningful change. The epstein taste survey consists of 17 items from the full 71 item PRO instrument, specific to taste changes. developed for use in patients with head and neck cancer as a composite of the Vanderbilt Head and Neck Symptom Survey.
Change from Baseline in Symptoms, Functioning, and Health-related Quality of Life (HRQoL) as Assessed by MySIm-Q [Time Frame: Up to 7 years 2 months]
   Change from baseline in symptoms, functioning, and HRQoL as assessed by MySIm-Q will be reported. The MySIm-Q is a disease-specific patient-reported outcome (PRO) assessment complementary to the European Organization for Research and Treatment of Cancer Quality of Life Questionnaire Core-30 item (EORTC-QLQ-C30). It includes 17 items resulting in a symptom subscale and an impact subscale.
Change from Baseline in Symptoms, Functioning, and Health-related Quality of Life (HRQoL) as Assessed by EORTC-QLQ-C30 [Time Frame: Up to 7 years 2 months]
   Change from baseline in symptoms, functioning, and HRQoL as assessed by EORTC-QLQ-C30 will be reported. The EORTC-QLQ-C30 Version 3 includes 30 items that make up 5 functional scales (physical, role, emotional, cognitive, and social), 1 global health status scale, 3 symptom scales (pain, fatigue, and nausea or vomiting), and 5 single symptom items (dyspnea, insomnia, appetite loss, constipation, and diarrhea) and a single impact item (financial difficulties). The recall period is 7 days (“past week”), and responses are reported using a verbal and numeric rating scales. The item and scale scores are transformed to a 0 to 100 scale. A high scale score represents a higher response level. Thus, a high score for a functional scale represents a high or healthy level of functioning and a high score for the global health status represents high HRQoL, but a high score for a symptom scale or item represents a high level of symptomatology or problems.
Change from Baseline in Symptoms, Functioning, and Health-related Quality of Life (HRQoL) as Assessed by EQ-5D-5L [Time Frame: Up to 7 years 2 months]
   Change from baseline in symptoms, functioning, and HRQoL as assessed by EQ-5D-5L will be reported. The EQ-5D-5L is a 5-item questionnaire that assesses 5 domains including mobility, self-care, usual activities, pain or discomfort, and anxiety or depression plus a visual analog scale rating “health today” with anchors ranging from 0 (worst imaginable health state) to 100 (best imaginable health state).
Change from Baseline in Symptoms, Functioning, and Health-related Quality of Life (HRQoL) as Assessed by PGI-S [Time Frame: Up to 7 years 2 months]
   Change from baseline in symptoms, functioning, and HRQoL as assessed by PGI-S will be reported. The PGI-S will be used as an anchor, external criterion, to determine meaningful change in scores for the MySIm-Q and EORTC-QLQ-C30 in this population. The response options are presented as a 5-point verbal rating scale from “none” to “very severe.”
Change from Baseline in Symptoms, Functioning, and Health-related Quality of Life (HRQoL) as Assessed by Epstein Taste Survey [Time Frame: Up to 7 years 2 months]
   Change from baseline in symptoms, functioning, and HRQoL as assessed by epstein taste survey will be reported. The epstein taste survey consists of 17 items from the full 71 item PRO instrument, specific to taste changes. developed for use in patients with head and neck cancer as a composite of the Vanderbilt Head and Neck Symptom Survey.
Percentage of Participants With Meaningful Improvement in HRQoL as Assessed by EORTC-QLQ-C30 [Time Frame: Up to 7 years 2 months]
   Percentage of participants with meaningful improvement in HRQol as assessed by EORTC-QLQ-C30 will be reported. The EORTC-QLQ-C30 Version 3 includes 30 items that make up 5 functional scales (physical, role, emotional, cognitive, and social), 1 global health status scale, 3 symptom scales (pain, fatigue, and nausea or vomiting), and 5 single symptom items (dyspnea, insomnia, appetite loss, constipation, and diarrhea) and a single impact item (financial difficulties). The recall period is 7 days (“past week”), and responses are reported using a verbal and numeric rating scales. The item and scale scores are transformed to a 0 to 100 scale. A high scale score represents a higher response level. Thus, a high score for a functional scale represents a high or healthy level of functioning and a high score for the global health status represents high HRQoL, but a high score for a symptom scale or item represents a high level of symptomatology or problems.

| Arms | Assigned Interventions |
|---|---|
| Experimental: Arm A: | Drug: Talquetamab |
| Talquetamab + Pomalidomide | Talquetamab will be |
| (Tal-P) | administered as a SC injection. |
| Participants will receive talquetamab | Other Name: JNJ-64407564 |
| as subcutaneous (SC) injections; | Other Name: Talvey |
| pomalidomide will be self- | Drug: Pomalidomide |
| administered as a single dose orally; | Pomalidomide will be |
| dexamethasone may be given orally | administered orally. |
| or intravenously as a pretreatment | Other Name: Pomalyst |
| medication and study drug. | Other Name: Imnovid |
| | Drug: Dexamethasone |
| | Dexamethasone will be |
| | administered either orally or |
| | intravenously. |
| Experimental: Arm B: | Drug: Talquetamab |
| Talquetamab + Teclistamab | Talquetamab will be |
| (Tal-Tec) | administered as a SC injection. |
| Participants will receive teclistamab | Other Name: JNJ-64407564 |
| in combination with talquetamab | Other Name: Talvey |
| both as SC injection; | Drug: Teclistamab |
| dexamethasone may be given | Teclistamab will be |
| orally or intravenously as a | administered as a SC injection. |
| pretreatment medication and study | Other Name: JNJ-64007957 |
| drug. | Other Name: Tecvayli |
| | Drug: Dexamethasone |
| | Dexamethasone will be |
| | administered either orally or |
| | intravenously. |
| Active Comparator: Arm C: | Drug: Pomalidomide |
| Elotuzumab + Pomalidomide + | Pomalidomide will be |
| Dexamethasone (EPd) or | administered orally. |
| Pomalidomide + Bortezomib + | Other Name: Pomalyst |
| Dexamethasone (PVd) | Other Name: Imnovid |
| Participants will either receive | Drug: Elotuzumab |
| elotuzumab intravenous (IV) | Elotuzumab will be |
| injection in combination with | administered intravenously. |
| pomalidomide and dexamethasone | Other Name: Empliciti |
| orally; or pomalidamide orally in | Drug: Dexamethasone |
| combination with bortezomib SC | Dexamethasone will be |
| injection and dexamethasone orally | administered either orally or |
| as per investigator choice. | intravenously. |
| Dexamethasone will be | Drug: Bortezomib |
| administered as a pretreatment | Bortezomib will be |
| | Other Name: Velcade |

### Inclusion Criteria:

Documented multiple myeloma as defined by the criteria below: (a) multiple myeloma diagnosis according to the international myeloma working group (IMWG) diagnostic criteria (b) measurable disease at screening as assessed by central laboratory, defined by any of the following; (i) serum M-protein level greater than or equal to (>=) 0.5 gram per deciliter (g/dL); or (ii) urine M-protein level >=200 milligram (mg) per 24 hours; or (iii) light chain multiple myeloma without measurable M-protein in the serum or the urine: serum immunoglobulin (Ig) free light chain (FLC) >=10 milligrams per deciliter (mg/dL) and abnormal serum Ig kappa lambda FLC ratio
Relapsed or refractory disease as defined below: a) Relapsed disease is defined as an initial response to prior treatment, followed by confirmed progressive disease (PD) by IMWG criteria greater than (>) 60 days after cessation of treatment. b) Refractory disease is defined as less than (<) 25 percent (%) reduction in M-protein or confirmed PD by IMWG criteria during previous treatment or less than or equal to (<=) 60 days after cessation of treatment
Documented evidence of PD or failure to achieve a minimal response to the last line of therapy based on investigator's determination of response by IMWG criteria on or after their last regimen
Have an Eastern Cooperative Oncology Group (ECOG) performance status score of 0, 1, or 2 at screening and immediately prior to the start of administration of study treatment
A participant must agree not to be pregnant, breastfeeding, or planning to become pregnant while enrolled in this study or within 6 months after the last dose of study treatment

### Exclusion Criteria:

Contraindications or life-threatening allergies, hypersensitivity, or intolerance to any study drug or its excipients
Stroke, transient ischemic attack, or seizure within 6 months prior to signing informed consent form (ICF)
Major surgery or had significant traumatic injury within 2 weeks prior to the start of administration of study treatment, or will not have fully recovered from surgery, or has major surgery planned during the time the participant is expected to be treated in the study or within 2 weeks after administration of the last dose of study treatment
A maximum cumulative dose of corticosteroids of >=140 mg of prednisone or equivalent within 14-day period before the first dose of study drug
Known active central nervous system (CNS) involvement or exhibits clinical signs of meningeal involvement of multiple myeloma. If either is suspected, negative whole brain magnetic resonance imaging (MRI) and lumbar cytology are required

1. A method of treating multiple myeloma in a subject in need thereof, comprising administering to the subject a combination dosing regimen comprising a therapeutically effective amount of a BCMA×CD3 bispecific antibody and a therapeutically effective amount of a GPRC5D×CD3 bispecific antibody.

2. The method of claim 1, wherein the BCMA×CD3 bispecific antibody comprises a BCMA binding domain comprising the HCDR1 of SEQ ID NO: 4, the HCDR2 of SEQ ID NO: 5, the HCDR3 of SEQ ID NO: 6, the LCDR1 of SEQ ID NO: 7, the LCDR2 of SEQ ID NO: 8 and the LCDR3 of SEQ ID NO: 9, and a CD3 binding domain comprising the HCDR1 of SEQ ID NO: 14, the HCDR2 of SEQ ID NO: 15, the HCDR3 of SEQ ID NO: 16, the LCDR1 of SEQ ID NO: 17, the LCDR2 of SEQ ID NO: 18 and the LCDR3 of SEQ ID NO: 19.

3. The method of claim 1, wherein the BCMA×CD3 bispecific antibody comprises a BCMA binding domain comprising a heavy chain variable region (VH) having the amino acid sequence of SEQ ID NO: 10 and a light chain variable region (VL) having the amino acid sequence of SEQ ID NO: 11, and the CD3 binding domain comprises a heavy chain variable region (VH) having the amino acid sequence of SEQ ID NO: 20 and a light chain variable region (VL) having the amino acid sequence of SEQ ID NO: 21.

4. The method of claim 1, wherein the BCMA×CD3 bispecific antibody is an IgG1, an IgG2, an IgG3 or an IgG4 isotype.

5. The method of claim 1, wherein the BCMA×CD3 bispecific antibody is an IgG4 isotype.

6. The method of claim 1, wherein the BCMA×CD3 bispecific antibody comprises one or more substitutions in its Fc region.

7. The method of claim 1, wherein the BCMA×CD3 bispecific antibody is an IgG4 isotype and comprises S228P, F234A and L235A substitutions in its Fc region.

8. The method of claim 1, wherein the BCMA×CD3 bispecific antibody is an IgG4 isotype and comprises S228P, F234A, L235A, F405L and R409K substitutions in its Fc region.

9. The method of claim 1, wherein the Fc region of the BCMA-specific IgG4 antibody from which the BCMA-binding arm is derived comprises S228P, L234A and L235A substitutions in its Fc region.

10. The method of claim 1, wherein the Fc region of the CD3-specific IgG4 antibody from which the CD3-binding arm is derived comprises S228P, L234A, L235A, F405L, and R409K substitutions in its Fc region.

11. The method of claim 1, wherein the BCMA×CD3 bispecific antibody comprises a first heavy chain (HC1) having the amino acid sequence of SEQ ID NO: 12, a first light chain (LC1) having the amino acid sequence of SEQ ID NO: 13, a second heavy chain (HC2) having the amino acid sequence of SEQ ID NO: 22 and a second light chain (LC2) having the amino acid sequence of SEQ ID NO: 23.

12. The method of claim 1, wherein the BCMA×CD3 bispecific antibody comprises a first heavy chain (HC1) having at least 90% identity to the amino acid sequence of SEQ ID NO: 12, a first light chain (LC1) having at least 90% identity to the amino acid sequence of SEQ ID NO: 13, a second heavy chain (HC2) having at least 90% identity to the amino acid sequence of SEQ ID NO: 22 and a second light chain (LC2) having at least 90% identity to the amino acid sequence of SEQ ID NO: 23.

13. The method of claim 1, wherein the BCMA×CD3 bispecific antibody comprises a first heavy chain (HC1) having at least 95% identity to the amino acid sequence of SEQ ID NO: 12, a first light chain (LC1) having at least 95% identity to the amino acid sequence of SEQ ID NO: 13, a second heavy chain (HC2) having at least 95% identity to the amino acid sequence of SEQ ID NO: 22 and a second light chain (LC2) having at least 95% identity to the amino acid sequence of SEQ ID NO: 23.

14. The method of claim 1, wherein the BCMA×CD3 bispecific antibody comprises a first heavy chain (HC1) having at least 98% identity to the amino acid sequence of SEQ ID NO: 12, a first light chain (LC1) having at least 98% identity to the amino acid sequence of SEQ ID NO: 13, a second heavy chain (HC2) having at least 98% identity to the amino acid sequence of SEQ ID NO: 22 and a second light chain (LC2) having at least 98% identity to the amino acid sequence of SEQ ID NO: 23.

15. The method of claim 1, wherein the BCMA×CD3 bispecific antibody is teclistamab.

16. The method of claim 1, wherein the GPRC5D×CD3 bispecific antibody comprises a GPRC5D binding domain comprising the HCDR1 of SEQ ID NO: 24, the HCDR2 of SEQ ID NO: 25, the HCDR3 of SEQ ID NO: 26, the LCDR1 of SEQ ID NO: 27, the LCDR2 of SEQ ID NO: 28 and the LCDR3 of SEQ ID NO: 29, and a CD3 binding domain comprising the HCDR1 of SEQ ID NO: 14, the HCDR2 of SEQ ID NO: 15, the HCDR3 of SEQ ID NO: 16, the LCDR1 of SEQ ID NO: 17, the LCDR2 of SEQ ID NO: 18 and the LCDR3 of SEQ ID NO: 19.

17. The method of claim 1, wherein the GPRC5D×CD3 bispecific antibody comprises a GPRC5D binding domain comprising a heavy chain variable region (VH) having the amino acid sequence of SEQ ID NO: 30 and a light chain variable region (VL) having the amino acid sequence of SEQ ID NO: 31, and the CD3 binding domain comprises a heavy chain variable region (VH) having the amino acid sequence of SEQ ID NO: 20 and a light chain variable region (VL) having the amino acid sequence of SEQ ID NO: 21.

18. The method of claim 1, wherein the GPRC5D×CD3 bispecific antibody is an IgG1, an IgG2, an IgG3 or an IgG4 isotype.

19. The method of claim 1, wherein the GPRC5D×CD3 bispecific antibody is an IgG4 isotype.

20. The method of claim 1, wherein the GPRC5D×CD3 bispecific antibody comprises one or more substitutions in its Fc region.

21. The method of claim 1, wherein the GPRC5D×CD3 bispecific antibody is an IgG4 isotype and comprises S228P, F234A and L235A substitutions in its Fc region.

22. The method of claim 1, wherein the GPRC5D×CD3 bispecific antibody is an IgG4 isotype and comprises S228P, F234A, L235A, F405L and R409K substitutions in its Fc region.

23. The method of claim 1, wherein the Fc region of the GPRC5D-specific IgG4 antibody from which the GPRC5D-binding arm is derived comprises S228P, L234A and L235A substitutions in its Fc region.

24. The method of claim 1, wherein the Fc region of the CD3-specific IgG4 antibody from which the CD3-binding arm is derived comprises S228P, L234A, L235A, F405L, and R409K substitutions in its Fc region.

25. The method of claim 1, wherein the GPRC5D×CD3 bispecific antibody comprises a first heavy chain (HC1) having the amino acid sequence of SEQ ID NO: 32, a first light chain (LC1) having the amino acid sequence of SEQ ID NO: 33, a second heavy chain (HC2) having the amino acid sequence of SEQ ID NO: 22 and a second light chain (LC2) having the amino acid sequence of SEQ ID NO: 23.

26. The method of claim 1, wherein the GPRC5D×CD3 bispecific antibody comprises a first heavy chain (HC1) having at least 90% identity to the amino acid sequence of SEQ ID NO: 32, a first light chain (LC1) having at least 90% identity to the amino acid sequence of SEQ ID NO: 33, a second heavy chain (HC2) having at least 90% identity to the amino acid sequence of SEQ ID NO: 22 and a second light chain (LC2) having at least 90% identity to the amino acid sequence of SEQ ID NO: 23.

27. The method of claim 1, wherein the GPRC5D×CD3 bispecific antibody comprises a first heavy chain (HC1) having at least 95% identity to the amino acid sequence of SEQ ID NO: 32, a first light chain (LC1) having at least 95% identity to the amino acid sequence of SEQ ID NO: 33, a second heavy chain (HC2) having at least 95% identity to the amino acid sequence of SEQ ID NO: 22 and a second light chain (LC2) having at least 95% identity to the amino acid sequence of SEQ ID NO: 23.

28. The method of claim 1, wherein the GPRC5D×CD3 bispecific antibody comprises a first heavy chain (HC1) having at least 98% identity to the amino acid sequence of SEQ ID NO: 32, a first light chain (LC1) having at least 98% identity to the amino acid sequence of SEQ ID NO: 33, a second heavy chain (HC2) having at least 98% identity to the amino acid sequence of SEQ ID NO: 22 and a second light chain (LC2) having at least 98% identity to the amino acid sequence of SEQ ID NO: 23.

29. The method of claim 1, wherein the GPRC5D×CD3 bispecific antibody is talquetamab.

30. The method of claim 1, wherein the subject has relapsed or refractory multiple myeloma.

31. The method of claim 1, wherein the subject has received at least three prior lines of therapy.

32. The method of claim 1, wherein the subject has received at least four prior lines of therapy.

33. The method of claim 1, wherein the subject has received at least five prior lines of therapy (penta-drug exposed).

34. The method of claim 1, wherein the subject has received at least three prior lines of therapy, including a proteasome inhibitor, an immunomodulatory agent and an anti-CD38 monoclonal antibody.

35. The method of claim 1, wherein the subject has received at least four prior lines of therapy, including a proteasome inhibitor, an immunomodulatory agent and an anti-CD38 monoclonal antibody.

36. The method of claim 1, wherein the subject has extramedullary disease (EMD).

37. The method of claim 1 comprising subcutaneously administering to the subject one or more step-up doses of the BCMA×CD3 bispecific antibody prior to administering a treatment dose of the BCMA×CD3 bispecific antibody.

38. The method of claim 1 comprising subcutaneously administering to the subject a treatment dose of the BCMA×CD3 bispecific antibody weekly (QW).

39. The method of claim 1 comprising subcutaneously administering to the subject a treatment dose of the BCMA×CD3 bispecific antibody every two weeks (Q2W).

40. The method of claim 1 comprising subcutaneously administering the BCMA×CD3 bispecific antibody weekly (QW) at a treatment dose of from about 750 μg/kg to about 1500 μg/kg.

41. The method of claim 1 comprising subcutaneously administering the BCMA×CD3 bispecific antibody weekly (QW) at a treatment dose of about 750 μg/kg.

42. The method of claim 1 comprising subcutaneously administering the BCMA×CD3 bispecific antibody weekly (QW) at a treatment dose of about 1500 μg/kg.

43. The method of claim 1 comprising subcutaneously administering the BCMA×CD3 bispecific antibody every two weeks (Q2W) at a treatment dose of from about 1500 μg/kg to about 3000 μg/kg.

44. The method of claim 1 comprising subcutaneously administering the BCMA×CD3 bispecific antibody every two weeks (Q2W) at a treatment dose of about 1500 μg/kg.

45. The method of claim 1 comprising subcutaneously administering the BCMA×CD3 bispecific antibody every two weeks (Q2W) at a treatment dose of about 3000 μg/kg.

46. The method of claim 37 comprising subcutaneously administering 2 or 3 step-up doses of the BCMA×CD3 bispecific antibody prior to subcutaneously administering the treatment dose.

47. The method of claim 37 comprising subcutaneously administering step-up doses of 60 μg/kg and 300 μg/kg of the BCMA×CD3 bispecific antibody prior to subcutaneously administering a treatment dose.

48. The method of claim 37 comprising subcutaneously administering step-up doses of 60 μg/kg and 300 μg/kg and 1500 μg/kg of the BCMA×CD3 bispecific antibody prior to subcutaneously administering a treatment dose.

49. The method of claim 37 comprising subcutaneously administering step-up doses of the BCMA×CD3 bispecific antibody 2-4 days apart from each other.

50. The method of claim 1 comprising subcutaneously administering to the subject one or more step-up doses of the GPRC5D×CD3 bispecific antibody prior to administering a treatment dose of the GPRC5D×CD3 bispecific antibody.

51. The method of claim 1 comprising subcutaneously administering to the subject a treatment dose of the GPRC5D×CD3 bispecific antibody weekly (QW).

52. The method of claim 1 comprising subcutaneously administering to the subject a treatment dose of the GPRC5D×CD3 bispecific antibody every two weeks (Q2W).

53. The method of claim 1 comprising subcutaneously administering the GPRC5D×CD3 bispecific antibody weekly (QW) at a treatment dose of from about 200 μg/kg to about 400 μg/kg.

54. The method of claim 1 comprising subcutaneously administering the GPRC5D×CD3 bispecific antibody weekly (QW) at a treatment dose of about 200 μg/kg.

55. The method of claim 1 comprising subcutaneously administering the GPRC5D×CD3 bispecific antibody weekly (QW) at a treatment dose of about 400 μg/kg.

56. The method of claim 1 comprising subcutaneously administering the GPRC5D×CD3 bispecific antibody every two weeks (Q2W) at a treatment dose of about 800 μg/kg.

57. The method of claim 50 comprising subcutaneously administering 2 or 3 step-up doses of the GPRC5D×CD3 bispecific antibody prior to subcutaneously administering the treatment dose.

58. The method of claim 50 comprising subcutaneously administering step-up doses of 10 μg/kg and 60 μg/kg of the GPRC5D×CD3 bispecific antibody prior to subcutaneously administering a treatment dose.

59. The method of claim 50 comprising subcutaneously administering step-up doses of 10 μg/kg and 60 μg/kg and 400 μg/kg of the GPRC5D×CD3 bispecific antibody prior to subcutaneously administering a treatment dose.

60. The method of claim 50 comprising subcutaneously administering step-up doses of the GPRC5D×CD3 bispecific antibody 2-4 days apart from each other.

61. The method of claim 1 comprising subcutaneously administering the BCMA×CD3 bispecific antibody every two weeks (Q2W) at a treatment dose of 3000 μg/kg and subcutaneously administering the GPRC5D×CD3 bispecific antibody every two weeks (Q2W) at a treatment dose of 800 μg/kg.

62. The method of claim 1 comprising subcutaneously administering the BCMA×CD3 bispecific antibody every two weeks (Q2W) at a treatment dose of 1500 μg/kg and subcutaneously administering the GPRC5D×CD3 bispecific antibody every two weeks (Q2W) at a treatment dose of 800 μg/kg.

63. The method of claim 1 comprising subcutaneously administering the BCMA×CD3 bispecific antibody weekly (QW) at a treatment dose of 1500 μg/kg and subcutaneously administering the GPRC5D×CD3 bispecific antibody weekly (QW) at a treatment dose of 800 μg/kg.

64. The method of claim 1 comprising subcutaneously administering the BCMA×CD3 bispecific antibody weekly (QW) at a treatment dose of 1500 μg/kg and subcutaneously administering the GPRC5D×CD3 bispecific antibody weekly (QW) at a treatment dose of 400 μg/kg.

65. The method of claim 1 comprising subcutaneously administering the BCMA×CD3 bispecific antibody weekly (QW) at a treatment dose of 1500 μg/kg and subcutaneously administering the GPRC5D×CD3 bispecific antibody weekly (QW) at a treatment dose of 200 μg/kg.

66. The method of claim 1 comprising subcutaneously administering the BCMA×CD3 bispecific antibody weekly (QW) at a treatment dose of 750 μg/kg and subcutaneously administering the GPRC5D×CD3 bispecific antibody weekly (QW) at a treatment dose of 200 μg/kg.

67. The method of claim 61 comprising subcutaneously administering one or more step-up doses of the BCMA×CD3 bispecific antibody prior to subcutaneously administering a treatment dose; and subcutaneously administering one or more step-up doses of the GPRC5D×CD3 bispecific antibody prior to subcutaneously administering a treatment dose.

68. The method of claim 61 comprising subcutaneously administering 2 or 3 step-up doses of the BCMA×CD3 bispecific antibody prior to subcutaneously administering a treatment dose; and subcutaneously administering 2 or 3 step-up doses of the GPRC5D×CD3 bispecific antibody prior to subcutaneously administering a treatment dose.

69. The method of claim 61 comprising subcutaneously administering step-up doses of 60 μg/kg and 300 μg/kg of the BCMA×CD3 bispecific antibody prior to subcutaneously administering a treatment dose; and subcutaneously administering step-up doses of 10 μg/kg and 60 μg/kg of the GPRC5D×CD3 bispecific antibody prior to subcutaneously administering a treatment dose.

70. The method of claim 61 comprising subcutaneously administering step-up doses of 60 μg/kg and 300 μg/kg and 1500 μg/kg of the BCMA×CD3 bispecific antibody prior to subcutaneously administering a treatment dose; and subcutaneously administering step-up doses of 10 μg/kg and 60 μg/kg and 300 μg/kg of the GPRC5D×CD3 bispecific antibody prior to subcutaneously administering a treatment dose.

71. The method of claim 61 comprising subcutaneously administering step-up doses of 60 μg/kg and 300 μg/kg and 1500 μg/kg of the BCMA×CD3 bispecific antibody prior to subcutaneously administering a treatment dose; and subcutaneously administering step-up doses of 10 μg/kg and 60 μg/kg and 400 μg/kg of the GPRC5D×CD3 bispecific antibody prior to subcutaneously administering a treatment dose.

72. The method of claim 67, wherein the step-up doses of the BCMA×CD3 bispecific antibody are administered 2-4 days apart from each other, and the step-up doses of the GPRC5D×CD3 bispecific antibody are administered 2-4 days apart from each other.

73. The method of claim 1, wherein each treatment dose of the BCMA×CD3 bispecific antibody is administered on the same day as each treatment dose of the GPRC5D×CD3 bispecific antibody.

74. The method of claim 1, wherein each step-up dose of the BCMA×CD3 bispecific antibody is administered on the same day as each step-up dose of the GPRC5D×CD3 bispecific antibody.

75. The method of claim 1, wherein the first treatment dose of the BCMA×CD3 bispecific antibody and the first treatment dose of the GPRC5D×CD3 bispecific antibody are administered subcutaneously on Cycle 1 Day 1 of a 28-day cycle, wherein:
subsequent treatment doses of the GPRC5D×CD3 bispecific antibody are administered Q2W at a dose amount of 800 μg/kg subcutaneously, e.g., on Days 1 and 15 (±3 days) of each 28-day cycle, and
subsequent treatment doses of the BCMA×CD3 bispecific antibody are administered Q2W at a dose amount of 3000 μg/kg subcutaneously, e.g., on Days 1 and 15 (±3 days) of a 28-day cycle.

76. The method of claim 75 comprising subcutaneously administering step-up doses of 60 μg/kg and 300 μg/kg and 1500 μg/kg of the BCMA×CD3 bispecific antibody prior to subcutaneously administering said first treatment dose; and subcutaneously administering step-up doses of 10 μg/kg and 60 μg/kg and 400 μg/kg of the GPRC5D×CD3 bispecific antibody prior to subcutaneously administering said first treatment dose, wherein said first treatment doses are administered 2 to 4 days after administration of the last step-up dose of each bispecific antibody.

77. The method of claim 1, wherein the method achieves a partial response, a very good partial response, a complete response or a stringent complete response in the subject, as determined by IMWG response criteria.

78. The method of claim 77, wherein the subject has extramedullary disease (EMD).

79. The method of claim 1, wherein the method achieves an overall response rate of at least 70% in a population of subjects with relapsed or refractory multiple myeloma (RRMM).

80. The method of claim 79, wherein the subjects have extramedullary disease (EMD).

81. The method of claim 1, wherein the method achieves an overall response rate of at least 80% in a population of subjects with RRMM.

82. The method of claim 81, wherein the subjects have extramedullary disease (EMD).

83. The method of claim 1, wherein the method achieves an overall response rate of at least 85% in a population of subjects with RRMM.

84. The method of claim 83, wherein the subjects have extramedullary disease (EMD).

85. The method of claim 1, wherein the method achieves an overall response rate of at least 90% in a population of subjects with RRMM.

86. The method of claim 1, wherein the method achieves an overall response rate of at least 95% in a population of subjects with RRMM.

87. The method of claim 1, wherein the method achieves a complete response or a stringent complete response rate of at least 20% in a population of subjects with RRMM.

88. The method of claim 87, wherein the subjects have extramedullary disease (EMD).

89. The method of claim 1, wherein the method achieves a complete response or a stringent complete response rate of at least 25% in a population of subjects with RRMM.

90. The method of claim 89, wherein the subjects have extramedullary disease (EMD).

91. The method of claim 1, wherein the method achieves a complete response or a stringent complete response rate of at least 30% in a population of subjects with RRMM.

92. The method of claim 91, wherein the subjects have extramedullary disease (EMD).
